SEQUENCE LISTING (28 sequences)

SEQ ID NO: 1 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 2 - protein, 5 aa, synthetic construct
  REGION 1..5: note = VH CDR1
DYYMK

SEQ ID NO: 3 - protein, 17 aa, synthetic construct
  REGION 1..17: note = MIRG 1231 - VH CDR2
DIIPSNGATFYNQKFKG

SEQ ID NO: 4 - protein, 11 aa, synthetic construct
  REGION 1..11: note = MIRG 1231 - VH CDR3
SHLLRASWFAY

SEQ ID NO: 5 - protein, 17 aa, synthetic construct
  REGION 1..17: note = MIRG 1231 - VL CDR1
ESSQSLLNSGNQKNYLT

SEQ ID NO: 6 - protein, 16 aa, synthetic construct
  REGION 1..16: note = CD123- VL CDR1
KSSQSLLNSGNQKNYL

SEQ ID NO: 7 - protein, 7 aa, synthetic construct
  REGION 1..7: note = MIRG 1231 - VH CDR2
WASTRES

SEQ ID NO: 8 - protein, 9 aa, synthetic construct
  REGION 1..9: note = MIRG 1231 - VH CDR3
QNDYSYPYT

SEQ ID NO: 9 - protein, 120 aa, synthetic construct [sequence omitted]

SEQ ID NO: 10 - protein, 113 aa, synthetic construct
  REGION 1..113: note = MIRG 1231-VL
DIVMTQSPDSLAVSLGERATINCKSSQSLLNSGNQKNYLTWYQQKPGQPPKPLIYWASTRESGVPDRFSGSGSGTDFTLTISSLQAEDVAVYYCQNDYSYPYTFGQGTKLEIK

SEQ ID NO: 11 - protein, 120 aa, synthetic construct
  REGION 1..120: note = Flotetuzumab-VH
EVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSS

SEQ ID NO: 12 - protein, 113 aa, synthetic construct
  REGION 1..113: note = FlotetuzumabVL
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIK

SEQ ID NO: 13 - protein, 120 aa, synthetic construct
  REGION 1..120: note = vibecotabam-VH
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSS

SEQ ID NO: 14 - protein, 113 aa, synthetic construct
  REGION 1..113: note = vibecotabam-VL
DFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIK

SEQ ID NO: 15 - protein, 493 aa, synthetic construct
  REGION 1..493: note = anti-CD123 CAR
MALPVTALLLPLALLLHAARPDIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKGGGGSGGGGSGGGGSEVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - DNA, 22 bp, synthetic construct
  misc_feature 1..22: note = gRNA E51K - 1 (m1guide1)
  source 1..22: mol_type = other DNA
tatagctcacacacaatttctg

SEQ ID NO: 17 - DNA, 21 bp, synthetic construct
  misc_feature 1..21: note = gRNA E51K - 2 (Cas12a gRNA)
  source 1..21: mol_type = other DNA
acactggctatagctcacaca

SEQ ID NO: 18 - DNA, 20 bp, synthetic construct
  misc_feature 1..20: note = gRNA S59P - 1 (m2guide1)
  source 1..20: mol_type = other DNA
tctgcggctgataagatacg

SEQ ID NO: 19 - DNA, 20 bp, synthetic construct
  misc_feature 1..20: note = gRNA S59P - 2 (m2guide2)
  source 1..20: mol_type = other DNA
tgataagatacggccattta

SEQ ID NO: 20 - DNA, 20 bp, synthetic construct
  misc_feature 1..20: note = grna s59p - 3 (m2guide3)
  source 1..20: mol_type = other DNA
catttagtatgagagataac

SEQ ID NO: 21 - DNA, 20 bp, synthetic construct
  misc_feature 1..20: note = gRNA R84E - 1 (guide 1)
  source 1..20: mol_type = other DNA
ctggttgatgtggcaggctc

SEQ ID NO: 22 - DNA, 20 bp, synthetic construct
  misc_feature 1..20: note = gRNA R84E - 2(guide 2)
  source 1..20: mol_type = other DNA
aggctcaccggttgggtggt

SEQ ID NO: 23 - DNA, 20 bp, synthetic construct
  misc_feature 1..20: note = gRNA R84E - 3(guide 3)
  source 1..20: mol_type = other DNA
tcaccggttgggtggtaaga

SEQ ID NO: 24 - DNA, 20 bp, synthetic construct
  misc_feature 1..20: note = gRNA TRAC
  source 1..20: mol_type = other DNA
agagtctctcagctggtaca

SEQ ID NO: 25 - protein, 45 aa, synthetic construct
  REGION 1..45: note = CD8-Hinge
TTTPAPRPPTPAPTIASQPLSLRPEACRPAAGGAVHTRGLDFACD

SEQ ID NO: 26 - protein, 21 aa, synthetic construct
  REGION 1..21: note = CD8-TM
IYIWAPLAGTCGVLLLSLVIT

SEQ ID NO: 27 - protein, 42 aa, synthetic construct
  REGION 1..42: note = 4-1BB cytoplasmic domain
KRGRKKLLYIFKQPFMRPVQTTQEEDGCSCRFPEEEEGGCEL

SEQ ID NO: 28 - protein, 113 aa, synthetic construct
  REGION 1..113: note = Cd3-zeta
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0326818 A1
(43) Pub. Date: Oct. 23, 2025

(54) DISCERNIBLE CELL SURFACE PROTEIN VARIANTS FOR USE IN CELL THERAPY

(71) Applicants: Universität Basel, BALE (CH); CIMEIO THERAPEUTICS AG, BASEL (CH)

(72) Inventors: Rosalba LEPORE, BASEL (CH); Lukas JEKER, RIEHEN (CH); Stefanie URLINGER, MUNCHEN (DE); Emmanuelle LANDMANN, BINNINGEN (CH); Alessandro SINOPOLI, SAINT-LOUIS (FR); Amélie WIEDERKEHR, RANSPACH LE BAS (FR); Anna DEVAUX, SAINT-LOUIS (FR); Anna CAMUS, BASEL (CH); Anna HAYDN, ZURICH (CH); Romina MATTER-MARONE, RIEHEN (CH)

(21) Appl. No.: 18/681,419

(22) PCT Filed: Aug. 5, 2022
(86) PCT No.: PCT/EP2022/072168

§ 371 Date: Feb. 5, 2024

(30) Foreign Application Priority Data
Aug. 6, 2021 (EP) 21306100.5
Feb. 25, 2022 (EP) 22158991.4

(51) Int. Cl. C07K 14/705; A61K 35/17; A61K 40/11; A61K 40/31; A61K 40/42; C12N 5/071; C12N 5/0783

(52) U.S. Cl. CPC C07K 14/70596; A61K 35/17; A61K 40/11; A61K 40/31; A61K 40/4224; C12N 5/0636; C12N 5/0686; A61K 2239/15; C12N 2510/00

(57) ABSTRACT

The present invention relates to the use of cells having discernible surface protein with engineered or naturally occurring mutation(s) but functional surface protein for use in therapy. The present invention also relates to the use of cells having discernible CD123 surface protein variants but functional surface protein for use in therapy, in particular adoptive cell therapy.

### REFERENCE TO AN ELECTRONIC SEQUENCE LISTING

The present application contains a Sequence Listing that has been submitted electronically and is hereby incorporated by reference in its entirety. The electronic Sequence Listing is named Sequence_Listing_ST26, was created on Sep. 9, 2024, and is 37,386 bytes in size.

### FIELD OF THE INVENTION

The present invention relates to the use of cells having discernible surface protein with engineered or naturally occurring mutation(s) but functional surface protein for use in therapy. The present invention also relates to the use of cells having discernible CD123 surface protein variants but functional surface protein for use in therapy, in particular adoptive cell therapy.

### STATEMENT REGARDING FUNDING

The project leading to this application has received funding from the European Research Council (ERC) under the European Union's Horizon 2020 research and innovation programme (grant agreement No 818806).

### BACKGROUND OF THE INVENTION

Cellular therapy is emerging as the third pillar of medicine after small molecule therapy and treatments based on biologics such as recombinant proteins including antibodies. Cellular therapy can be used in oncology for treating hematopoietic malignant diseases, but also other applications such as the treatment of genetic diseases, solid organ tumors and autoimmune diseases are under development. However, cellular therapy can be associated with severe unwanted side effects.

Indeed, while cancer immunotherapy with chimeric antigen receptor (CAR) T cells has been successful in targeting and eradicating malignant cells expressing a specific antigen, it does often not discriminate between normal and malignant cells and thus induces destruction of the normal hematopoietic system. Targeted therapies, which include antibody-based therapies, such as conventional monoclonal antibodies, multispecific antibodies, such as T cell engagers (e.g. BiTE's) and cellular therapies, such as CAR cells (e.g. CAR T-cells, CAR NK cells or CAR macrophages), eliminate all cells expressing the target molecule. However, most cancer cell surface antigens are shared with normal hematopoietic or other cells. Thus, to identify targets to kill diseased cells including tumors while avoiding damage to healthy cells is a major challenge for targeted therapies. In particular, in myeloid diseases including myeloid malignancies such as myelodysplastic syndrome (MDS), acute myeloid leukemia (AML) or Blastic Plasmacytoid Dendritic Cell Neoplasm (BPDCN) cell surface antigens such as CD33, or CD123 are shared with normal myeloid progenitors. Therefore, immunotherapy targeting CD33 or CD123 antigen for MDS, AML or PBDCN can be associated with depletion of normal hematopoietic cells in addition to malignant cells in patients (Gill S. I. Best practice & Research Clinical Hematology, 2019). As a consequence, targeted immunotherapy including mAbs, T cell engagers or CAR T have mostly been elusive, in part owing to the absence of truly disease-specific surface antigens (Gill S. I. Best practice & Research Clinical Hematology, 2019).

To regenerate normal hematopoiesis depleted through CD33-CAR T cell transfer, CD33 CAR T cell resistant hematopoietic cells are being engineered in such a way that the entire CD33 gene is knocked out (Kim et al. 2018. Cell. 173:1439-53). However, CD33 has a constitutive inhibitory effect on myeloid cells through its immunoreceptor tyrosine-based inhibitory motif (ITIM) signaling domain. Thus, it remains unclear how well the loss of CD33 may be tolerated. CD33-knock-out (CD33 KO) engineered cells transplanted in patients could present long-term functional defects and very heterogeneous outcome (WO2018/160768, Kim et al. 2018. Cell. 173:1439-53, Borot et al. 2019. PNAS. 116:11978-87, Humbert et al. 2019. Leukemia. 33:762-808). In fact, the frequency of CD33 KO cells decreased in the two monkeys for which a long-term observation was reported. This could indicate functional impairment of CD33 KO cells, for instance through reduced engraftment of CD33 KO long-term repopulating HSC (LT-HSC) or through a competitive disadvantage (Kim et al. 2018. Cell. 173:1439-53). In addition, the number of cell surface antigens with dispensable function is very limited and loss of said redundant cell surface antigen can induce antigen negative relapse. CD19-negative relapses are observed in approximately 30% of patients receiving CD19-targeted CAR T therapy (Orlando et al. 2018 Nat Med 24: 1504-6). Dual targeting of CD19 and CD123 can prevent antigen-loss relapses (Ruella et al. 2016 J Clin Invest 126:3814-26).

WO2018/160768 describes an approach in which hematopoietic cells are engineered in a manner in that entire epitopes on the surface antigen CD33 are deleted. Antigens with such larger deletions can be expected not to have equivalent function as the respective wild-type surface antigen. WO2014/138805 discloses certain variants of CD123 which show reduced binding to certain antibodies. Cell Reports (2014) 8: 410⁻⁹ discloses certain amino acids involved in binding of CSL362 to CD123. US20190185573 discloses the antibody binding properties of certain variants of CD123. None of these documents discloses the use of such variants as contemplated in the present disclosure. EP3769816 discloses antibody chains with homology to the CDRs of certain molecules disclosed in the present disclosure. Blood (2017) 130 Suppl1: 2625 discloses a method of treating cancer utilizing anti-CD123 CAR-T cells. Such treatment does not make use of any variants of CD123.

The inventors in previous patents applications showed that a single amino acid difference in surface protein variants can be engineered into hematopoietic cells to change the antigenicity and be discriminated by specific and selective antibodies (WO2017/186718, WO2018/083071). Contrary to CD33 KO cells, the surface protein variants in these cells retain their normal expression and function and enable to target surface proteins with important non-redundant functions.

### SUMMARY OF THE INVENTION

One of the objectives of the present disclosure is to develop a safer method for the treatment of malignancies, in particular cancer, hematological malignancies, myeloid diseases. The inventors thus sought variations of surface protein which are immunologically distinguishable while retaining normal function and where amino acid changes originate due to single or multiple nucleotide variations. In particular, the inventors identified rationally designed and naturally occurring variants of CD123 and showed that these mutations change the antigenicity of CD123 to a specific antibody while retaining its normal expression and function, in particular, binding of Interleukin-3 (IL-3).

The present invention relates to a mammalian cell or a population of cells expressing a first isoform of a surface protein, preferably CD123 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein and preferably wherein said first and second isoform are functional.

In a particular embodiment, the present invention relates to the mammalian cell or population of cells, preferably hematopoietic stem cells for use in a medical treatment in a patient in need thereof wherein said medical treatment comprises: administering a therapeutically efficient amount of said cell or population of cells expressing said first isoform to said patient in need thereof, in combination with a therapeutically efficient amount of a depleting agent comprising at least a first antigen-binding region that binds specifically to said second isoform to specifically deplete patient cells expressing second isoform, preferably to restore normal hematopoiesis after immunotherapy in the treatment of hematopoietic disease, preferably malignant hematopoietic disease such as acute myeloid leukemia (AML), blastic plasmacytoid dendritic cell neoplasm (BPDCN), or B-acute lymphoblastic leukemia (B-ALL).

In another particular embodiment, the present invention relates to the mammalian cell or population of cells for use in a medical treatment in a patient in need thereof wherein said medical treatment comprises: administering a therapeutically efficient amount of said cell or population of cells expressing said first isoform to said patient in need thereof in combination with a therapeutically efficient amount of a depleting agent comprising at least a second antigen-binding region that binds specifically to said first isoform to specifically deplete transferred cells expressing first isoform, preferably for use in adoptive cell transfer therapy, more preferably for the treatment of malignant hematopoietic disease such as acute myeloid leukemia (AML), blastic plasmacytoid dendritic cell neoplasm (BPDCN), or B-acute lymphoblastic leukemia (B-ALL), again more preferably wherein said depleting agent is administered subsequently to said cell or population of cells expressing said first isoform of surface protein to avoid eventual severe side effects such as graft-versus-host disease due to the transplantation.

In another aspect, the present invention relates to a pharmaceutical composition comprising a mammalian cell, preferably a hematopoietic stem cell or an immune cell such as T-cell as described above and preferably a depleting agent and a pharmaceutically acceptable carrier.

The present invention also relates to a depleting agent for use in preventing or reducing the risk of severe side effects in a patient having received a cell expressing a first isoform of a surface protein, wherein said patient's native cells express a second isoform of surface protein, and wherein said depleting agent comprises at least a second antigen-binding region which binds specifically to said first isoform and does not bind to said second isoform, preferably wherein said surface protein is CD123.

In another aspect, the present invention relates to a depleting agent for use in selectively depleting the host cells in a patient in need thereof wherein said patient's native cells express a second isoform of a surface protein and wherein said depleting agent comprises at least a first antigen-binding region which binds specifically to said second isoform, preferably wherein said surface protein is CD123 and wherein said first antigen-binding region of said depleting agent binds specifically to an epitope including the amino acids T48, D49, E51, A56, D57, Y58, 559, M60, P61, A62, V63, N64, T82, R84, V85, A86, N87, P89, F90, S91 of SEQ ID NO: 1, more preferably wherein said first antigen-binding region comprises:
a) an antibody heavy chain variable domain (VH) comprising the three CDRs VHCDR1, VHCDR2 and VHCDR3 wherein VHCD1 is SEQ ID NO: 2, VHCD2 is SEQ ID NO: 3 and VHCDR3 is SEQ ID NO: 4; and
b) an antibody light chain variable domain (VL) comprising the three CDRs VLCDR1, VLCDR2 and VLCDR3 wherein VLCDR1 is SEQ ID NO: 5 or 6, VLCDR2 is SEQ ID NO: 7, VLCDR3 is SEQ ID NO: 8, again more preferably wherein said first antigen binding region comprises a heavy chain variable domain comprising or consisting of any one of amino acid sequences selected from SEQ ID NO: 9, 11 and 13 and/or a light chain variable domain comprising or consisting of any one of amino acid sequences selected from SEQ ID NO: 10, 12 and 14.

### FIGURE LEGENDS

FIG. 1: A) CD123 per-residue relative solvent accessibility (RSA). RSA was computed for the CSL362-bound and CSL362-free state based on the X-ray structure of the CD123-CSL362 complex (PDB ID: 4JZJ). Data are shown for the N-terminal domain. Arrows indicate the RSA of residues E51, S59 and R84. B) 3D structure of the CD123-CSL362 complex and selected amino acid variants. CD123 residues E51, S59 and R84 are shown as sticks. The structure of CSL362 antibody is shown as molecular surface.

FIG. 2: Flow cytometry of the CD123 variants and controls (HEK, HEK-CD123) stained with two monoclonal antibody clones MIRG123 and 6H6. The variants are coded according to their abolished (underlined), weak (circle), or strong (Asterix) binding to MIRG123. Controls HEK and HEK-CD123 in grey.

FIG. 3: Quantification of the FACS plots in FIG. 2. The % MIRG123⁺ 6H6⁺ double positive (upper right quadrant) cells are plotted for controls and all variants. 6H6 binding was unaltered (see FIG. 2). Summary of 3 independent experiments. Mutation variants are qualified as non-binders (underlined, <1% MIRG123⁺ 6H6⁺), weak binder (circle, 1-20% MIRG123⁺ 6H6) and strong binder (Asterix, >20% MIRG123⁺ 6H6).

FIG. 4: CD123 isoform variants prevent FcgRIIIA activation. Relative luminescence signal measured after cocultivation of HEK, HEK-CD123 (wildtype) and HEK CD123 variant isoforms with Jurkat/FcγRIIIa/NFAT-Luc reporter cells and MIRG123. RLU is normalized to the signal measured with HEK-CD123 (wildtype). Mutation variants are qualified as non-binders (underlined, <1% MIRG123⁺ 6H6⁺), weak binder (circle, 1-20% MIRG123⁺ 6H6) and strong binder (Asterix, >20% MIRG123⁺ 6H6).

FIG. 5: CD123 isoform variants prevent CD3/CSL362 BiTE-mediated T-cell activation. Stable target cell lines HEK, HEK-CD123 and CD123 variants were co-cultured with human primary pan T cells at an E:T ratio of 10:1 in the presence of 300 ng/mL CD3/CSL362 BiTE. Shown is a summary of % CD69⁺ T cells measured after co-culture with HEK, HEK-CD123 or all CD123 variants. The data are normalized to % CD69⁺ cells in the presence of HEK target cells. Error bars show mean±SD. Data represent 5 independent blood donors and experiments with 2 technical replicates per group.

FIG. 6: CD123 isoform variants are protected from CD3/CSL362 BiTE-mediated human T cell killing. Stable target cell lines HEK, HEK-CD123 and CD123 variants were co-cultured with human primary pan T cells at an E:T ratio of 10:1 in the presence of 300 ng/mL CD3/CSL362 BiTE. Shown is the specific BiTE-mediated killing of HEK, HEK-CD123 and all CD123 variants in % after 72 h co-culture. Error bars show mean±SD. Data represent 5 independent blood donors and experiments with 2 technical replicates per group. CD123 variant expression was confirmed by FACS with a monoclonal antibody 6H6.

FIG. 7: Non-viral HDR-mediated integration of the CD123-specific second-generation CAR into Exon 1 of the TRAC locus using CRISPR/Cas9 and design of the CAR construct based on CSL362.

FIG. 8: Result of the FACS analysis representing effector CAR T cell activation (CD69) after one day co-culture of effector CAR T cells with different target cells. Summary of % CD69⁺ CAR T cells either alone (effector T cells) or in the presence of HEK, HEK-CD123 or all CD123 variants after 24 h co-culture. The data are normalized to % CD69⁺ cells in the presence of HEK target cells. Control T cells not expressing the CAR were not activated.

FIG. 9: Quantification underlying data of FIG. 8. Quantification of specific killing measured by flow cytometry of HEK, HEK-CD123 and its variants by CD123-specific CAR T cells at day one of co-culture. Error bars show mean±SD. Data from 3 independent blood donors and experiments with 2 technical replicates per group.

FIG. 10: A) BLI sensograms of CD123 WT and E51T to CSL362. B) Binding levels of CD123_WT and variants at 50 nM top concentration to captured CSL362 antibody (captured at the same level) at 280 seconds. C) BLI sensograms of CD123_WT and E51T to control antibody 6H6. D) Binding levels of CD123_WT and variants to captured 6H6 antibody (normalized to captured/loading level) at 250 seconds. E) BLI sensograms of E51Q and E51T to CSL362. F) Binding levels of CD123_variants at higher concentration than B (300 nM as top concentration) to captured CSL362 antibody (captured at the same level) at 280 seconds. G) Binding levels of CD123_WT at 50 nM top concentration and CD123_variants at 300 nM top concentration to captured Flotetuzumab (captured at the same level) at 250 seconds.

FIG. 11: A) BLI sensograms of CD123_WT and E51T binding to IL3. B) Binding levels of IL3 to captured biotinylated CD123 variants at 250 seconds (normalized to captured/loading level).

FIG. 12: Thermal unfolding is monitored using SYPRO Orange. In the inset first derivative data. Fluorescence monitored in PBS buffer 5× Sypro orange dye. Protein concentration is 0.25 mg/mL. The heating rate used was 1.5° C./min ramp, recording from 25 to 95° C. A) Thermal unfolding of CD123_WT and CD123 variants (E51T, E51K, E51Q, S59P, S59E, and R84E). B) Thermal unfolding of CD123 variants (E51A, S59R, S59F, S59Y, R84Q, and R84T).

FIG. 13: Bulk TF-1 cells were cultured as control (no crRNA), as KO (crRNA but no HDRT) or KI (crRNA plus KI HDRT, E51K or E51T). Control cells cultured with IL-3 or GM-CSF remained MIRG123+, 6H6+. KO cells cultured with GM-CSF largely remained MIRG123−, 6H6+. In contrast, in cultures containing KO cells cultured with IL-3 gradually, a MIRG123+, 6H6+ cell population became detectable. On day 6 the MIRG123+, 6H6+ population dominated, demonstrating that cells expressing the CD123 receptor had a competitive advantage in presence of IL3. In KI cells (E51K or E51T) the population of MIRG123− 6H6+ but also a population of MIRG123+ 6H6+ cells gradually increased with IL3. This was much less pronounced in cells cultured with GM-CSF. Thus, KI cells (MIRG123− 6H6+) have a functional receptor.

FIG. 14: TF-1 cell (wild type, knock-out, as well as E51K and a E51T knock-in cells) were tested for their capacity of being stimulated with IL3. TF-1 knock-in cells expressing the E51K and E51T variants of CD123 can proliferate upon add of hIL3 to a similar degree as TF-1 cells expressing wild-type CD123. Knock-out cells only show a minimal response to hIL3.

FIG. 15: Antibody MIRG123 was tested for its capability to bind to TF-1 cell (wild type, knock-out, as well as E51K and a E51T knock-in cells). MIRG123 leads to a dose-dependent proliferation blocking and apoptosis of IL3-stimulated wild type TF-1 cells. In contrast, TF-1 knock-in cells expressing the E51K and E51T variants of CD123 are efficiently protected from the blocking effects of MIRG123.

FIG. 16: HSPCs cells with a E51K and a E51T knock-in could be successfully generated. Plotted are the % of knock-in cells 2 and 5 days after electroporation (EP).

FIG. 17: HSCs with a E51K and a E51T knock-in show a loss of binding to antibody MIRG123 but preserved CD123 expression as assessed by the control antibody 6H6.

FIG. 18: Also LT-HSCs (long term repopulating hematopoietic stem cells), as well as MPP1 cells (CD34⁺ CD38−CD90− CD45RA−) and MPP2 cells (CD34⁺CD38− CD90− CD45RA+) are successfully edited.

FIGS. 19 and 20: Co-culture of human effector T cells with control or edited HSPC cells (E:T=3:1) in the presence of a CD3/CSL362 BiTE leads to a reduction of wild-type HSPCs upon treatment with the BiTE, as measured by quantification of the flow cytometry plots. In contrast HSCs expressing the CD123 E51K or E51T variants are protected and enriched. Error bars show mean±SD. Data were generated with 2 independent donors.

### DETAILED DESCRIPTION OF THE INVENTION

Immunotherapy is a promising therapy to treat cancer, genetic and autoimmune diseases. Immunodepleting agent such as engineered immune cells directed to tumor antigen are administered into a patient to target and kill tumor cells. However, as tumor surface proteins are also expressed at the surface of normal cells including hematopoietic cells, this strategy can induce severe side effects to the patients, e.g. by altering hematopoiesis. To restore hematopoiesis in the patient, hematopoietic cells can be subsequently transplanted into the patient. However, the binding of the depleting agent not only to the diseased cells but also to the newly transplanted healthy cells can limit the maximal tolerated dose or limit the use to treatment before transplantation of healthy cells. Alternatively, transplanted cells need to be resistant to said immunodepleting agent in order not to be targeted and eliminated by it. Thus, the inventor selected cells resistant to said immunodepleting agent used in immunotherapy while retaining their function to restore normal hematopoiesis in the patient.

The inventors develop a method to identify functional allelic variants in the genetic sequence encoding the surface protein region responsible for the binding of a specific depleting agent. Such variants can be naturally occurring polymorphisms and/or designed and engineered variants. Different isoforms of surface proteins can be selected or generated. Said first isoform of a surface protein encoded by a nucleic acid with said polymorphism is not recognized by a specific depleting agent. This variant allele particularly does not alter the function of the surface protein. Thus, said depleting agent can be used to bind specifically to the one isoform and not the other isoform thereby depleting specifically cells expressing one isoform. For example, if the depleting agent binds specifically to the second isoform, but not the first isoform, said depleting agent will specifically deplete cells expressing said second isoform. In another embodiment, said first isoform can be recognized by a second agent and thus this second agent can be used to deplete specifically cells expressing the first isoform, but not second isoform. The cells expressing the first isoform of the surface protein encoded by at least one variant allele is advantageously used in medical treatment in a patient having cells expressing a second isoform, in particular for depleting specifically transplanted or patient cells by using a second or first agent respectively.

### Depleting Agent

The present disclosure relates to an agent comprising an antigen binding region which binds specifically to one isoform of a surface protein on a cell and does not bind to another isoform of said surface protein. Such agent is referred to herein as “depleting agent”. Both isoform of said surface protein are functional, i.e. the surface protein if functional with respect to at least one relevant property of said surface protein. Preferably both isoforms of said surface protein have that same function, i.e. they are functionally indistinguishable.

The two isoforms of the surface protein differ however with respect to binding to the depleting agent. The depleting agent only binds specifically to one of the isoforms of said surface protein. The isoforms can therefore be described as functional identical, but immunological distinguishable.

The first isoform and the second isoform of said surface protein may be polymorphic alleles of said surface protein. Preferably, the first isoform and the second isoform of said surface protein are naturally occurring polymorphic alleles of said surface protein. Also preferably, the first isoform and the second isoform of said surface protein are single nucleotide polymorphism (SNP) alleles.

The first isoform and the second isoform of said surface protein may also be genetically engineered alleles. Preferably the first isoform and the second isoform of said surface protein differ by one, two, three, four or five amino acids. Most preferably the first isoform and the second isoform of said surface protein differ by one amino acid.

Various methods can be used to determine the mutation that is to be introduced into the surface protein to generate the second isoform. For example, mutations can be randomly inserted into the surface protein, followed by the functional and immunological screening of the variants generated. Alternatively, mutations can be rationally designed, for example by analysis of the secondary or tertiary protein structure of the surface protein.

The depleting agent comprising an antigen binding region which binds specifically to one isoform of a surface protein on a cell and does not bind to another isoform of said surface protein. The depleting agent of the present disclosure can be divided into two main categories. First, the depleting agent can be a polypeptide comprising an antigen binding region. Said polypeptide may consist of one or more polypeptide chains. Preferably said polypeptide comprising an antigen binding region is an antibody. Said polypeptide comprising an antigen binding region may also be an antibody fragment, an antibody drug conjugate, or another variant of an antibody or scaffold. Exemplary antibody fragments and scaffolds include single domain antibodies, maxibodies, minibodies, intrabodies, diabodies, triabodies, tetrabodies, v-NAR, bis-scFv, camelid antibodies, ankyrins, centyrins, domain antibodies, lipocalins, small modular immuno-pharmaceuticals, maxybodies, Protein A and affilins.

Said polypeptide comprising an antigen binding region may also be a bispecific, biparatopic or multispecific antibody. Such molecules may also contain additional functional domains. For example said polypeptide comprising an antigen binding region may be a T cell engager, for example a BiTE. Said polypeptide comprising an antigen binding region may also be fused to a cytokine or a chemokine, or to the extracellular domain of a cell surface receptor.

Alternative, the depleting agent can be a cell comprising an antigen binding region. For example, the depleting agent can be a chimeric antigen receptor (CAR). In certain embodiments of the present disclosure said cell comprising an antigen binding region is a CAR T-cell, CAR NK cells or CAR macrophages. In a preferred embodiment of the present disclosure said cell comprising an antigen binding region is a CAR T-cell. In another preferred embodiment of the present disclosure said cell comprising an antigen binding region is a primary T cell comprising a CAR.

The depleting agent binds specifically to one isoform of the surface protein, but not the second isoform and thus specifically depletes cells expressing one isoform.

In certain embodiments, the present disclosure relates to an agent comprising a first antigen binding region which binds specifically to a second isoform of a surface protein and does not bind a first isoform. In other embodiments, the present disclosure also relates to an agent comprising a second antigen binding region which binds specifically to the first isoform and does not bind a second isoform.

The first and the second isoform of the surface protein may differ from each other by only one amino acid substitution. Said one amino acid difference between the first and the second isoform may also be the result of the presence of a single nucleotide polymorphism, such as a naturally occurring single nucleotide polymorphism. The first and the second isoform of the surface protein may also differ from each other by more than one amino acid, such as by two, by three or by more than three amino acids. The first and the second isoform of the surface protein may also differ from each other in that one of the isoforms has an insertion of one, of two, of three or of more than three amino acids compared to the other isoform. The first and the second isoform of the surface protein may also differ from each other in that one of the isoforms has a deletion of one, of two, of three or of more than three amino acids compared to the other isoform. In a preferred embodiment, said depleting agent is an antibody or an antigen-binding fragment.

The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies.

In natural antibodies of rodents and primates, two heavy chains are linked to each other by disulfide bonds, and each heavy chain is linked to a light chain by a disulfide bond. There are two types of light chains, lambda (k) and kappa (κ). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR).

The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate in the antibody binding site, or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

In specific embodiments, an antibody provided herein is an antibody fragment, and more particularly any protein including an antigen-binding domain of an antibody as disclosed herein. The antigen-binding domain may also be integrated into another protein scaffold Antibody fragments and scaffolds include, but are not limited to, Fv, Fab, F(ab′)2, Fab′, dsFv, scFv, sc(Fv)2, diabodies, single domain antibodies, maxibodies, minibodies, intrabodies, diabodies, triabodies, tetrabodies, v-NAR, bis-scFv, camelid antibodies, ankyrins, centyrins, domain antibodies, lipocalins, small modular immuno-pharmaceuticals, maxybodies, Protein A and affilins.

As used herein, an “antigen binding region” or “antigen-binding fragment of an antibody” means a part of an antibody, i.e. a molecule corresponding to a portion of the structure of the antibody, that exhibits antigen-binding capacity for a specific antigen, possibly in its native form; such fragment especially exhibits the same or substantially the same antigen-binding specificity for said antigen compared to the antigen-binding specificity of the corresponding four-chain antibody. The antigen-binding capacity can be determined by measuring the affinity between the antibody and the target fragment. This antigen-binding region may also be designated as “functional fragments” of antibodies.

The agents of the disclosure comprise antibodies and fragments thereof but also comprise artificial proteins with the capacity to bind antigens mimicking that of antibodies, also termed herein antigen-binding antibody mimetic. Antigen-binding antibody mimetics are organic compounds that specifically bind antigens, but that are not structurally related to antibodies. They are usually artificial peptides or small proteins with a molar mass of about 3 to 20 kDa.

The phrases “an antigen binding region recognizing an antigen” and “an antigen binding region having specificity for an antigen” are used interchangeably herein with the term “an antigen binding region which binds specifically to an antigen”. As used herein, the term “specificity” refers to the ability of an agent comprising an antigen binding region such as an antibody to detectably bind an epitope presented on an antigen.

“Appreciable affinity” or “specific binding” or “specifically bind to” includes binding with an affinity of about 10⁻⁸ M (KD) or stronger. Preferably, binding is considered specific when the binding affinity is between 10⁻⁸ M (KD) and 10⁻¹² M (KD), optionally between 10⁻⁸ M (KD) and 10⁻¹⁰ M (KD), in particular at least 10⁻⁸ M (KD). The affinity can be determined by various methods well known from the one skilled in the art. These methods include, but are not limited to, surface plasmon resonance (SPR), biolayer interferometry (BLI), microscale thermophoresis (MST) and Scatchard plot. Whether a binding domain specifically reacts with or binds to a target can be tested readily by, inter alia, comparing the reaction of said binding domain with a target protein or antigen with the reaction of said binding domain with proteins or antigens other than the target protein.

As used herein, the term “epitope” means the part of an antigen to which the antibody or antigen binding region thereof binds. The epitopes of protein antigens can be divided into two categories, conformational epitope and linear epitope. A conformational epitope corresponds to discontinuous sections of the antigen's amino acid sequence. A linear epitope corresponds to a continuous sequence of amino acids from the antigen.

In another aspect, it is further disclosed herein bispecific or multispecific molecules, such as bispecific antibodies or multispecific antibodies. For example, an antibody can be derivatized or linked to another functional molecule, e.g., another peptide or protein (e.g., another antibody or ligand for a receptor) to generate a bispecific molecule that binds to at least two different binding sites or target molecules. The antibody may in fact be derivatized or linked to more than one other functional molecule to generate multi-specific molecules that bind to more than two different binding sites and/or target molecules; such multi-specific molecules are also intended to be encompassed by the terms “bispecific molecule”, “bispecific antibody”, “biparatopic molecule”, “biparatopic antibody”, “multispecific molecule” and “multispecific antibody” as used herein. To create a bispecific molecule, an antibody of the invention can be functionally linked (e.g., by chemical coupling, genetic fusion, noncovalent association or otherwise) to one or more other binding molecules, such as another antibody, antibody fragment, peptide or binding mimetic, cytokine, chemokine or a receptor extracellular domain, such that a bispecific molecule results. Specific bispecific and multispecific molecules contemplated by the present disclosure are T cell engagers, such as bispecific T cell engager, for example a BiTE.

As used herein, an agent which does not bind to a particular isoform includes an agent which is not able to bind to cells expressing said particular isoform. In particular, said agent is labelled with a fluorescent marker or detected with a secondary antibody directed against said agent and the percentage of cells presenting said fluorescent marker or said secondary antibody staining at the surface detected by FACS analysis is determined as described in the experimental part. In order to monitor expression of the variant isoforms, cells were stained with two agents simultaneously, one binding the epitope where variants were introduced (e.g. anti-CD123 CSL362) and a second one that binds an epitope that is different from the one bound by the first agent (e.g. anti-CD123 clone 6H6). The second epitope remains unaltered and thus this staining serves as an expression control. As a non-binding control, cells were used that do not express the protein of interest (e.g. HEK cells). As a maximum binding control, cells that normally do not express the protein of interest were transfected with the wildtype isoform (e.g. HEK-CD123). Hence, in specific embodiments, said agent is not able to bind to cells expressing said particular isoform when the percentage of cells binding at their surface the agent (e.g. anti-CD123 antibody) coupled to fluorescent marker detecting by FACS analysis is below 10%, preferably below 5%, and more preferably below 1%, or below detectable limits. Binding is hereby measured by fluorescence in the FACS in the upper right quadrant (i.e. binding to both, the control agent and the agent of interest). The reduced binding also results in reduced fluorescence of said first but not said second agent.

In an alternative assay, the binding of two agents, one binding the epitope where variants were introduced (e.g. anti-CD123 antibody CSL362) and a second one that binds an epitope that is different from the one bound by the first agent (e.g. anti-CD123 clone 6H6) is measured label-free and in real-time on purified recombinant CD123 extracellular domains of the wildtype as well as the variant isoforms by bio-layer interferometry. Hence, in specific embodiments, said first agent is not able to bind the recombinant CD123 variant isoform extracellular domains when no relevant signal above background is detectable at antibody concentrations of 50 to 300 nM. Detectable binding of the second agent at 50 to 300 nM concentration to an invariant epitope serves as binding and integrity control.

Binding of said agent can result in depletion of the cell expressing the first isoform. Various mechanisms can lead to cell depletion. Antibody dependent cellular cytotoxicity (ADCC) results from binding of the agent to a target protein and activation of NK cells through the Fc part on the agent bound by an FcR expressed by NK cells. The Fc part of an immunoglobulin refers to the C-terminal region of an immunoglobulin heavy chain. The Fc part can be wildtype or engineered. Mutations of enhanced, engineered Fc parts are known in the art. For certain therapeutic situations, it is desirable to reduce or abolish the normal binding of the wild-type Fc region of an antibody, such as of a wild-type IgG Fc region to one or more or all of Fc receptors and/or binding to a complement component, such as C1 q in order to reduce or abolish the ability of the antibody to induce effector function. For instance, it may be desirable to reduce or abolish the binding of the Fc region of an antibody to one or more or all of the Fcy receptors, such as: FcyRI, FcyRIIa, FcyRIIb, FcyRIIIa. Effector function can include, but is not limited to, one or more of the following: complement dependent cytotoxicity (CDC), antibody-dependent cell-mediated cytotoxicity (ADCC), antibody-dependent cellular phagocytosis (ADCP), cytokine secretion, immune complex-mediated antigen uptake by antigen-presenting cells, binding to NK cells, binding to macrophages, binding to monocytes, binding to polymorphonuclear cells, direct signaling inducing apoptosis, crosslinking of target-bound antibodies, dendritic cell maturation, or T cell priming.

A reduced or abolished binding of an Fc region to an Fc receptor and/or to C1 q is typically achieved by mutating a wild-type Fc region, such as of an lgG1 Fc region, more particular a human lgG1 Fc region, resulting in a variant or engineered Fc region of said wild-type Fc region, e.g. a variant human lgG1 Fc region. Substitutions that result in reduced binding can be useful. For reducing or abolishing the binding properties of an Fc region to an Fc receptor, non-conservative amino acid substitutions, i.e. replacing one amino acid with another amino acid having different structural and/or chemical properties, are preferred.

Surrogate ADCC assays constitute an industry standard to quantitate an agent's potency to mediate ADCC as described in the experimental part. Engineered Jurkat reporter cells carry an NFAT-responsive luciferase gene and an Fc receptor. Binding of the Fc receptor to bound antibody results in NFAT induction and therefore a luciferase signal. Absence of binding does not result in a luciferase signal. Cells expressing either no target protein (e.g. HEK), the wildtype protein (e.g. HEK-CD123) or individual variants (e.g. CD123 variants) were incubated with the test agent (e.g. CSL362 or MIRG123) and mixed with the ADCC reporter cells. Then luciferase was measured to quantify the ADCC signal. The luciferase luminescence signals were normalized to the maximal signal observed in HEK-CD123. ADCC was measured with an ADCC Reporter Assay (Promega, Cat.No. G7015).

An alternative way of depleting target cells is through the use of T cell engager molecules. The inventors constructed a bispecific T cell engager using a CD123 binding site derived from CSL362 and a CD3 (OKT3) binding site as described in Hutmacher, Leuk Res, 2019. The same target cells used for the ADCC assay were used. Primary human T cells and the bispecific T cell engager were added. Activation of human T cells was quantified by FACS by determining the frequency of CD69 upregulation. In addition, specific killing was calculated as described in the methods.

The depleting agent according to the present disclosure binds specifically to one isoform of a surface protein and allows the depletion of cells expressing said isoform.

More preferably, in specific embodiments, said depleting agent according to the present disclosure does not bind to a first isoform of a cell surface protein but binds specifically to a second isoform of said cell surface protein and allows the depletion of said cells expressing said second isoform, in particular in methods of use as disclosed herein. In particular, said depleting agent which does not bind to a first isoform of a cell surface protein but binds specifically to a second isoform expressed in patient's cell is used to deplete patient's cells but not hematopoietic stem cells or their progeny expressing said first isoform transplanted to restore hematopoiesis in said patient.

In another specific embodiments, said depleting agent according to the present disclosure does not bind to a second isoform of a cell surface protein but binds specifically to a first isoform of said cell surface protein and allows the depletion of cells expressing said first isoform, in particular in methods of use as disclosed herein. In particular, said depleting agent which does not bind to a second isoform of a cell surface protein but binds specifically to a first isoform expressed in transplanted cells is used to deplete specifically transplanted cells to avoid eventual severe side effects such as graft-versus-host disease due to transplantation.

Selective depletion of cells expressing specific isoform of surface protein can be achieved without limitation by complement-dependent cytotoxicity (CDC), antibody-dependent cellular cytotoxicity (ADCC) or antibody-dependent cellular phagocytosis (ADCP).

In certain embodiments, the antigen binding region is coupled to an effector compound such as a drug or a toxin. Such conjugates are referred to herein as “immunoconjugates” or “antibody-drug conjugates” (ADC). A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, t. colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, maytansinoids, calicheamicins, indolinobenzodiazepines, pyrolobenzodiazepines, alpha-amanitin, microcystins, auristatins and puromycin and analogs or homologs thereof.

In another particular embodiment, said depleting agent is an immune cell harboring an antigen receptor such as a chimeric antigen receptor (CAR). Said immune cell may express a recombinant antigen binding region, also named antigen receptor on its cell surface. By “recombinant” is meant an antigen binding region which is not encoded by the cell in its native state, i.e. it is heterologous, non-endogenous. Expression of the recombinant antigen binding region can thus be seen to introduce a new antigen specificity to the immune cell, causing the cell to recognise and bind a previously unrecognized antigen. The antigen receptor may be isolated from any useful source. In certain embodiments of the present disclosure said cell comprising an antigen binding region is a CAR T-cell, a CAR NK cell or a CAR macrophage. In a preferred embodiment of the present disclosure said cell comprising an antigen binding region is a CAR T-cell. In another preferred embodiment of the present disclosure said cell comprising an antigen binding region is a primary T cell comprising a CAR.

In a particular embodiment, said recombinant antigen receptor is a chimeric antigen receptor (CAR). CARs are fusion proteins comprising an antigen-binding region, typically derived from an antibody, linked to the signaling domain of the TCR complex. CARs can be used to direct immune cells such T-cells or NK cells against a target antigen if a suitable antigen-binding region is selected.

The antigen-binding region of a CAR is typically based on a scFv (single chain variable fragment) derived from an antibody. In addition to an N-terminal, extracellular antibody-binding region, CARs typically may comprise a hinge domain, which functions as a spacer to extend the antigen-binding region away from the plasma membrane of the immune effector cell on which it is expressed, a transmembrane (TM) domain, an intracellular signaling domain (e.g. the signaling domain from the zeta chain of the CD3 molecule (CD3ζ) of the TCR complex, or an equivalent) and optionally one or more co-stimulatory domains which may assist in signaling or functionality of the cell expressing the CAR. Signaling domains from co-stimulatory molecules including CD28, OX-40 (CD134), and 4-1BB (CD137) can be added alone (second generation) or in combination (third generation) to enhance survival and increase proliferation of CAR modified immune cells.

The skilled person is able to select an appropriate antigen binding region as described above with which to redirect an immune cell to be used according to the invention. In a particular embodiment, the immune cell for use in the method of the invention is a redirected T-cell, e.g. a redirected CD8⁺ T-cell or a redirected CD4⁺ T-cell, or a redirected NK cell.

The inventors generated a CD123-specific CAR with the single-chain variable fragment (scFv) of clone CSL362, the CD8alpha hinge and transmembrane domain (Gen CD8A ENSG00000153563), the intracellular signaling moieties 4-1BB (Gen TNFRSF9 ENSG00000049249) and CD3zeta (Gen CD247 ENSG00000198821). Specific killing can be measured by determining the number of alive cells by flow cytometry as described in the examples. Specific killing was calculated according to the indicated formula: (1—Number of alive target cells in co-culture with CAR T cells/Number alive target cells in co-culture with control cells)*100.

Methods by which immune cells can be genetically modified to express a recombinant antigen binding region are well known in the art. A nucleic acid molecule encoding the antigen receptor may be introduced into the cell in the form of e.g. a vector, or any other suitable nucleic acid construct. Vectors, and their required components, are well known in the art. Nucleic acid molecules encoding antigen binding region can be generated using any method known in the art, e.g. molecular cloning using PCR. Antigen binding region sequences can be modified using commonly used methods, such as site-directed mutagenesis.

### Anti-CD123 Agent

Anti-CD123 agents are known in the art. For example, talacotuzumab (CSL362) is a humanized anti-CD123 antibody of CSL Limited and flotetuzumab is a bispecific T-cell engager developed by Macrogenics (Uy et al. Blood 137: 751-62). An anti-CD123/anti CAR-T is developed by Chongqing Precision Biotech. MB-102, an antiCD123/antiCD28 costimulatory cellular agent is developed by Mustang Bio. IMG-532 is an anti-CD123−ADC developed by Immunogen. UCART-123 is an anti-CD123 CAR-T developed by Cellectis. Vibecotamab is an antiCD123/antiCD3 bispecific antibody developed by Xencor. Other anti-CD123 CAR-T's are under development as well, including those of GeMoaB Monoclonals, Novartis (JEZ-567), Hrain Biotechnology (HRAIN-004) and Hebei Senlang Biotechnology. Bispecific molecules in development include APVO-436 of Aptevo Therapeutics and JNJ-63709178 of Johnson & Johnson. All these molecules can principally be used in the methods and compositions of the present disclosure, provided they can discriminate two isoforms of a cell surface protein according to the present disclosure.

Talacotuzumab (CSL362), flotetuzumab and vibecotamab are derivatives of the same parental antibody, with their CDRs having 98-100% sequence identity. Knowing the epitope of CSL362 we can therefore assume that flotetuzumab and vibecotamab will bind to the identical epitope.

In a particular embodiment, when said surface protein is CD123, said depleting agent which binds to said second isoform and does not bind to said first isoform as described above binds specifically to an epitope including the amino acids T48, D49, E51, A56, D57, Y58, 559, M60, P61, A62, V63, N64, T82, R84, V85, A86, N87, P89, F90, S91 of SEQ ID NO: 1.

In a preferred embodiment, said anti-CD123 agent comprises an antigen binding region comprising:
a) an antibody heavy chain variable domain (VH) comprising the three CDRs VHCDR1, VHCDR2 and VHCDR3 wherein VHCDR1 is SEQ ID NO: 2 (DYYMK), VHCDR2 is SEQ ID NO: 3 (DIIPSNGATFYNQKFKG) and VHCDR3 is SEQ ID NO: 4 (SHLLRASWFAY); and
b) an antibody light chain variable domain (VL) comprising the three CDRs VLCDR1, VLCDR2 and VLCDR3 wherein VLCDR1 is SEQ ID NO: 5 (ESSQSLLNSGNQKNYLT) or SEQ ID NO: 6 (KSSQSLLNSGNQKNYL), VLCDR2 is SEQ ID NO: 7 (WASTRES), VLCDR3 is SEQ ID NO: 8 (QNDYSYPYT).

It is further contemplated that the antigen-binding region may be further screened or optimized for their binding properties as above defined. In particular, it is contemplated that said antigen binding region thereof may have 1, 2, 3, 4, 5, 6, or more alterations in the amino acid sequence of 1, 2, 3, 4, 5, or 6 CDRs of monoclonal antibodies provided herein, in particular SEQ ID NO: 3-8. It is contemplated that the amino acid in position 1, 2, 3, 4, 5, 6, 7, 8, 9, or 10 of CDR1, CDR2, CDR3, CDR4, CDR5, or CDR6 of the VJ or VDJ region of the light or heavy variable region of antigen binding region may have an insertion, deletion, or substitution with a conserved or non-conserved amino acid. Such amino acids that can either be substituted or constitute the substitution are disclosed above.

In some embodiments, the amino acid differences are conservative substitutions, i.e., substitutions of one amino acid with another having similar chemical or physical properties (size, charge or polarity), which substitution generally does not adversely affect the biochemical, biophysical and/or biological properties of the antibody. In particular, the substitution does not disrupt the interaction of the antibody with CD123 antigen. Said conservative substitution(s) are advantageously chosen within one of the following five groups: Group 1-small aliphatic, non-polar or slightly polar residues (A, S, T, P, G); Group 2-polar, negatively charged residues and their amides (D, N, E, Q); Group 3-polar, positively charged residues (H, R, K); Group 4-large aliphatic, nonpolar residues (M, L, I, V, C); and Group 5-large, aromatic residues (F, Y, W).

In another preferred embodiment, said anti-CD123 agent comprises an antigen binding region which binds to the same epitope as an antigen binding region comprising:
a) an antibody heavy chain variable domain (VH) comprising the three CDRs VHCDR1, VHCDR2 and VHCDR3 wherein VHCDR1 is SEQ ID NO: 2 (DYYMK), VHCDR2 is SEQ ID NO: 3 (DIIPSNGATFYNQKFKG) and VHCDR3 is SEQ ID NO: 4 (SHLLRASWFAY); and
b) an antibody light chain variable domain (VL) comprising the three CDRs VLCDR1, VLCDR2 and VLCDR3 wherein VLCDR1 is SEQ ID NO: 5 (ESSQSLLNSGNQKNYLT) or SEQ ID NO: 6 (KSSQSLLNSGNQKNYL), VLCDR2 is SEQ ID NO: 7 (WASTRES), VLCDR3 is SEQ ID NO: 8 (QNDYSYPYT).

In another preferred embodiment, said anti-CD123 agent comprises an antigen binding region which has the same epitope specificity as an antigen binding region comprising:
a) an antibody heavy chain variable domain (VH) comprising the three CDRs VHCDR1, VHCDR2 and VHCDR3 wherein VHCDR1 is SEQ ID NO: 2 (DYYMK), VHCDR2 is SEQ ID NO: 3 (DIIPSNGATFYNQKFKG) and VHCDR3 is SEQ ID NO: 4 (SHLLRASWFAY); and
b) an antibody light chain variable domain (VL) comprising the three CDRs VLCDR1, VLCDR2 and VLCDR3 wherein VLCDR1 is SEQ ID NO: (ESSQSLLNSGNQKNYLT) or SEQ ID NO: 6 (KSSQSLLNSGNQKNYL), VLCDR2 is SEQ ID NO: 7 (WASTRES), VLCDR3 is SEQ ID NO: 8 (QNDYSYPYT).

In another preferred embodiment, said anti-CD123 agent comprises an antigen binding region which is immunologically indistinguishable from an antigen binding region comprising:
a) an antibody heavy chain variable domain (VH) comprising the three CDRs VHCDR1, VHCDR2 and VHCDR3 wherein VHCDR1 is SEQ ID NO: 2 (DYYMK), VHCDR2 is SEQ ID NO: 3 (DIIPSNGATFYNQKFKG) and VHCDR3 is SEQ ID NO: 4 (SHLLRASWFAY); and
b) an antibody light chain variable domain (VL) comprising the three CDRs VLCDR1, VLCDR2 and VLCDR3 wherein VLCDR1 is SEQ ID NO: 5 (ESSQSLLNSGNQKNYLT) or SEQ ID NO: 6 (KSSQSLLNSGNQKNYL), VLCDR2 is SEQ ID NO: 7 (WASTRES), VLCDR3 is SEQ ID NO: 8 (QNDYSYPYT).

In a more particular embodiment, said first antigen-binding region comprises a heavy chain variable domain comprising or consisting of any one of amino acid sequences selected from SEQ ID NO: 9, 11 and 13 and/or a light chain variable domain comprising or consisting of any one of amino acid sequences selected from SEQ ID NO: 10, 12 and 14.

In another particular embodiment, said first antigen-binding region comprises a heavy chain variable domain which binds to the same epitope as an antigen binding region comprising or consisting of any one of amino acid sequences selected from SEQ ID NO: 9, 11 and 13 and/or a light chain variable domain comprising or consisting of any one of amino acid sequences selected from SEQ ID NO: 10, 12 and 14.

In another particular embodiment, said first antigen-binding region has the same epitope specificity as an antigen binding region comprising or consisting of any one of amino acid sequences selected from SEQ ID NO: 9, 11 and 13 and/or a light chain variable domain comprising or consisting of any one of amino acid sequences selected from SEQ ID NO: 10, 12 and 14.

In another particular embodiment, said first antigen-binding region is immunologically indistinguishable from an antigen binding region comprising or consisting of any one of amino acid sequences selected from SEQ ID NO: 9, 11 and 13 and/or a light chain variable domain comprising or consisting of any one of amino acid sequences selected from SEQ ID NO: 10, 12 and 14.

Said first antigen binding region thereof with amino acid sequences having at least 90%, for example, at least 95%, 96%, 97%, 98%, or 99% identity to any one of the above defined amino acid sequences are also part of the present disclosure, typically first antigen binding region have at least equal or higher binding activities than said first antigen binding region consisting of heavy chain consisting of any one of amino acid sequences selected from SEQ ID NO: 9, 11 and 13 and light chain consisting of any one of amino acid sequences selected from SEQ ID NO: 10, 12 and 14.

In a particular embodiment, said anti-CD123 agent can be a bispecific CD123 antibody, comprising at least one first binding specificity for CD123, for example, one antigen-binding region of anti-CD123 as described herein and a second binding specificity for a second target epitope or target antigen. In particular, said bispecific antibody is a bifunctional fusion anti-CD123 and anti-CD3 as described in Kuo S. R., et al. Protein Eng. Des. Sel. 2012; 25:561-569, 137; Hussaini M., Blood. 2013; 122:360.; Chicili G. R., Sci. Transl. Med. 2015; 7:289ra82 and Al-Hussaini M. Blood. 2016; 127:122-131).

According to the present disclosure, said anti-CD123 agent can be an immune cell harboring an antigen receptor targeting CD123, such as a CAR targeting CD123, said antigen receptor comprising an antigen binding region as described above.

In specific embodiments, said immune cell (e.g. T cell) harboring a CAR targeting CD123 recognizes a second isoform of CD123 as expressed in a patient in need thereof, and does not recognize a first isoform of CD123. In particular said immune cell may bind specifically to an epitope including the amino acids T48, D49, E51, A56, D57, Y58, 559, M60, P61, A62, V63, N64, T82, R84, V85, A86, N87, P89, F90, S91 of SEQ ID NO: 1.

In specific embodiments, said anti-CD123 agent can be an immune cell (e.g. T cell) harboring a CAR, said CAR comprising an antigen-binding region, e.g. scFv, comprising.
a) an antibody heavy chain variable domain (VH) comprising the three CDRs VHCDR1, VHCDR2 and VHCDR3 wherein VHCD1 is SEQ ID NO: 2, VHCD2 is SEQ ID NO: 3 and VHCDR3 is SEQ ID NO: 4; and
b) an antibody light chain variable domain (VL) comprising the three CDRs VLCDR1, VLCDR2 and VLCDR3 wherein VLCDR1 is SEQ ID NO: 5 or 6, VLCDR2 is SEQ ID NO: 7, VLCDR3 is SEQ ID NO: 8.

In another specific embodiments, said anti-CD123 agent can be an immune cell (e.g. T cell) harboring a CAR, said CAR comprising an antigen-binding region, e.g. scFv, comprising an antigen binding region which binds to the same epitope as an antigen binding region comprising
a) an antibody heavy chain variable domain (VH) comprising the three CDRs VHCDR1, VHCDR2 and VHCDR3 wherein VHCD1 is SEQ ID NO: 2, VHCD2 is SEQ ID NO: 3 and VHCDR3 is SEQ ID NO: 4; and
b) an antibody light chain variable domain (VL) comprising the three CDRs VLCDR1, VLCDR2 and VLCDR3 wherein VLCDR1 is SEQ ID NO: 5 or 6, VLCDR2 is SEQ ID NO: 7, VLCDR3 is SEQ ID NO: 8.

In another specific embodiments, said anti-CD123 agent can be an immune cell (e.g. T cell) harboring a CAR, said CAR comprising an antigen-binding region, e.g. scFv, which has the same epitope specificity as an antigen binding region comprising
a) an antibody heavy chain variable domain (VH) comprising the three CDRs VHCDR1, VHCDR2 and VHCDR3 wherein VHCD1 is SEQ ID NO: 2, VHCD2 is SEQ ID NO: 3 and VHCDR3 is SEQ ID NO: 4; and
b) an antibody light chain variable domain (VL) comprising the three CDRs VLCDR1, VLCDR2 and VLCDR3 wherein VLCDR1 is SEQ ID NO: 5 or 6, VLCDR2 is SEQ ID NO: 7, VLCDR3 is SEQ ID NO: 8.

In another specific embodiments, said anti-CD123 agent can be an immune cell (e.g. T cell) harboring a CAR, said CAR comprising an antigen-binding region, e.g. scFv, which is immunologically indistinguishable from an antigen binding region comprising
a) an antibody heavy chain variable domain (VH) comprising the three CDRs VHCDR1, VHCDR2 and VHCDR3 wherein VHCD1 is SEQ ID NO: 2, VHCD2 is SEQ ID NO: 3 and VHCDR3 is SEQ ID NO: 4; and
b) an antibody light chain variable domain (VL) comprising the three CDRs VLCDR1, VLCDR2 and VLCDR3 wherein VLCDR1 is SEQ ID NO: 5 or 6, VLCDR2 is SEQ ID NO: 7, VLCDR3 is SEQ ID NO: 8.

In a more particular embodiment, said anti-CD123 agent can be an immune cell (e.g. T cell) harboring a CAR comprising said first antigen-binding region e.g. scFv comprising a heavy chain variable domain comprising or consisting of any one of amino acid sequences selected from SEQ ID NO: 9, 11 and 13 and/or a light chain variable domain comprising or consisting of any one of amino acid sequences selected from SEQ ID NO: 10, 12 and 14.

In another particular embodiment, said anti-CD123 agent can be an immune cell (e.g. T cell) harboring a CAR comprising said first antigen-binding region e.g. scFv, which binds to the same epitope as an antigen binding region comprising a heavy chain variable domain comprising or consisting of any one of amino acid sequences selected from SEQ ID NO: 9, 11 and 13 and/or a light chain variable domain comprising or consisting of any one of amino acid sequences selected from SEQ ID NO: 10, 12 and 14.

In another particular embodiment, said anti-CD123 agent can be an immune cell (e.g. T cell) harboring a CAR comprising said first antigen-binding region e.g. scFv, which has the same epitope specificity as an antigen binding region comprising a heavy chain variable domain comprising or consisting of any one of amino acid sequences selected from SEQ ID NO: 9, 11 and 13 and/or a light chain variable domain comprising or consisting of any one of amino acid sequences selected from SEQ ID NO: 10, 12 and 14.

In another particular embodiment, said anti-CD123 agent can be an immune cell (e.g. T cell) harboring a CAR comprising said first antigen-binding region e.g. scFv, which is immunologically indistinguishable from an antigen binding region comprising as an antigen binding region comprising a heavy chain variable domain comprising or consisting of any one of amino acid sequences selected from SEQ ID NO: 9, 11 and 13 and/or a light chain variable domain comprising or consisting of any one of amino acid sequences selected from SEQ ID NO: 10, 12 and 14.

In a preferred embodiment said anti-CD123 agent can be an immune cell harboring a CAR targeting a specific isoform of CD123 as described in the examples, typically said anti-CD123 CAR comprises a hinge domain, a CD8a transmembrane domain and an intracellular signaling domain from the zeta chain of the CD3 molecule and co-stimulatory domains 4-1BB, preferably said anti-CD123 CAR comprises or consists of sequence SEQ ID NO: 15.

According to the present disclosure, said anti-CD123 agent can be an immune cell harboring an antigen receptor targeting CD123, such as a CAR targeting a specific isoform of CD123, said antigen receptor comprising an antigen binding region as described above and said immune cell either not expresses CD123 or expresses an isoform of CD123 which is not recognized by said CAR.

In specific embodiments, said anti-CD123 agent can be an immune cell (e.g. T cell) harboring a CAR, said CAR targeting a specific isoform of CD123 comprising an antigen-binding region, e.g. scFv, comprising:
a) an antibody heavy chain variable domain (VH) comprising the three CDRs VHCDR1, VHCDR2 and VHCDR3 wherein VHCD1 is SEQ ID NO: 2, VHCD2 is SEQ ID NO: 3 and VHCDR3 is SEQ ID NO: 4; and
b) an antibody light chain variable domain (VL) comprising the three CDRs VLCDR1, VLCDR2 and VLCDR3 wherein VLCDR1 is SEQ ID NO: 5 or 6, VLCDR2 is SEQ ID NO: 7, VLCDR3 is SEQ ID NO: 8;
   and said immune cell either not expresses CD123 or expresses an isoform of CD123 which is not recognized by said CAR.

In a more particular embodiment, said anti-CD123 agent can be an immune cell (e.g. T cell) harboring a CAR comprising said first antigen-binding region e.g. scFv comprising a heavy chain variable domain comprising or consisting of any one of amino acid sequences selected from SEQ ID NO: 9, 11 and 13 and/or a light chain variable domain comprising or consisting of any one of amino acid sequences selected from SEQ ID NO: 10, 12 and 14, and said immune cell expresses an isoform of CD123 which is not recognized by said CAR.

In a more preferred embodiment, said anti-CD123 agent is CSL362 antibody as described in the examples.

In another preferred embodiment, said anti-CD123 agent is MIRG123 antibody as described in the examples.

In another preferred embodiment said anti-CD123 agent can be an immune cell harboring a CAR targeting a specific isoform of CD123 as described in the examples, typically said anti-CD123 CAR comprises a hinge domain, a CD8a transmembrane domain and an intracellular signaling domain from the zeta chain of the CD3 molecule and co-stimulatory domains 4-1BB, preferably said anti-CD123 CAR comprises or consists of sequence SEQ ID NO: 15.

In particular, the disclosure also relates to depleting anti-CD123 agents as disclosed above (for example CAR cell composition or antibodies) comprising a first or a second antigen binding region for use in selectively depleting the host cells or transferred cells respectively, in a subject in need thereof.

### Cell or Population of Cells Expressing a First Isoform of Surface Protein

The present disclosure relates to a mammalian cell, preferably a hematopoietic cell, or a population of cells expressing a first isoform of surface protein wherein said cell or population of cells express a first isoform of a cell surface protein comprising at least one polymorphic allele in the nucleic acid encoding said first isoform, and wherein said first isoform is not recognized by the depleting agent comprising a first antigen binding region as described herein.

Said cell or population of cells are particularly useful in medical treatment in a patient expressing a second isoform of said cell surface protein.

In a particular embodiment, said cells (e.g. hematopoietic stem cell) encoding or expressing said first isoform not recognized by a depleting agent (e.g. hematopoietic cells) are particularly useful in medical treatment to restore normal hematopoiesis after immunotherapy, such as adoptive cell transfer in a patient expressing said second isoform, in particular wherein the treatment comprises administering a therapeutically efficient amount of said hematopoietic cells expressing said first isoform in combination with a therapeutically efficient amount of a depleting agent targeting said second isoform. In particular, said hematopoietic cells, preferably hematopoietic stem cells are administered subsequently to said depleting agent. In another particular embodiment, said hematopoietic cells, preferably hematopoietic stem cells can be administered before or concurrently to said depleting agent

In another particular embodiment, said cells expressing said first isoform specifically recognized by depleting agent which does not bind second isoform are particularly useful in medical treatment in a patient expressing said second isoform, in particular to avoid severe side-effect related to transplanted cells (safety switch), wherein the treatment comprises administering a therapeutically efficient amount of a depleting agent targeting said first isoform. In particular, said hematopoietic cells, preferably immune cells harboring a CAR are administered prior to said depleting agent.

As used herein, the term cell relates to mammalian cells, preferably human cells.

In a particular embodiment, said cells are hematopoietic cells. Hematopoietic cells comprise immune cells including lymphocytes, such as B cells and T cells, natural killer cells, myeloid cells, such as monocytes, macrophages, eosinophils, mast cells, basophils, granulocytes, dendritic cells (DC) and plamacytoid dendritic cells (pDCs).

In a preferred embodiment, said immune cells are T cells. In another preferred embodiment, said immune cells are primary T cells. As used herein, the term “T cell” includes cells bearing a T cell receptor (TCR) or a cell derived from a T cell bearing a TCR. T-cells according to the disclosure can be selected from the group consisting of inflammatory T-lymphocytes, cytotoxic T-lymphocytes, regulatory T-lymphocytes, tumor infiltrating lymphocytes or helper T-lymphocytes included both type 1 and 2 helper T cells and Thl7 helper cells. In another embodiment, said cell can be derived from the group consisting of CD4⁺T-lymphocytes and CD8⁺ T-lymphocytes or non-classical T cells such as MR1 restricted T cells, MAIT cells, NKT cells, gamma delta T cells or innate-like T cells.

T-cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments, T-cells can be obtained from a unit of blood collected from a subject using any number of techniques known to the skilled person. Alternatively, T cells can be differentiated from iPS cells.

In another preferred embodiment, said hematopoietic cells are hematopoietic stem cells. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. Representative human stem cells are CD34⁺ cells. Hematopoietic stem cells can be differentiated from iPS cells or can be harvested from mobilized or not mobilized peripheral blood.

In certain embodiments, the cell is an allogeneic cell which refers to a cell derived from a donor that presents similar HLA, at least similar for some HLA to the person receiving the cell. The donor may be a related or unrelated person. In certain embodiments, the cell is an autologous cell which refers to a cell derived from the same person that is receiving the cell.

Said cells may originate from a healthy donor or from a patient, in particular from a patient diagnosed with cancer or an auto-immune disease or from a patient diagnosed with an infection. Hematopoietic cells can be extracted from blood or derived from stem cells.

A person skilled in the art will choose the more appropriate cells according to the patient or subject to be transplanted.

The disclosure further relates to a composition of cells or a population of cells for use in the therapy as disclosed herein.

### Surface Protein

According to the present disclosure, said cell expresses a first isoform of a surface protein. The surface protein according to the present disclosure is a protein attached to the cell membrane. Said surface protein is also named herein cell surface antigen. In particular, in hematopoietic cell, said surface protein can be a cell surface marker selected from the group consisting of: CD123, CD33, CD7, CD117, CD45, CD135, CLEC12a, CD44, and CD70.

In a particular embodiment, said surface protein is CD123, encoded by interleukin-3 receptor alpha subunit (IL3RA) gene (Gene ID: 3563). IL3RA is a specific subunit of a heterodimeric cytokine receptor which is composed of a ligand specific alpha subunit and a signal transducing beta subunit shared by the receptors for interleukin 3 (IL3), colony stimulating factor 2 (CSF2/GM-CSF), and interleukin 5 (IL5). IL-3 is a multipotent cytokine that promotes the development of hematopoietic progenitors into cells of the erythroid, myeloid and lymphoid lineages. Spliced transcript variants encoding distinct proteins have been found, IL3RA type 1 (NCBI reference: NP_002174.1, 10 Jan. 2021) (SEQ ID NO: 1) and 2 (NCBI reference: NP-001254642.1, 10 Jan. 2021).

In line with the present disclosure, it is also possible to combine additional variants or isoforms of CD123 within the methods and compositions of the present disclosure. Such isoforms may for example include double mutants. Such isoforms may for example also include single and double mutants. The methods and compositions of the present disclosure may also be combined with cells carrying a CD123 knock out, e.g., a permanent knock out or a temporarily knock out (e.g. via CRISPRoff). The methods and compositions of the present disclosure may also be used in the depletion of myeloid cells in solid tumors in order to enhance tumor responses.

The methods and compositions of the present disclosure may also be combined with cells combinations, in particular when said surface protein is CD123 with KO of other targets, i.e. CD33 KO, CD7 KO, CLEC12A, CD44 KO and combination thereof.

The methods and compositions of the present disclosure may also comprise cells expressing first isoform of CD123 (CD123 variants) and other surface protein variants such as CD33 variant, CD7 variant and any combination thereof.

### Polymorphism of Surface Protein Isoform

The cell expressing first isoform of surface protein according to the present disclosure comprises genomic DNA with at least one polymorphic allele in the nucleic acid encoding said surface protein. In particular, said polymorphism induces at least one mutation in surface protein region involved in the binding of a specific agent in comparison to said second isoform.

In a preferred embodiment, said first isoform of surface protein remains functional and retain the capacity of performing the same function as the corresponding wild type isoform within a cell without significant impairment. In particular, said first isoform of CD123 have one or more of the following properties similar to that of the second isoform of CD123:
expression on the cell surface,
retained structure,
IL-3 binding,
Intracellular signaling capacity (e.g. STAT5 phosphorylation/signaling), Induction of cell proliferation of cell lines in response to IL-3, Differentiation in humanized mice into multiple lineages/cell types, pDC function of pDCs isolated from humanized mice, as measured in the functional assays as described in the experimental part.

Said first isoform of CD123 can have the hematopoietic stem cell engraftment capacity or edited hematopoietic stem cell colony formation capacity similar to that of the second isoform of CD123. In more detail, the proliferation of TF-1 cells can be induced by IL-3 and this IL-3 dependent induction of proliferation is blocked by CSL362 or MIRG123. Introducing CD123 variant isoforms into TF-1 cells by appropriate gene editing methods such as HDR allows to determine the proliferation rate of TF-1 cells with each of the CD123 variant isoforms and directly compare it to the IL-3 dependent proliferation of wildtype TF-1 cells. CD123 variant isoforms resulting in equal proliferation rates as wildtype CD123 are regarded as being functionally equivalent.

In particular, when the surface protein is CD123 (IL3RA), the first isoform of CD123 remains functional and is activated by IL-3, a cytokine produced by antigen-activated T cells and can induce IL-3 signaling.

Said polymorphism is preferably within a nucleic acid sequence encoding the surface protein region involved in binding of the first agent, preferably located in the extracellular portion of said surface protein, in particular in a solvent-exposed secondary structure element. More particularly; said polymorphism is within a nucleic acid sequence encoding at least one specific amino acid residue involved in binding of the first agent. Said polymorphism can be a mutation such as a deletion, a substitution and/or insertion of at least 1, 2, 3, 4, 5, 6, 7, 8, 9, 10, 12, or 20 nucleotides. In a particular embodiment, said polymorphism is a single nucleotide polymorphism.

The difference in the sequence of the two isoforms may also be genetically introduced. Also here the sequence difference is preferably within a nucleic acid sequence encoding the surface protein region involved in binding of the first agent, preferably located in the extracellular portion of said surface protein, in particular in a solvent-exposed secondary structure element. More particularly; said sequence difference is within a nucleic acid sequence encoding at least one specific amino acid residue involved in binding of the first agent. Said sequence difference can be a mutation such as a deletion, a substitution and/or insertion of at least 1, 2, 3, 4, 5, 6, 7, 8, 9, 10, 12, 15 or 20 nucleotides. In a particular embodiment, said sequence difference is a single point mutation.

The inventors identified natural polymorphisms in nucleic acid sequence encoding amino acid residues involved in anti-CD123 agent binding as described above, in particular polymorphisms inducing substitution of the residues E51, S59 or R84, preferably E51 or S59 relative to CD123 sequence (SEQ ID NO: 1). Thus, the present disclosure relates to a cell expressing an isoform of CD123, wherein said isoform has a substitution in at least one amino acid residue selected from E51, S59 and R84, preferably E51 or S59 of CD123 (SEQ ID NO: 1).

### Natural Polymorphism

In a particular embodiment, said cell according to the present disclosure is selected from a subject comprising native genomic DNA with at least one natural polymorphism allele, preferably single nucleotide polymorphism (SNP) in the nucleic acid encoding said isoform.

In a particular embodiment, when the surface protein is CD123, cells are selected from a subject that comprises native genomic DNA with at least one natural polymorphism allele, in particular SNP, in a nucleic acid sequence encoding CD123 region involved in anti-CD123 agent binding, preferably located in the extracellular portion of said surface protein, more preferably in a solvent-exposed secondary structure element. More particularly, said polymorphism allele is within a nucleic acid sequence encoding the residues E51, S59 and/or R84 of SEQ ID NO: 1, preferably E51 or S59 of SEQ ID NO: 1. In particular, said polymorphism allele causes a substitution of at least one amino acid residue selected from position E51, S59 and/or R84 of SEQ ID NO: 1, preferably E51 or S59 of SEQ ID NO: 1. Preferably amino acid residue E51 is substituted by an amino acid selected from the group consisting of: K, N, T, S, Q, R, M, G and A, preferably K or T. Also preferably amino acid residue S59 is substituted by an amino acid selected from the group consisting of: I, G, P, E, L, T, K, F, R and Y; preferably P or E. Also preferably, amino acid residue R84 is substituted by an amino acid selected from the group consisting of: T, K, S, Q, N, E, H, L and A.

### Gene Editing Method

In another particular embodiment, said cell expressing first isoform according to the present disclosure is obtained by gene editing, preferably by changing the sequence encoding said surface protein in the patient's native genomic DNA.

The cell can be genetically engineered by introducing into the cell a gene editing enzyme to induce said polymorphism resulting in insertion, deletion and/or substitution of amino acids of surface protein. Said gene editing enzyme targets a nucleic acid sequence, named herein target sequence encoding surface protein region involved in first agent binding as described above. In particular, when said surface protein is CD123, said gene editing enzyme targets a nucleic acid encoding at least one residue in position E51, S59 and/or R84 of SEQ ID NO: 1, preferably in such a way that at least one residue in position E51, S59 and/or R84 of SEQ ID NO: 1 is substituted. Preferably amino residue E51 is substituted by an amino acid selected from the group consisting of: K, N, T, S, Q, R, M, G and A, preferably K or T Also preferably, amino acid residue S59 is substituted by an amino acid selected from the group consisting of: I, G, P, E, L, T, K, F, R and Y; preferably P or E. Also preferably, amino acid residue R84 is substituted by an amino acid selected from the group consisting of: T, K, S, Q, N, E, H, L and A.

Gene editing enzyme may be sequence-specific nuclease, base or prime editor.

The term “nuclease” refers to a wild type or variant enzyme capable of catalyzing the hydrolysis (cleavage) of phosphodiester bonds between nucleotides of a nucleic acid (DNA or RNA) molecule, preferably a DNA molecule. By “cleavage” is intended a double-strand break or a single-strand break event.

The term “sequence-specific nuclease” refers to a nuclease which cleaves nucleic acid in a sequence-specific manner. Different types of site-specific nucleases can be used, such as Meganucleases, TAL-nucleases (TALEN), Zing-finger nucleases (ZFN), or RNA/DNA guided endonucleases like Clustered Regularly Interspaced Short Palindromic Repeats (CRISPR)/Cas system and Argonaute (Review in Li et al., Nature Signal transduction and targeted Therapy, 5, 2020; Guha et al., Computational and Structural Biotechnology Journal, 2017, 15, 146-160).

According to the present disclosure, the nuclease generates a DNA cleavage within a target sequence, said target sequence encodes a surface protein region involved in first agent binding as described above. In particular embodiments, the inventors use CRISPR system to induce a cleavage within a target sequence encoding surface protein region recognized by first agent as described above.

By “target sequence”, it is intended targeting a part of the sequence encoding surface protein region involved in first agent binding as described as described above and/or sequences adjacent to said surface protein region involved in first agent binding, in particular at least one (one or two) sequence of up to 50 nucleotides adjacent to said surface protein region involved in first agent binding, preferably 20, 15, 10, 9, 8, 7, 6 or 5 nucleotides adjacent to said repressor binding site.

CRISPR system involves two or more components, Cas protein (CRISPR-associated protein) and single guide RNA. Cas protein is a DNA endonuclease that uses guide RNA sequence as a guide to recognize and generate double-strand cleavage in DNA that is complementary to the single guide RNA sequence. Cas protein comprises two active cutting sites namely HNH nuclease domain and RuvC-like nuclease domain.

By Cas protein is also meant an engineered endonuclease or a homologue of Cas 9 which is capable of cleaving target nucleic acid sequence. In particular embodiments, Cas protein may induce a cleavage in the nucleic acid target sequence which can correspond to either a double-stranded break or a single-stranded break. Cas protein variant may be a Cas endonuclease that does not naturally exist in nature and that is obtained by protein engineering or by random mutagenesis. The Cas protein can be one type of the Cas proteins known in the art. Non-limiting examples of Cas proteins include Cas1, Cas1B, Cas2, Cas3, Cas4, Cas5, Cas6, Cas7, Cas8, Cas9 (also known as Csn1 and Csx12), Cas1O, Csy1, Csy2, Csy3, Cse1, Cse2, Csc1, Csc2, Csa5, Csn2, Csm2, Csm3, Csm4, Csm5, Cmr1, Cmr3, Cmr4, Cmr5, Cnrr6, Csb1, Csb2, Csb3, Csx17, CsxM, Csx 10, Cs 16, CsaX, Csx3, Cs 1, Csx15, Csf1, Csf2, CsO, Csf4, homologs, orthologs thereof, or modified versions thereof. Preferably Cas protein is Streptococcus pyogenes Cas 9 protein.

Cas is contacted with a guide RNA (gRNA) designed to comprise a complementary sequence of the target sequence to specifically induce DNA cleavage within said target sequence, in particular according to the present disclosure a complementary sequence of a part of target sequence encoding surface protein region recognized by agent as described above.

As used herein, a “guide RNA”, “gRNA” or “single guide RNA” refers to a nucleic acid that promotes the specific targeting or homing of a gRNA/Cas complex to a target nucleic acid.

In particular, gRNA refers to RNA that comprises a transactivating crRNA (tracrRNA) and a crRNA. Preferably, said guide RNA corresponds to a crRNA and tracrRNA which can be used separately or fused together. The complementary sequence pairing with the target sequence recruits Cas to bind and cleave the DNA at the target sequence.

According to the present disclosure, crRNA is engineered to comprise a complementary sequence to a part of a target sequence as described above encoding surface protein region recognized by agent, such that it is capable of targeting said region.

In a particular embodiment, the crRNA comprises a sequence of 5 to 50 nucleotides, preferably 15 to 30 nucleotides, more preferably 20 nucleotides which is complementary to the target sequence. As used herein, the terms “complementary sequence” refers to the sequence part of a polynucleotide (e.g. part of crRNA or tracRNA) that can hybridize to another part of polynucleotides under standard low stringent conditions. Preferentially, the sequences are complementary to each other pursuant to the complementarity between two nucleic acid strands relying on Watson-Crick base pairing between the strands, i.e. the inherent base pairing between adenine and thymine (A-T) nucleotides and guanine and cytosine (G-C) nucleotides. Said gRNA can be designed by any methods known by one of skill in the art in view of the present disclosure.

According to the present disclosure said target sequence encodes surface protein region involved in first agent binding, preferably located in the extracellular portion of said surface protein, more preferably in an extracellular loop in comparison to said second isoform, again more preferably comprising amino acid residues involved in agent binding.

In a preferred embodiment, when surface protein is CD123, said target sequence encodes a CD123 region involved binding of a first agent, such as anti-CD123 agent binding as disclosed above. Preferably said target sequence encodes at least one residue in position E51, S59 and/or R84 of SEQ ID NO: 1, preferably E51 or S59 of SEQ ID NO: 1.

In a particular embodiment, said gRNA may target sequence encoding CD123 region involved in binding of a first agent and particularly comprise one of the sequences described in the Table 1 (gRNA sequence).

TABLE 1
gRNA sequences

| gRNA | Sequence | SEQ ID NO |
|---|---|---|
| gRNA E51K-1 (m1guide1) | TATAGCTCACACACAATTTCTG | 16 |
| gRNA E51K-2 (Cas12a gRNA) | ACACTGGCTATAGCTCACACA | 17 |
| gRNA S59P-1 (m2guide1) | TCTGCGGCTGATAAGATACG | 18 |
| gRNA S59P-2 (m2guide2) | TGATAAGATACGGCCATTTA | 19 |
| gRNA S59P-3 (m2guide3) | CATTTAGTATGAGAGATAAC | 20 |
| gRNA R84E-1 (guide 1) | CTGGTTGATGTGGCAGGCTC | 21 |
| gRNA R84E-2 (guide 2) | AGGCTCACCGGTTGGGTGGT | 22 |
| gRNA R84E-3 (guide 3) | TCACCGGTTGGGTGGTAAGA | 23 |

In another terms, when the surface protein is CD123, said nucleic acid construct preferably can comprise:
a gRNA sequence of: SEQ ID NO: 16 or 17 which targets a sequence encoding the substitution E51K relative to SEQ ID NO: 1,
a gRNA sequence selected from the group consisting of: SEQ ID NO: 18 to 20 which targets a sequence encoding the substitution S59P relative to SEQ ID NO: 1, or
a gRNA sequence selected from the group consisting of: SEQ ID NO: 21 to 23 which targets a sequence encoding the substitution R84E relative to SEQ ID NO: 1.

The DNA strand break that is introduced by the nuclease according to the disclosure can result in mutation of the DNA at the cleavage site via non-homologous end joining (NHEJ) which often results in small insertions and/or deletions or replacement of the DNA surrounding the cleavage site via homology-directed repair (HDR).

In a preferred embodiment, said polymorphism within nucleic acid encoding surface protein isoform is induced via HDR repair following the DNA cleavage and the introduction of an exogeneous nucleotide sequence, named herein HDR template.

HDR template comprises a first and a second portion of sequence which are homologous to regions 5′ and 3′ of the target sequence, respectively and an exogeneous sequence comprising polymorphism. Following cleavage of the target sequence, a homologous recombination event is achieved between the genome containing the target sequence and the HDR template and the genomic sequence containing the target sequence is replaced by the exogeneous sequence.

Preferably, homologous sequences of at least 20 bp, preferably more than 50 bp and more preferably less than 200 bp are used. Indeed, shared DNA homologies are located in regions flanking upstream and downstream the site of the break and the exogeneous sequence to be introduced should be located between the two arms.

In a preferred embodiment, the cell according to the present disclosure is genetically engineered by introducing into said cell said site-specific nuclease which targets the sequence encoding surface protein region recognized by said first agent as described above and a HDR template.

In another particular embodiment, said gene editing enzyme is a DNA base editor as described in Komor et al., Nature 533, 420-424, doi:10.1038/nature17946 and in Rees H A, Liu D R. Nat Rev Genet. 2018 December; 19(12):770-788 or a prime editor as described in Anzalone A V. Et al. Nature, 2019, 576:149-157, Matsoukas I G. Front Genet. 2020; 11: 528 and Kantor A. et al. Int. J. Mol. Sci. 2020, 21(6240). Base editor or prime editor can be used to introduce mutations at specific sites in the target sequence.

According to the present disclosure, the base editor or prime editor generates a mutation within the target sequence by sequence-specific targeting of the sequence encoding surface protein region involved in first agent binding.

In particular, said base editor or prime editor are CRISPR base or prime editors. Said CRISPR base or prime editor comprises as catalytically inactive sequence specific nuclease a dead Cas protein (dCas). dCas refers to a modified Cas nuclease which lacks endonucleolytic activity. Nuclease activity can be inhibited or prevented in dCas proteins by one or more mutations and/or one or more deletions in the HNH and/or RuvC-like catalytic domains of the Cas protein. The resulting dCas protein lacks nuclease activity but bind to a guide RNA (gRNA)-DNA complex with high specificity and efficiency to specific target sequence. In particular embodiment, said dead Cas may be a Cas nickase wherein one catalytic domain of the Cas is inhibited or prevented.

Said base editor is contacted with a guide RNA (gRNA) designed to comprise a complementary sequence of the target nucleic acid sequence to specifically bind said target sequence as described above.

Said gRNA can be designed by any methods known by one of skill in the art in view of the present disclosure. In a particular embodiment, said gRNA may target the sequence encoding the surface protein region recognized by said first agent as described above and in particular when surface protein is CD123, comprises one of the sequences described in the Table 1 (gRNA sequence).

As non-limiting examples said base editor is a nucleotide deaminase domain fused to a dead Cas protein, in particular Cas nickase. Said nucleotide deaminase may be an adenosine deaminase or cytidine deaminase. Said nucleotide deaminase may be natural or engineered deaminase.

In a particular embodiment, said base editor may be as non-limiting examples selected from the group consisting of: BE1, BE2, BE3, BE4, HF-BE3, Sa-BE3, Sa-BE4, BE4−Gam, saBE4-Gam, YE1-BE3, EE-BE3, YE2-BE3, YEE-BE3, VQR-BE3, VRER-BE3, SaKKH-BE3, cas12a-BE, Target-AID, Target-AID-NG, xBE3, eA3A-BE3, A3A-BE3, BE-PLUS, TAM, CRIPS-X, ABE7.9, ABE7.10, ABE7.10* xABE, ABESa, ABEmax, ABE8e, VQR-ABE, VRER-ABE and SaKKH-ABE.

Said prime editor consists of a fusion of a catalytically inactive sequence specific nuclease as described above, particularly a Cas nickase and a catalytically active engineered reverse transcriptase (RT) enzyme. Said fusion protein is used in combination with a prime editing guide RNA (pegRNA) which contains the complementary sequence to the target sequence as described above, particularly when surface protein is CD123 comprises one of the sequences described in the Table 1 and also an additional sequence comprising a sequence that binds to the primer binding site region on the DNA. In particular embodiment, said reverse transcriptase enzyme is a Maloney murine leukemia virus RT enzyme and variants thereof. Said prime editor may be as non-limiting examples selected from the group consisting of: PE1, PE2, PE3 and PE3b.

### CAR

For use in adoptive cell transfer therapy, said cell expressing first isoform according to the present disclosure may be modified to display desired specificities and enhanced functionalities. In particular, the cell may be modified to be directed to a specific target. In a particular embodiment, said cell may express a recombinant antigen binding region, also named antigen receptor on its cell surface as described above. In a particular embodiment, said recombinant antigen receptor is a chimeric antigen receptor (CAR).

According to the present disclosure, said immune cell expressing a first isoform of a cell surface protein and a CAR can be specifically depleted by the administration of a therapeutically efficient amount of an agent which comprises a second antigen binding region which specifically binds to said first isoform but not to the second isoform of said surface protein, thereby avoiding eventual severe side effects due to transplantation of said immune cells.

In a particular embodiment, the immune cell is redirected against a cancer antigen. By “cancer antigen” is meant any antigen (i.e., a molecule capable of inducing an immune response) which is associated with cancer. An antigen as defined herein may be any type of molecule which induces an immune response, e.g., it may be a polysaccharide or a lipid, but most preferably it is a peptide (or protein). Human cancer antigens may be human or human-derived. A cancer antigen may be a tumour-specific antigen, by which is meant an antigen which is not found in healthy cells. Tumour-specific antigens generally result from mutations, in particular frame-shift mutations which generate a wholly new amino acid sequence not found in the healthy human proteome.

Cancer antigens also include tumour-associated antigens, which are antigens whose expression or production is associated with (but not limited to) tumour cells. Examples of tumour-associated antigens include for instance Her2, prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD99, CD117, CD123, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-Dl, muscle-specific actin, neurofilament, neuron-specific enolase (NSE), placental alkaline phosphatase, synaptophysin, thyroglobulin, thyroid transcription factor-1, the dimeric form of the pyruvate kinase isoenzyme type M2 (tumor M2-PK), CD 19, CD22, CD33, CD123, CD27, CD30, CD70, GD2 (ganglioside G2), EGFRvIII (epidermal growth factor variant III), sperm protein 17 (Spl7), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein. In another specific embodiment, said tumor-associated antigen or tumor-specific antigen is integrin avP3 (CD61), galactin, K-Ras (V-Ki-ras2 Kirsten rat sarcoma viral oncogene), or Ral-B.

In a particular embodiment, for use in adoptive cell transfer therapy, preferably for the treatment of malignant hematopoietic disease such as acute myeloid leukemia (AML) or B-acute lymphoblastic leukemia (B-ALL), the immune cell according to the present disclosure expresses a recombinant antigen binding region such as a CAR targeting CD123. Said cell expressing the first isoform and expressing the CAR (e.g. CAR-CD123) can be further specifically depleted by administering a depleting agent comprising a second antigen-binding region which binds specifically to the first isoform of CD123 but does not bind to the second isoform of CD123, thereby avoiding eventual severe side effects such as graft-versus-host disease due to the transplantation.

In specific embodiments, said immune cell (e.g. T cell) expressing the first isoform harbors a CAR targeting CD123, said CAR comprising an antigen-binding region, e.g. scFv, comprising an antigen-binding region which binds specifically to an epitope of CD123 located within the N-terminal domain, or within the polypeptide including the amino acids T48, D49, E51, A56, D57, Y58, S59, M60, P61, A62, V63, N64, T82, R84, V85, A86, N87, P89, F90, S91 of SEQ ID NO: 1.

In particular, said immune cell (e.g. T cell) expressing first isoform harbors a CAR targeting CD123 comprising an antigen-binding region, e.g. scFv, comprising
a) an antibody heavy chain variable domain (VH) comprising the three CDRs VHCDR1, VHCDR2 and VHCDR3 wherein VHCD1 is SEQ ID NO: 2, VHCD2 is SEQ ID NO: 3 and VHCDR3 is SEQ ID NO: 4; and
b) an antibody light chain variable domain (VL) comprising the three CDRs VLCDR1, VLCDR2 and VLCDR3 wherein VLCDR1 is SEQ ID NO: 5 or 6, VLCDR2 is SEQ ID NO: 7, VLCDR3 is SEQ ID NO: 8,
   more preferably comprising an antigen-binding region comprising a heavy chain variable domain comprising or consisting of any one of amino acid sequences selected from SEQ ID NO: 9, 11 and 13 and/or a light chain variable domain comprising or consisting of any one of amino acid sequences selected from SEQ ID NO: 10, 12 and 14.

In a preferred embodiment said anti-CD123 agent can be an immune cell harboring a CAR targeting a specific isoform of CD123 as described in the examples, typically said anti-CD123 CAR comprises a hinge domain, a CD8a transmembrane domain and an intracellular signaling domain from the zeta chain of the CD3 molecule and co-stimulatory domains 4-1BB, preferably said anti-CD123 CAR comprises or consists of sequence SEQ ID NO: 15.

### In Vitro Method for Preparing Cell Expressing First Isoform

The cell expressing the first isoform according to the present disclosure can be genetically engineered by introducing into said cell a nucleic acid construct (e.g. mRNA) encoding at least one gene editing enzyme or ribonucleoprotein complex comprising gene editing enzyme and/or HDR template as described above. Said cell can also be genetically engineered by further introducing into said cell a nucleic acid construct encoding a CAR as described above. In particular, said method is an ex vivo method performed on a culture of cells.

The term “nucleic acid construct” as used herein refers to a man-made nucleic acid molecule resulting from the use of recombinant DNA technology. A nucleic acid construct is a nucleic acid molecule, either single- or double-stranded, which has been modified to contain segments of nucleic acid sequences, which are combined and juxtaposed in a manner, which would not otherwise exist in nature. A nucleic acid construct usually is a “vector”, i.e., a nucleic acid molecule which is used to deliver exogenously created DNA into a host cell.

Preferably, the nucleic acid construct comprises said gene editing enzyme, HDR template and/or CAR, operably linked to one or more control sequences. Said control sequences may be a ubiquitous, tissue-specific or inducible promoter which is functional in cells of target organs (i.e., hematopoietic cell). Such sequences which are well-known in the art include in particular a promoter, and further regulatory sequences capable of further controlling the expression of a transgene, such as without limitation, enhancer, terminator, intron, silencer.

The nucleic acid construct as described above may be contained in an expression vector. The vector may be an autonomously replicating vector, i.e., a vector that exists as an extra-chromosomal entity, the replication of which is independent of chromosomal replication, e.g., a plasmid, an extra-chromosomal element, a mini-chromosome, or an artificial chromosome. The vector may contain any means for assuring self-replication. Alternatively, the vector may be one that, when introduced into the host cell, is integrated into the genome and replicated together with the chromosome(s) into which it has been integrated.

Examples of appropriate vectors include, but are not limited to, recombinant integrating or non-integrating viral vectors and vectors derived from recombinant bacteriophage DNA, plasmid DNA or cosmid DNA. Preferably, the vector is a recombinant integrating or non-integrating viral vector. Examples of recombinant viral vectors include, but not limited to, vectors derived from herpes virus, retroviruses, lentivirus, vaccinia viruses, adenoviruses, adeno-associated viruses or bovine papilloma virus.

The present disclosure relates to a method for expressing a first isoform of a cell surface protein in a cell by introducing into said cell a nucleic acid construct (e.g. mRNA) encoding the gene editing enzyme or ribonucleoprotein complex comprising gene editing enzyme and/or HDR template as described above. Said method may further comprise a step of introducing into said cell a nucleic acid construct encoding a CAR. Said method involves introducing gene editing enzyme such as Cas protein, base editor or prime editor and guide RNA (crRNA, tracrRNa, or fusion guide RNA or pegRNA) into a cell. In particular, said gene editing enzyme is CRISPR/cas gene editing enzyme as described above. In a more particular embodiment, said gene editing enzyme is a site-specific nuclease, more preferably CRISPR/Cas nuclease comprising a guide RNA and Cas protein, wherein said guide RNA in combination with Cas protein cleaves and induces cleavage within said target sequence comprising a nucleic acid encoding surface protein region involved in agent binding as described above.

In a preferred embodiment, said nucleic acid construct comprises CRISPR/Cas nuclease capable of targeting a nucleic acid sequence encoding the surface protein region involved in binding to the depleting agent. When the surface protein is CD123, said nucleic acid construct preferably comprises:
a gRNA sequence of: SEQ ID NO: 16 or 17 which targets a sequence encoding the substitution E51K relative to SEQ ID NO: 1,
a gRNA sequence selected from the group consisting of: SEQ ID NO: 18 to which targets a sequence encoding the substitution S59P relative to SEQ ID NO: 1, or
a gRNA sequence selected from the group consisting of: SEQ ID NO: 21 to 23 which targets a sequence encoding the substitution R84E relative to SEQ ID NO: 1.

Said gene editing enzyme, preferably guide RNA and/or Cas protein, base editor or prime editor as described above may be synthesized in situ in the cell as a result of the introduction of nucleic acid construct, preferably expression vector encoding said gene editing enzyme such as guide RNA and/or Cas protein, base editor or prime editor as described above into the cell. Alternatively, said gene editing enzyme such as guide RNA and/or Cas protein, base editor or prime editor may be produced outside the cell and then introduced thereto.

Said nucleic acid construct or expression vector can be introduced into cell by any methods known in the art and include, as non-limiting examples, stable transduction methods in which the nucleic acid construct or expression vector is integrated into the cell genome, transient transfection methods in which the nucleic acid construct or expression vector is not integrated into the genome of the cell and virus-mediated methods. For example, transient transformation methods include for example microinjection, electroporation, cell squeezing or particle bombardment.

### Pharmaceutical Composition

In a further aspect, the present disclosure also provides a pharmaceutical composition comprising cells or a population of cells expressing a first isoform of a cell surface protein as described above with one or more pharmaceutically or physiologically acceptable carriers, diluents or excipients.

In particular embodiments, said cell expressing said first isoform is an immune cell, preferably a T-cell, more preferably a primary T cell, bearing a chimeric antigen receptor (CAR), preferably a CAR which targets the second isoform of CD123 expressed by said patient's cells as described above.

In another particular embodiment, said cell expressing the first isoform of a cell surface protein is a hematopoietic stem cell.

The pharmaceutical composition may further comprise a depleting agent comprising a first or second antigen binding region as described above.

The pharmaceutical composition is formulated in a pharmaceutically acceptable carrier according to the route of administration. Preferably, the composition is formulated to be administered by intravenous injection. Pharmaceutical compositions suitable for such administration may comprise the cells expressing first isoform as described above, in combination with one or more pharmaceutically acceptable sterile isotonic aqueous or nonaqueous solutions (e.g., balanced salt solution (BSS)), dispersions, suspensions or emulsions, or sterile powders which may be reconstituted into sterile injectable solutions or dispersions just prior to use, which may contain antioxidants, buffers, bacteriostats, solutes or suspending or thickening agents.

Optionally, the composition comprising cells expressing first isoform may be frozen for storage at any temperature appropriate for storage of the cells. For example, the cells may be frozen at about −20° C., −80° C. or any other appropriate temperature. Cryogenically frozen cells may be stored in appropriate containers and prepared for storage to reduce risk of cell damage and maximize the likelihood that the cells will survive thawing. Alternatively, the cells may also be maintained at room temperature of refrigerated, e.g., at about 4° C.

### Therapeutic Use

The present disclosure relates to the cell or population of cells expressing a first isoform as described above for use as a medicament, in particular for use in immunotherapy such as adoptive cell transfer therapy in a patient.

According to the present disclosure, said cell or population of cells (e.g., hematopoietic cells) expressing a first isoform of a cell surface protein as described above (e.g., a first isoform of CD123), is used in a medical treatment in a patient in need thereof, wherein said medical treatment comprises administering a therapeutically efficient amount of cell or population of cells expressing said first isoform of a cell surface protein, in combination with a therapeutically efficient amount of a depleting agent (e.g. a CAR cell or antibody) that binds specifically to the second isoform or first isoform of said cell surface protein (e.g. of CD123) to specifically depleting the patients or the transplanted cells, respectively.

As used herein, the term “in combination” or “in combination therapy” means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. The delivery can be such that an effect of the first treatment delivered is still detectable when the second is delivered. In one embodiment, a depleting agent that binds to a second isoform or a first isoform of a cell surface protein is administered at a dose and/or dosing schedule described herein, and the cells expressing the first isoform are administered at a dose and/or a dosing schedule described herein. In some embodiments, “in combination with,” is not intended to imply that the depleting agent targeting the second (e.g. CAR cells or antibody recognizing a second isoform of CD123) or the first isoform of the cell surface protein and compositions of cells expressing said first isoform of said cell surface protein (e.g., a first isoform of CD123), must be administered at the same time and/or formulated for delivery together, although these methods of delivery are within the scope of this disclosure. The depleting agent (e.g. CAR cells or antibody targeting a second isoform of CD123) can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after) a dose of the hematopoietic stem cells expressing the first isoform of the cell surface protein (e.g. a first isoform of CD123). In certain embodiments, each agent will be administered at a dose and/or on a time schedule determined for that particular agent.

Adoptive cell transfer therapy according to the disclosure can be used to treat patients diagnosed with cancer, autoimmune disease, infectious disease, a disease requiring a hematopoietic stem cell transplantation (HSCT), the prevention of organ rejection, the tumor conditioning regimen, tumor maintenance treatment, minimal residual disease, the prevention of relapse.

The present disclosure also relates to the use of cells expressing a first isoform of a cell surface protein as described above in the manufacture of a medicament for adoptive transfer cell therapy in a patient.

As used herein, the term “subject”, or “patient” refers to an animal, preferably to a mammal in which an immune response can be elicited including human, pig, chimpanzee, dog, cat, cow, mouse, rabbit or rat. More preferably, the subject is a human, including adult, child and human at the prenatal stage.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease.

Cancers that may be treated include tumors that are not vascularized, or not yet substantially vascularized, as well as vascularized tumors. The cancers may comprise non-solid tumors (such as hematological tumors, for example, leukemias and lymphomas including relapses and treatment-related tumors e.g. secondary malignancies after hematopoietic stem cell transplantation (HSCT)) or may comprise solid tumors.

The term “autoimmune disease” as used herein is defined as a disorder that results from an autoimmune response. An autoimmune disease is the result of an inappropriate and excessive response to a self-antigen.

Infectious disease is a disease caused by pathogenic microorganism such as bacteria, viruses, parasites or fungi. In particular embodiments, infections according to the disclosure occur in immunosuppressed patients, such as patients after HSCT or patients who received a solid organ transplantation.

In a preferred embodiment, the present disclosure relates to a cell expressing first isoform of CD123 as described above for use in hematological cancer, preferably leukemia or lymphoproliferative disorders. Said leukemia can be selected from the group consisting of: acute myelogenous leukemia (AML), myelodysplastic syndrome (MDS), blastic plasmacytoid dendritic cell neoplasm (BPDCN), chronic myelogenous leukemia, chronic lymphoid leukemia (CLL), acute biphenotypic leukemia, hairy cell leukemia, interleukin-3 receptor subunit alpha positive leukemia, B-cell acute lymphoblastic leukemia (B-ALL), T-cell acute lymphoblastic leukemia (T-ALL), hodgkin lymphoma (HL), systemic mastocytosis and preferably MDS, preferably AML or BPDCN.

In a particular embodiment, said cell or population of cells (e.g., hematopoietic cells) expressing a first isoform of a cell surface protein as described above (e.g., a first isoform of CD123), can be used for the treatment of solid tumor, in particular for selective depletion of myeloid cells in solid tumors in a patient, to enable immunotherapy agent such as immune checkpoint inhibitors, CAR T-cells or TIL to access to tumors since myeloid cells in tumors can be immunosuppressive. In this situation said cell or population of cells (e.g., hematopoietic cells) expressing a first isoform of a cell surface protein as described above (e.g., a first isoform of CD123), can serve to replenish the hematopoietic system that might be affected by the treatment intended to deplete the myeloid cells in solid tumors.

In another particular embodiment, said cell or population of cells (e.g., hematopoietic cells) expressing a first isoform of a cell surface protein as described above (e.g., a first isoform of CD123) can be used for the treatment of autoimmune disease such as lupus, Multiple sclerosis, Scleroderma.

The disclosure also relates to depleting agents (for example CAR cell composition or antibodies) comprising a first or a second antigen binding region for use in selectively depleting the host cells or transferred cells respectively, in a subject in need thereof.

Method for depleting specifically patient cells and not transplanted cells.

According to the present disclosure, said cell or population of cells (e.g. hematopoietic cells) expressing a first isoform of a cell surface protein as described above (e.g. a first isoform of CD123), is used in a medical treatment in a patient in need thereof, wherein said medical treatment comprises administering a therapeutically efficient amount of said cells or population of cells expressing said first isoform of a cell surface protein, in combination with a therapeutically efficient amount of a depleting agent (e.g. a CAR cell or antibody) that binds specifically to a second isoform of a cell surface protein (e.g. of CD123).

Indeed, during immunotherapy, immunodepleting agent, such as a CAR expressing immune cells directed to a tumoral antigen (e.g., CD123), can be administered to a patient to target and kill tumoral cells. However, as tumoral surface protein are also expressed at the surface of normal hematopoietic cells, this strategy can induce severe side effects to the patients by altering hematopoiesis. To restore hematopoiesis in the patient, hematopoietic cells can be subsequently transplanted into the patient. However, these cells need to be resistant to said agent (e.g., the depleting agent for CD123 expressing cells) in order not to be targeted by it.

Thus, alternatively, according to the present disclosure, the depleting agent comprising a first antigen binding region which binds specifically to a second isoform of a cell surface protein (e.g., of CD123) can be administered to ablate specifically patient cells expressing said second isoform of a cell surface protein (e.g., of CD123) and not transplanted cells expressing said first isoform (e.g., of CD123). The selective depletion of patient cells but not transplanted cells allows to reconstitute the patient with a healthy hematopoietic system which will no longer be depleted by immunodepleting agent. Thus, according to the present therapeutic use, the patients have a functional immune system rather than go through a prolonged phase of immunodepression. The use of cells according to the present disclosure eliminates infections as a major complications of current HSC transplantation.

In another embodiment, the present disclosure relates to a method for adoptive cell transfer therapy, preferably for hematopoietic stem cell transplantation to restore normal hematopoiesis in a patient having cells expressing a second isoform of a surface protein (e.g., CD123) comprising:
(i) administering an effective amount of a cell (e.g. hematopoietic stem cells) expressing a first isoform of said surface protein (e.g. CD123) wherein said cell expressing said first isoform (e.g.; CD123) comprises genomic DNA with at least one polymorphic allele, preferably single nucleotide polymorphism (SNP) allele, or a genetically engineered allele in the nucleic acid encoding said first isoform and wherein said polymorphism is not present in the genome of the patient having cells expressing said second isoform of said surface protein (e.g.; of CD123) or a pharmaceutical composition thereof; and
(ii) administering a therapeutically efficient amount of an agent comprising at least a first antigen-binding region which binds specifically to said second isoform of said surface protein (e.g.; of CD123) and does not bind to said first isoform of said surface protein (e.g. of CD123) to deplete specifically cells expressing said second isoform of said surface protein (patient's cells).

Said cells expressing the first isoform or pharmaceutical compositions thereof are administered to a subject in combination with (e.g., before, simultaneously or following) an agent comprising a first antigen binding region as described above.

In a preferred embodiment, the depleting agent (e.g., CAR cells or antibody targeting a second isoform of CD123) is administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after) a dose of the hematopoietic stem cells expressing a first isoform of said surface protein (e.g., a first isoform of CD123).

By a “therapeutically efficient amount” or “effective amount” is intended a number of cells, in particular hematopoietic stem cells expressing first isoform as described above administered to a subject that is sufficient to constitute a treatment as defined above, in particular restoration of normal hematopoiesis in a patient.

The administration of the cell or pharmaceutical composition according to the present disclosure may be carried out in any convenient manner, including injection, transfusion, implantation or transplantation. The compositions described herein may be administered to a patient subcutaneously, intradermal, intratumorally, intranodally, intramedullary, intramuscularly, by intravenous or intralymphatic injection, or intraperitoneally. In another embodiment, the cells or pharmaceutical compositions of the present invention are preferably administered by intravenous injection. The cells or pharmaceutical compositions of the present invention may be injected directly into a tumor, lymph node, or site of infection.

The administration of the cells or population of cells can consist of the administration of 10⁴-10⁹ cells per kg body weight, preferably 10⁵ to 10⁷ cells/kg body weight including all integer values of cell numbers within those ranges. The dosage administrated will be dependent upon the age, health and weight of the recipient, kind of concurrent treatment, if any, frequency of treatment and the nature of the effect desired. The cells or population of cells can be administrated in one or more doses. Timing of administration is within the judgment of managing physician and depends on the clinical condition of the subject. The cells or population of cells may be obtained from any source, such as a blood bank or a donor. While individual needs vary, determination of optimal ranges of effective amounts of a given cell type for a particular disease or conditions within the skill of the art.

In particular, the disclosure also relates to depleting anti-CD123 agents as disclosed above (for example CAR cell composition or antibodies) comprising a first antigen binding region for use in selectively depleting the host cells in a subject in need thereof.

Method for depleting specifically transplanted cells and not patient cells (safety switch).

According to the present disclosure, said cell or population of cells (e.g. hematopoietic cells) expressing a first isoform of said surface protein as described above (e.g. a first isoform of CD123), is used in a medical treatment in a patient in need thereof, wherein said medical treatment comprises administering a therapeutically efficient amount of a cell or a population of cells expressing said first isoform of said surface protein, in combination with a therapeutically efficient amount of a depleting agent (e.g. a CAR cell or antibody) that binds specifically to said first isoform of said surface protein (e.g. of CD123).

The cell or population of cells, preferably immune cells expressing the first isoform of the present disclosure is particularly used in adoptive transfer cell transfer therapy into a patient. Said transplanted cell expressing said first isoform of said surface protein can be further depleted in patient by administering a therapeutically efficient amount of a depleting agent comprising a second antigen binding region which specifically binds to the first isoform particularly and does not bind to the second isoform of said surface protein expressed by patient's cells to avoid eventual severe side effects such as graft-versus-host disease due to the transplantation. In this case, said agent comprising a second antigen-binding region which binds specifically to said first isoform of said surface protein (expressed by transplanted cell) is administered to deplete specifically transplanted cells and not patient cells. Selective depletion of the transplanted cells constitutes an important safety feature by providing a “safety switch”.

Graft-versus-host disease (GvHD) relates to a medical complication following the receipt of transplanted tissue from a genetically different person. Immune cells in the donated tissue (the graft) recognize the recipient (the host) as foreign. In certain embodiments, the medical condition is graft-versus-host disease caused by hematopoietic stem cell transplantation or adoptive cell transfer therapy wherein immune cells are transferred into patient.

Said side effects can also occur when transplanted cells, particularly immune cells harboring a CAR have severe side effects such as cytokine release syndrome and/or neurotoxicity. In this case, the transplanted cells expressing the first isoform can be eliminated when said cells become malignant or cause any type of unwanted on-target or off-target damage as a safety switch.

The present disclosure relates to a method for adoptive cell transfer therapy in a patient having cells expressing a second isoform of a surface protein (e.g. CD123) comprising:
(i) administering an effective amount of a cell expressing a first isoform of said surface protein (e.g. CD123) wherein said cell expressing said first isoform (e.g; CD123) comprises genomic DNA with at least one polymorphism allele, preferably single nucleotide polymorphism (SNP) allele, or a genetically engineered allele in the nucleic acid encoding said first isoform of said surface protein and wherein said polymorphism is not present in the genome of the patient having cells expressing said second isoform of said surface protein (e.g; of CD123) or a pharmaceutical composition thereof; and
(ii) administering a therapeutically efficient amount of an agent comprising at least a second antigen-binding region which binds specifically to said first isoform of said surface protein (e.g; of CD123) and does not bind to said second isoform of said surface protein (e.g. of CD123) to deplete specifically cells expressing said first isoform.

Said cells expressing the first isoform or pharmaceutical compositions thereof are administered to a subject in combination with (e.g., before, simultaneously or following) an agent comprising a second antigen binding region as described above.

In a preferred embodiment, the depleting agent (e.g. CAR cells or antibody targeting a second isoform of CD123) is administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after) a dose of the hematopoietic stem cells expressing a first isoform (e.g. a first isoform of CD123).

The administration of the cells or pharmaceutical composition according to the present disclosure may be carried out in any convenient manner, including injection, transfusion, implantation or transplantation. The compositions described herein may be administered to a patient subcutaneously, intradermal, intratumorally, intranodally, intramedullary, intramuscularly, by intravenous or intralymphatic injection, or intraperitoneally. In another embodiment, the cells or pharmaceutical compositions of the present invention are preferably administered by intravenous injection. The cells or pharmaceutical compositions of the present invention may be injected directly into a tumor, lymph node, or site of infection.

The administration of the cells or population of cells can consist of the administration of 104-10⁹ cells per kg body weight, preferably 105 to 10⁷ cells/kg body weight including all integer values of cell numbers within those ranges. The dosage administrated will be dependent upon the age, health and weight of the recipient, kind of concurrent treatment, if any, frequency of treatment and the nature of the effect desired. The cells or population of cells can be administrated in one or more doses. Timing of administration is within the judgment of managing physician and depends on the clinical condition of the subject. The cells or population of cells may be obtained from any source, such as a blood bank or a donor. While individual needs vary, determination of optimal ranges of effective amounts of a given cell type for a particular disease or conditions within the skill of the art.

Accordingly, in specific embodiments, the disclosure relates to a depleting agent (e.g. a CAR cell or an antibody) for use in preventing or reducing the risk of severe side effects in a patient having received a cell expressing a first isoform of a cell surface protein as described above, wherein said patient have native cells expressing a second isoform of said cell surface protein, and wherein said depleting agent comprising at least a second antigen-binding region which binds specifically to said first isoform of said cell surface protein and does not bind to said second isoform of said cell surface protein.

### Kit

In another aspect, the present disclosure relates to a kit for expressing a first isoform of a surface protein as describe above into a cell, said kit comprising a gene editing enzyme, such as guide RNA in combination with a Cas protein, base editor or prime editor, nucleic acid construct, expression vector as described above or isolated cell according to the present disclosure.

The invention is disclosed in the following claims:
1. A mammalian cell or a population of cells expressing a first isoform of a surface protein for use in a medical treatment in a patient in need thereof, said patient having cells expressing a second isoform of said surface protein,
   wherein said cell expressing said first isoform comprises genomic DNA with at least one polymorphism or genetically engineered allele, preferably wherein said polymorphism is a single nucleotide polymorphism (SNP) allele, in the nucleic acid encoding said first isoform, wherein said polymorphism allele is not present in the genome of the patient having cells expressing said second isoform of said surface protein.
2. The mammalian cell or population of cells for use according claim 1, wherein said first and second isoform of surface protein are functional.
3. The mammalian cell or population of cells for use according to claim 1 or 2 wherein said surface protein is CD123.
4. The mammalian cell or population of cells for use according to claim 3 wherein said polymorphic or genetically engineered allele is characterized by at least one substitution of an amino acid in position E51, S59 and/or R84 relative to SEQ ID NO: 1.
5. The mammalian cell or population of cells for use according to claim 4 wherein said residue E51 is substituted by an amino acid selected from the group consisting of: K, N, T, R, M, G and A, preferably K or T.
6. The mammalian cell or population of cells for use according to claim 4 or 5 wherein said residue S59 is substituted by an amino acid selected from the group consisting of: I, P, E, L, K, F, R and Y; preferably P, E.
7. The mammalian cell or population of cells for use according to any one of claims 4 to 6 wherein said residue R84 is substituted by an amino acid selected from the group consisting of: T, S, Q, N, H and A.
8. The mammalian cell or population of cells for use according to any one of claims 1 to 7, wherein said cell expressing said first isoform has been selected from a subject comprising native genomic DNA with at least one natural polymorphism allele in nucleic acid encoding said first isoform.
9. The mammalian cell or population of cells for use according to any one of claims 1 to 7, wherein said first isoform is obtained by ex vivo modifying the nucleic acid sequence encoding said surface protein by gene editing.
10. The mammalian cell or population of cells for use of claim 9 wherein said nucleic acid sequence encoding surface protein is modified by introducing into a cell a gene editing enzyme capable of inducing site-specific mutations(s) within a target sequence encoding surface protein region involved in the binding of agent comprising at least a first antigen-binding region.
11. The mammalian cell or population of cells for use of claim 10 wherein said gene editing enzyme is a site-specific nuclease, base editor or prime editor, preferably a CRISPR/Cas gene editing enzyme comprising a guide RNA which comprises a complementary sequence to said target sequence.
12. The mammalian cell or population of cells for use according to claim 11 wherein said surface protein is CD123 and wherein the
guide RNA sequence is SEQ ID NO: 16 or 17 and targets a sequence encoding the substitution E51K relative to SEQ ID NO: 1,
guide RNA sequence is selected from the group consisting of SEQ ID NO: 18 to 20 and targets a sequence encoding the substitution S59P relative to SEQ ID NO: 1 and/or
guide RNA sequence is selected from the group consisting of SEQ ID NO: 21 to 23 and targets a sequence encoding the substitution R84E relative to SEQ ID NO: 1.
13. The mammalian cell for use according to any one of claims 9 to 12 wherein said nucleic acid sequence encoding said surface protein is modified by further introducing an HDR template.
14. The mammalian cell or population according to any one of claims 1 to 13 wherein said medical treatment comprises:
   administering a therapeutically efficient amount of said cell or population of cells expressing said first isoform to said patient in need thereof, in combination with a therapeutically efficient amount of a depleting agent comprising at least a first antigen-binding region that binds specifically to said second isoform to specifically deplete patient cells expressing second isoform.
15. The mammalian cell or population of cells for use of claim 14 wherein said cell expressing said first isoform is a hematopoietic cell, preferably a hematopoietic stem cell.
16. The mammalian cell or population of cells for use according to claim 14 or to restore normal haematopoiesis after immunotherapy in the treatment of hematopoietic disease, preferably malignant hematopoietic disease such as acute myeloid leukemia (AML), blastic plasmacytoid dendritic cell neoplasm (BPDCN), or B-acute lymphoblastic leukemia (B-ALL).
17. The mammalian cell or population of cells for use according to any one of claims 14 to 16 wherein said depleting agent is an antibody or antibody-drug conjugate comprising a first antigen-binding region which binds specifically to said second isoform and does not bind to said first isoform.
18. The mammalian cell or population of cells for use according to claims 14 to 16 wherein said depleting agent is an immune cell, preferably a T-cell, bearing a chimeric antigen receptor (CAR) comprising a first antigen-binding region which binds specifically to said second isoform and does not bind to said first isoform.
19. The mammalian cell or population of cells for use according to claim 17 or 18 wherein said surface protein is CD123 and wherein said first antigen-binding region of said depleting agent binds specifically to an epitope including the amino acids T48, D49, E51, A56, D57, Y58, 559, M60, P61, A62, V63, N64, T82, R84, V85, A86, N87, P89, F90, S91 of SEQ ID NO: 1.
20. The mammalian cell or population of cells for according to claim 19 wherein said first antigen-binding region comprises:
   c) an antibody heavy chain variable domain (VH) comprising the three CDRs VHCDR1, VHCDR2 and VHCDR3 wherein VHCD1 is SEQ ID NO: 2, VHCD2 is SEQ ID NO: 3 and VHCDR3 is SEQ ID NO: 4; and
   d) an antibody light chain variable domain (VL) comprising the three CDRs VLCDR1, VLCDR2 and VLCDR3 wherein VLCDR1 is SEQ ID NO: 5 or 6, VLCDR2 is SEQ ID NO: 7, VLCDR3 is SEQ ID NO: 8.
21. The mammalian cell or population of cells for according to claim wherein said first antigen binding region comprises a heavy chain variable domain comprising or consisting of any one of amino acid sequences selected from SEQ ID NO: 9, 11 and 13 and/or a light chain variable domain comprising or consisting of any one of amino acid sequences selected from SEQ ID NO: 10, 12 and 14.
22. The mammalian cell or population of cells according to any one of claims 1 to 13 wherein said medical treatment comprises:
   administering a therapeutically efficient amount of said cell or population of cells expressing said first isoform to said patient in need thereof in combination with a therapeutically efficient amount of a depleting agent comprising at least a second antigen-binding region that binds specifically to said first isoform to specifically deplete transferred cells expressing first isoform.
23. The mammalian cell or population of cells for use according to claim 22 in adoptive cell transfer therapy, preferably for the treatment of malignant hematopoietic disease such as acute myeloid leukemia (AML), blastic plasmacytoid dendritic cell neoplasm (BPDCN), or B-acute lymphoblastic leukemia (B-ALL).
24. The mammalian cell or population of cells of claim 22 or 23 wherein said depleting agent is administered subsequently to said cell or population of cells expressing said first isoform of surface protein to avoid eventual severe side effects such as graft-versus-host disease due to the transplantation.
25. The mammalian cell or population of cells for use according to any one of claims 22 to 24 wherein said cell expressing said first isoform is a hematopoietic cell, preferably an immune cell, more preferably a T-cell.
26. The mammalian cell or population of cells for use according to claim 25 wherein said immune cell expressing said first isoform is an immune cell, preferably a T-cell, bearing a chimeric antigen receptor (CAR).
27. The mammalian cell or population of cells for use according to claim 26, wherein said chimeric antigen receptor (CAR) targets the second isoform expressed by said patient's cells.
28. The mammalian cell or population of cells for use according to claim 27 wherein said CAR comprises an antigen-binding region which binds specifically to an epitope of CD123 located within the third extracellular loop, or within the polypeptide including the amino acids T48, D49, E51, A56, D57, Y58, 559, M60, P61, A62, V63, N64, T82, R84, V85, A86, N87, P89, F90, S91 of SEQ ID NO: 1.
29. The mammalian cell or population of cells for use according to claim 28 wherein said CAR comprises an antigen-binding region comprising:
an antibody heavy chain variable domain (VH) comprising the three CDRs VHCDR1, VHCDR2 and VHCDR3 wherein VHCD1 is SEQ ID NO: 2, VHCD2 is SEQ ID NO: 3 and VHCDR3 is SEQ ID NO: 4; and
an antibody light chain variable domain (VL) comprising the three CDRs VLCDR1, VLCDR2 and VLCDR3 wherein VLCDR1 is SEQ ID NO: 5 or 6, VLCDR2 is SEQ ID NO: 7, VLCDR3 is SEQ ID NO: 8.
30. The mammalian cell or population of cells for use according to claim 29 wherein said CAR comprises an antigen-binding region comprising a heavy chain variable domain comprising or consisting of any one of amino acid sequences selected from SEQ ID NO: 9, 11 and 13 and/or a light chain variable domain comprising or consisting of any one of amino acid sequences selected from SEQ ID NO: 10, 12 and 14, preferably wherein said CAR comprises or consists of amino acid sequence SEQ ID NO: 15.
31. The mammalian cell or population of cells for use according to any one of claims 22 to 30 wherein said depleting agent is an antibody or antibody-drug conjugates comprising a second antigen-binding region which binds specifically to said first isoform and does not bind to said second isoform.
32. The mammalian cell or population of cells for use according to claims 22 to 30 wherein said depleting agent is an immune cell, preferably a T-cell, bearing a chimeric antigen receptor (CAR) comprising a second antigen-binding region which binds specifically to said first isoform and does not bind to said second isoform.
33. A pharmaceutical composition comprising a mammalian cell, preferably a hematopoietic stem cell or an immune cell such as T-cell as defined in any one of claims 1 to 13 and a pharmaceutically acceptable carrier.
34. The pharmaceutical composition of claim 33 wherein said immune cell expressing said first isoform is an immune cell, preferably a T-cell, bearing a chimeric antigen receptor (CAR), preferably a CAR which targets the second isoform of CD123 expressed by said patient's cells.
35. The pharmaceutical composition of claim 34 wherein said CAR comprises an antigen-binding region which binds specifically to an epitope of CD123 located within the third extracellular loop, or within the polypeptide including the amino acids T48, D49, E51, A56, D57, Y58, 559, M60, P61, A62, V63, N64, T82, R84, V85, A86, N87, P89, F90, S91 of SEQ ID NO: 1.
36. The pharmaceutical composition of claim 35 wherein said CAR comprises an antigen-binding region comprising:
an antibody heavy chain variable domain (VH) comprising the three CDRs VHCDR1, VHCDR2 and VHCDR3 wherein VHCD1 is SEQ ID NO: 2, VHCD2 is SEQ ID NO: 3 and VHCDR3 is SEQ ID NO: 4; and
an antibody light chain variable domain (VL) comprising the three CDRs VLCDR1, VLCDR2 and VLCDR3 wherein VLCDR1 is SEQ ID NO: 5 or 6, VLCDR2 is SEQ ID NO: 7, VLCDR3 is SEQ ID NO: 8.
37. The pharmaceutical composition of claim 36 wherein said CAR comprises an antigen-binding region comprising a heavy chain variable domain comprising or consisting of any one of amino acid sequences selected from SEQ ID NO: 9, 11 and 13 and/or a light chain variable domain comprising or consisting of any one of amino acid sequence selected from SEQ ID NO: 10, 12 and 14, preferably wherein said CAR comprises or consists of SEQ ID NO: 15.
38. The pharmaceutical composition according to any one of claims 33 to 37 further comprising a depleting agent as defined in claims 27 to 27, 31 and 32.
39. A depleting agent for use in preventing or reducing the risk of severe side effects in a patient having received a cell expressing a first isoform of a surface protein, wherein said patient's native cells express a second isoform of surface protein, and wherein said depleting agent comprises at least a second antigen-binding region which binds specifically to said first isoform and does not bind to said second isoform.
40. The depleting agent for use according to claim 39 wherein said depleting agent is an antibody or antibody-drug conjugates comprising a second antigen-binding region which binds specifically to a first isoform of a surface protein and does not bind to a second isoform of said surface protein.
41. The depleting agent for use according to claim 40 wherein said depleting agent is an immune cell, preferably a T-cell, bearing a chimeric antigen receptor (CAR) comprising a second antigen-binding region which binds specifically to said first isoform and does not bind to said second isoform.
42. The depleting agent for use according to any one of claims 39 to 41 wherein said surface protein is CD123.
43. A depleting agent for use in selectively depleting the host cells in a patient in need thereof wherein said patient's native cells express a second isoform of a surface protein and wherein said depleting agent comprises at least a first antigen-binding region which binds specifically to said second isoform.
44. The depleting agent for use according to claim 44 wherein said depleting agent is an antibody or antibody-drug conjugates comprising a first antigen-binding region which binds specifically to a second isoform of said surface protein.
45. The depleting agent for use according to claim 44 wherein said depleting agent is an immune cell, preferably a T-cell, bearing a chimeric antigen receptor (CAR) comprising a first antigen-binding region which binds specifically to said second isoform.
46. The depleting agent according to any one of claims 44 to 46 wherein said surface protein is CD123.
47. The depleting agent for use according to claim 47 wherein said first antigen-binding region of said depleting agent binds specifically to an epitope located within the third extracellular loop, or within the polypeptide including the amino acids T48, D49, E51, A56, D57, Y58, 559, M60, P61, A62, V63, N64, T82, R84, V85, A86, N87, P89, F90, S91 of SEQ ID NO: 1
48. The depleting agent for use according to claim 48 wherein said first antigen-binding region comprises:
   d) an antibody heavy chain variable domain (VH) comprising the three CDRs VHCDR1, VHCDR2 and VHCDR3 wherein VHCD1 is SEQ ID NO: 2, VHCD2 is SEQ ID NO: 3 and VHCDR3 is SEQ ID NO: 4; and
   f) an antibody light chain variable domain (VL) comprising the three CDRs VLCDR1, VLCDR2 and VLCDR3 wherein VLCDR1 is SEQ ID NO: 5 or 6, VLCDR2 is SEQ ID NO: 7, VLCDR3 is SEQ ID NO: 8.
49. The depleting agent for use according to claim 49 wherein said first antigen binding region comprises a heavy chain variable domain comprising or consisting of any one of amino acid sequences selected from SEQ ID NO: 9, 11 and 13 and/or a light chain variable domain comprising or consisting of any one of amino acid sequence selected from SEQ ID NO: 10, 12 and 14, preferably wherein said depleting agent is a CAR comprising or consisting of SEQ ID NO: 15.

The invention will now be exemplified with the following examples, which are not limitative.

### Examples

### 1. Computational Analysis of CD123

The identity and localisation of the epitope on the cell surface protein can be extracted from the 3D structure of the complex or predicted based on sequence and/or structure-based epitope prediction tools. Methods for predicting linear or conformational B-cell epitopes include but are not limited to BepiPred PMID: 28472356, DiscoTope PMID: 26424260, ElliPro PMID: 19055730, and SVMTriP PMID: 32162263.

To estimate the effect of the variants on the structural and functional status of the protein and possible pathogenic effects, information including biophysical features, evolutionary conservated patterns, proximity to biologically relevant sites, i.e., interaction sites, functionally and structurally relevant sites, protein stability are considered.

The location of the variant on the protein is mapped based on i) available or predicted 3D structure of the protein; ii) sequence comparison to homologous proteins of known structures. Solvent accessibility, or accessible surface area (ASA), of the amino acid site and overall protein region containing the variant is computed based on 3D structure information, when available, or predicted from sequence-based features.

Methods to predict ASA from the amino acid sequence include sequence profiles, structural similarity and machine-learning approaches, such as neural networks, support vector machines (SVM) and Bayesian statistics PMID: 2217139, PMID: 7892171, PMID: 15281128, PMID: 15814555, PMID: 8727318. Methods for predicting the effect of variants include but are not limited to SIFT, PolyPhen, MutationTaster, Condel, FATHMM.

Structure-based methods for predicting the impact of amino acid variations on protein stability are based on statistical potentials, physical or empirical energy-functions, and include but not limited to: FoldX, Rosetta, CC/PBSA (PMID: 12079393, PMID: 18410248, PMID: 19116609, PMID: 15063647).

Global and local probabilistic models computed from multiple sequence alignments can be used to quantify the effect of single or higher-order substitutions from sequence information alone, and include Mutual information, Direct coupling analysis and covariance estimation PMID: 28092658, PMID: 22101153, PMID: 23458856, PMID: 24573474.

### 2. Analysis of CD123-CSL362 Complex

### Csl362 Epitope: Open Vs Closed State

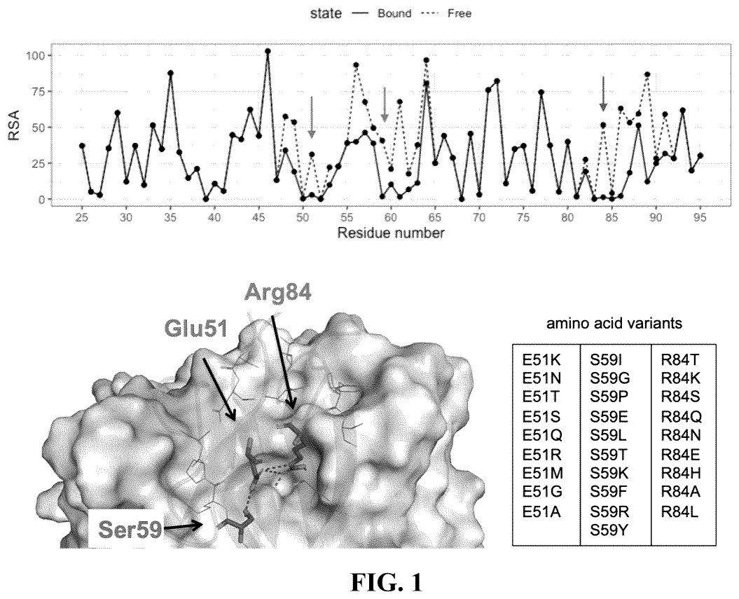

The three-dimensional structure of the CD123-CSL362 interaction complex was used to identify key interaction sites as preferential sites for the generation of protein variants. Protein sites and variants were selected based on: i) per-residue relative solvent accessibility observed in the CSL362-bound and CSL362-free states, ii) per-site evolutionary conservation and amino acid usage from multiple sequence alignments; iii) predicted stability change after in silico mutagenesis based on sequence (EVmutation) and structure-based simulations (FoldX) (FIG. 1).

### 3. SNP Analysis for CD123

Together with the nature of the amino acid change, each variant will be analyzed and prioritized based on the distribution or the frequency of the allele in a given population, homozygote state, disease-association data, phenotype-association data, as well as the effect(s) of the variation at the protein, cell and organism level.

Allele frequency data can be retrieved from a range of projects and repositories including the 1000 Genomes Project; the genome Aggregation Database (gnomAD); dbSNP; Ensembl; Uniprot (Table 1);

Phenotype data and disease-associated variants can be retrieved from a range of projects and repositories including ClinVar; COSMIC phenotype variants; HGMD-PUBLIC variants; NHGRI-EBI catalog of phenotype variants; OMIM phenotype variants; PhenCode; GWAS; Genotype-Tissue Expression (GTEx).

TABLE 1
CD123 SNPs. Data refer to SNPs resulting in non-synonymous variants
at amino acid residues 51, 59 and 84. Source: gnomAD v2.1.1.

| Chromosome | | Reference | Alternate | Protein | Total number | Allele |
|---|---|---|---|---|---|---|
| Position | rsIDs | allele | allele | Consequence | of Genotypes | Frequency |
| X: 1464295 | rs189735318 | G | A | p.Glu51Lys | 250792 | 3.59e−5 |
| X: 1464295 | rs189735318 | G | C | p.Glu51Gln | 282020 | 3.19e−5 |
| X: 1464297 | rs144300259 | G | C | p.Glu51Asp | 281874 | 1.06e−5 |
| X: 1464320 | rs1318699562 | C | G | p.Ser59Cys | 247760 | 4.04e−6 |
| X: 1467391 | rs138837148 | G | C | p.Arg84Pro | 251178 | 3.98e−6 |
| X: 1467391 | rs138837148 | G | A | p.Arg84Gln | 282560 | 1.06e−4 |

### 4. FACS Scanning on Live Cells

### Expression and Purification of CSL362/Okt3

The plasmid encoding the CSL362/Okt3 BiTE is a kind gift from Dario Neri (Hutmacher et aL, Leuk Res. 2019 September; 84:106178).

CHO—S have been grown in Power CHO2medium (Lonza: BELN12-771Q, supplemented with 1XHT, Glutamax, antibiotic-antimycotic) to a density of 20 million per ml.

2.10⁹ cells are then centrifuged, resuspended in 500 ml ProCHO4 medium (Lonza: BEBP12-029Q supplemented with 1XHT, Glutamax, antibiotic-antimycotic). 1.7 mg CSL362/Okt3 BiTE DNA is added together with 5 ml PEI (at lmg/ml) to the cells. Cells are then distributed in 4×500 ml roller bottles, and let grown for 6 days at 31° C., 140 rpm, in a CO2 incubator.

CHO—S cells are then centrifuged for 20 mns at 3000 rpm. Supernatant is filtered through a 0.22 mm filter and applied to a 5 ml Ni NTA column (ThermoFisher) prewashed with 100 ml washing solution (PBS containing 150 mM NaCl and 5 mM imidazole, pH7.4).

Column is washed in 100 ml washing medium. Elution is done in PBS, 150 mM NaCl, 250 mM Imidazole, pH=8.0. 0.5 ml fractions are collected and OD is measured at 280 mM. High concentration fractions are pooled and dialyzed twice O/N against PBS. Bite is sterile filtered through a 0.22 μm, aliquoted at 1 mg/ml, and stored at −80° C.

### CSL362 and MIRG123

CSL362 is a chimeric antibody carrying S239D and 1332E mutations in the Fc region (Leukemia (2014) 28: 2213-21). To generate the monoclonal IgGI anti-CD123 antibody MIRG123, the heavy and kappa light chain variable regions (VH and VKL) were derived from the CSL362/OKT3 BiTE. The VH and VKL sequences were cloned into AbVec2.0-IGHG1 (Addgene plasmid #80795) and AbVec1.1-IGKC (Addgene plasmid #80796), respectively, under the control of a hCMV promoter (Tiller et al J Immunol Methods. 2008 Jan. 1; 329(1-2):112-24. Epub 2007 Oct. 31).

VH sequence (SEQ ID NO: 9):
EVQLVQSGAEVKKPGESLKISCKGSGYSFTDYYMKWARQMPGKGLEWMG
DIIPSNGATFYNQKFKGQVTISADKSISTTYLQWSSLKASDTAMYYCAR
SHLLRAS WFAYWGQGTMVTVSS
VKL sequence
(SEQ ID NO: 10)
DIVMTQSPDSLAVSLGERATINCESSQSLLNSGNQKNYLTWYQQKPGQP
PKPLIYWASTRESGVPDRFSGSGSGTDFTLTISSLQAEDVAVYYCQNDY
SYPYTFGQGTKLEIK

Both plasmids are then co-transfected in CHO—S cells for expression. CHO—S have been grown in Power CHO2 medium (Lonza: BELN12-771Q, supplemented with glutamax, HT supplements, Antibiotic-antimycotic), to a density of 20 million per ml.

2×10⁹ cells are then centrifuged, resuspended in 500 ml ProCHO4 medium (Lonza: BEBP12-029Q supplemented with 1XHT, Glutamax, antibiotic-antimycotic). 0.6 mg VH and 0.6 mg VKL DNA are added together with 5 ml PEI (at 1 mg/ml) to the cells. Cells are then distributed in 4×500 ml roller bottles, and let grown for 6 days at 31° C., 140 rpm, in a CO2 incubator.

CHO—S cells are then centrifuged for 20 mns at 3000 rpm. Supernatant is filtered through a 0.22 μm filter and applied on a protein A column prewashed with PBS.

Column is then washed with 100 ml PBS. Antibody is eluted with 0.1M glycine pH=2.2. 0.5 ml fractions are collected and OD is measured at 280 mM. High concentration fractions are pooled and dialyzed twice O/N against PBS.

### 5. Binding Assays by FACS

### 5.1 Material and Methods

### Eukaryotic Cell Lines

The human embryonic kidney 293 cell line (HEK-293) was a kind gift of M. Zavolan (Biozentrum Basel). All cell lines were freshly thawed and passaged 3-6 times prior to use in assay. HEK-293 were cultured in Dulbecco's Modified Eagle's Medium—high glucose (Sigma-Aldrich) supplemented with 10% heat-inactivated Fetal Calf Serum (FCS; Gibco Life Technologies) and 2 mM Glutamax at 37° C. with 5% CO2 and split three times a week. To the culture medium of all stable cell lines expressing a neomycin resistance cassette Geneticin G418 (50 mg/ml; BioConcept) was added at concentration of 350 μg/ml.

Wildtype HEK-293 negatively stained for CD123 are named as HEK, HEK-293 expressing wildtype CD123 are labelled HEK-CD123, and the CD123 variants are described by the residue's position and the changed amino acids.

Full length cDNA of Human interleukin 3 receptor (CD123), (NM_002183.2) was purchased from Sino Biological, in a pCMV3 vector (catalogue number: HG10518-NF). Hygromycin was replaced by Neomycin for stable cell line generation.

E51 has been mutated to: K, N, T, S, Q, R, M, G, A.

S59 has been mutated to: I, G, P, E, L, T, K, F, R, Y.

R84 has been mutated to: T, K, S, Q, N, E, H A, L.

All point mutations have been then introduced by PCR, leading to single amino acid changes.

2.10⁶ HEK cells are electroporated with 6 μg DNA (pCMV plasmid-huCD123 wt or variants), using the Neon electroporator (Invitrogen; 1100V-20 ms-2 pulses). After 48 hours, cells are used for binding analysis by FACS, as transient transfections. They are then kept 2 weeks under G418 selection (350 μg/ml medium) and then tested as stable cell lines for binding.

FACS:

2×10⁵ HEK cells are stained with anti HuCD123-Buv450 clone 6H6 (Biolegend-Cat:306020-1/100) together with MIRG123− Biotin (1/50)+Streptavidin-FITC (1/200) and analyzed by FACS.

### Flow Cytometry-Based Binding Assay:

In order to test binding of CD123 variants to MIRG123, stable cell lines are stained with high antibody concentration: anti HuCD123-Buv450 clone 6H6 (1/12: 4.2 mg/ml) and biotinylated MIRG123− Bio (1/7.5: 50 mg/ml)+Streptavidin-PE (1/200).

### 5.2 Results

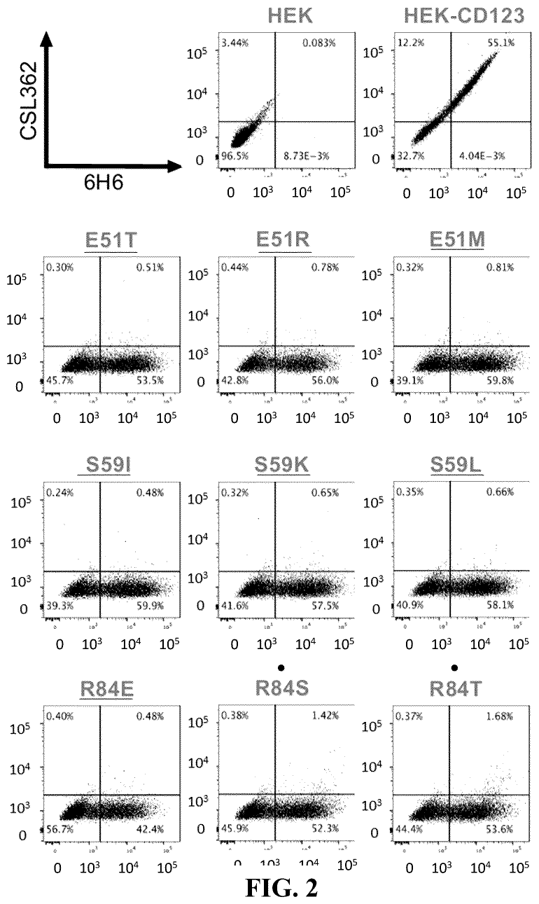

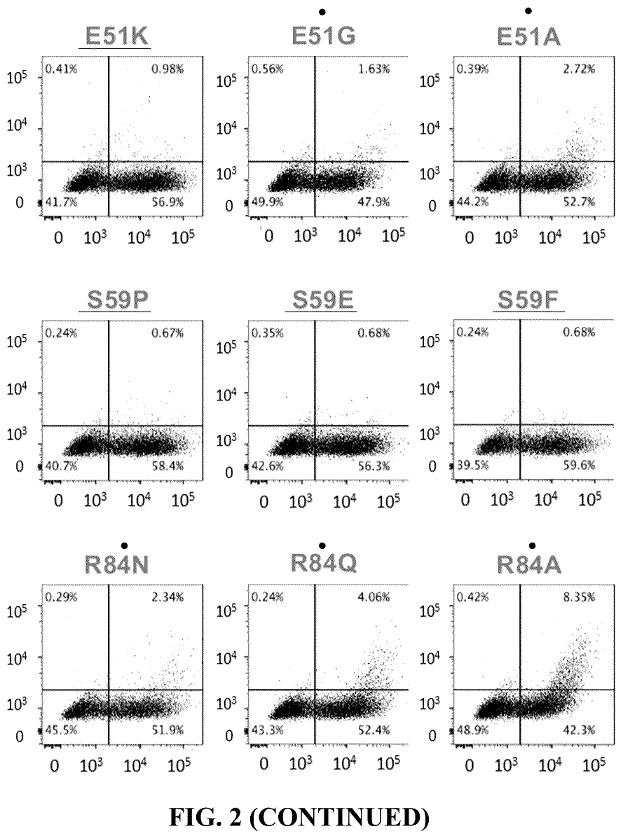

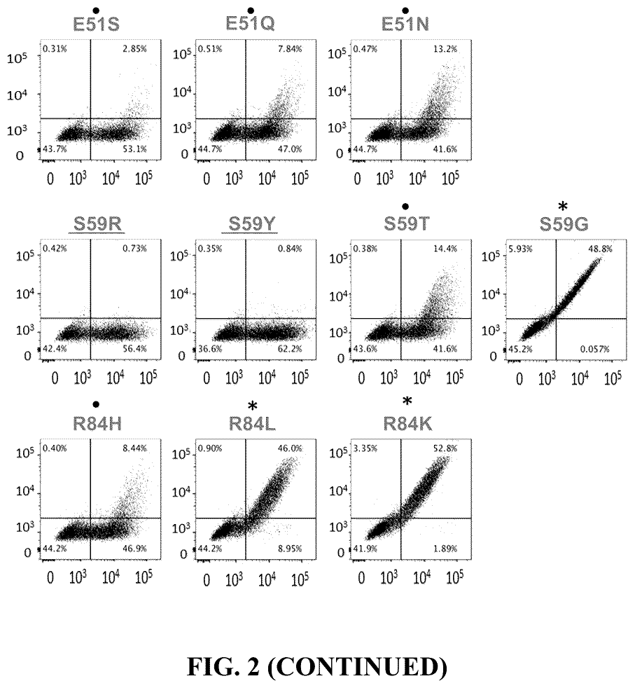

FIG. 2 shows Flow cytometry plots showing binding of the anti-human CD123 antibody clones 6H6 (x-axis) and MIRG123 (y-axis) to wild-type CD123 and its variants stably expressed in HEK-293 cells in vitro. Strong staining with 6H6 indicates stable expression of the CD123 protein, whereas the different CD123 variant isoforms show abolished (<1 MIRG123⁺ 6H6⁺ cells (upper-right quadrant)), weak (1-20% MIRG123⁺ 6H6⁺ cells) or strong (>20% MIRG123⁺6H6⁺ cells) binding to the clone MIRG123. Control conditions (grey) are HEK-293 cells stably expressing human wildtype CD123 (HEK-CD123) and non-transduced HEK-293 cells (HEK). Representative flow cytometry data of 3 independent experiments.

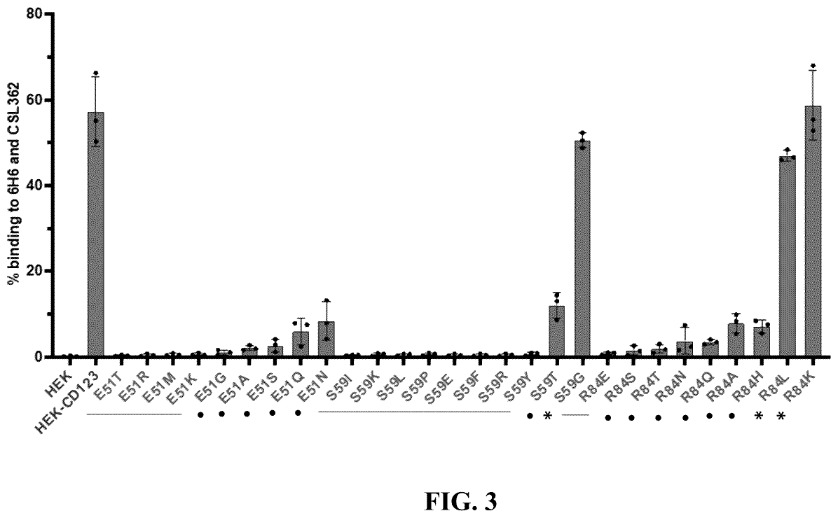

Target cells are stable HEK-293 cell lines, either non-transduced (HEK), expressing wildtype CD123 (HEK-CD123) or its variant isoform (indicated residue position with changed amino acid). The CD123 variants are coded according to their abolished (underlined), weak (circle) or strong (asterix) binding to the anti-CD123 antibody clone MIRG123. Compiled data of 3 independent flow cytometry experiments are shown in FIG. 3.

### 6. ADCC Assay: In Vitro Assay to Quantify ADCC Activity Mediated by MIRG123

### 6.1 ADCC Assay Method

ADCC activities were extrapolated from an Fc□RIIIa activation assay performed with the ADCC Reporter Bioassays, V Variant (Promega, ref G7015). HEK293 cell lines stably expressing the hCD123 variants were seeded in white 96 well-plate clear bottom (Corning costar #3610). At day 0, 4400 cells were seeded in 100 ul culture medium in order to get 6250 cells 24 h later (E:T ratio 12:1). At day 1, medium was removed from the HEK293 cell culture and were added: (i) 25 μl of ADCC buffer (Promega), (ii) 25 μl of MIRG123 antibody diluted into ADCC buffer at 3× the final concentration (final concentration 1 ug/ml), (iii) 25 μl of Effector cells (Jurkat/FcγRIIIa/NFAT-Luc cells) diluted in ADCC buffer at the concentration 3M cells/ml. The mixture was incubated 5 h at 37° C. 5% CO2. Luciferase activity was measured using the Bio-Glo™ Luciferase Assay Reagent (Promega). 75 μl of Reagent were added and cells were incubated 10 min at RT with agitation. An opaque label (Elmer 6005199) was used to cover the bottom of the plate and the luminescence was read with the PHERAstart FSX (BMG LABTECH) program Luc-Glo (LUM), GainA=3600, Optic module=LUMplus.

In this assay Raji cells incubated with 1 μg/ml of rituximab were used as positive control.

### 6.2 Results

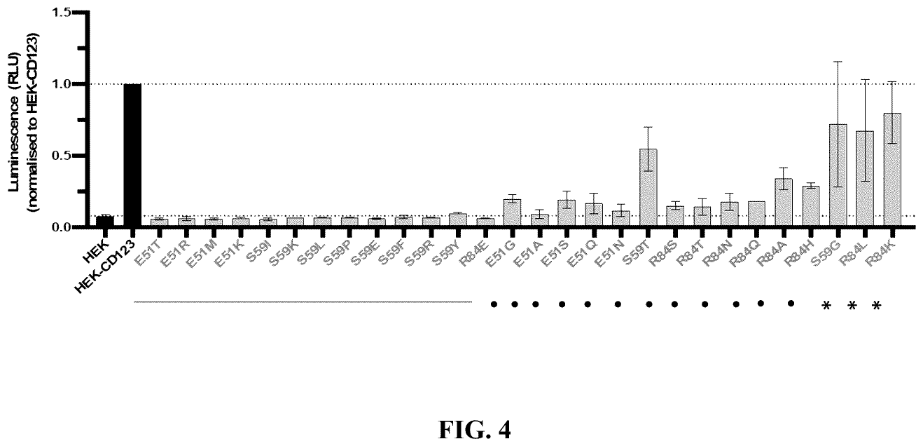

Relative luminescence signal measured after cocultivation of HEK, HEK-CD123 (wildtype) and HEK CD123 variant isoforms with Jurkat/FcγRIIIa/NFAT-Luc reporter cells. RLU is normalized to the signal measured with HEK-CD123 (wildtype) (FIG. 4).

### 7. T Cell Engager Assay: In Vitro Assay to Quantify T Cell Activation and Target Cell Killing by a CSL362-Derived T Cell Engager

### 7.1 Material and Methods

### Primary Human T Cell Isolation Culture

Leucocyte Buffy coats from anonymous healthy human donors were purchased from the blood donation center Basel (Blutspendezentrum SRK beider Basel, BSZ). Peripheral blood mononuclear cells (PBMCs) were isolated by density centrifugation using SepMate™ tubes (Stemcell technologies) with the density gradient medium Ficoll-Paque™ (GE Healthcare) according to the manufacturer's protocol. Human T cells were then purified (>96% purity) by magnetic negative selection using an EasySep Human T Cell Isolation Kit (Stemcell Technologies) according to the manufacturer's instruction. If frozen PBMCs were used, T cells were isolated after thawing and cultured in supplemented media without stimulation overnight. T cells were cultured in RPMI-1640 Medium (Sigma-Aldrich) supplemented with 10% heat-inactivated human serum (AB+, male; bought from BSZ Basel), 2 mM Glutamax, 10 mM HEPES, 1 mM Sodium Pyruvate, 0.05 mM 2-Mercaptoethanol and 1% MEM Non-essential amino acids (100×) (all Gibco Life Technologies).

### In Vitro BiTE-Mediated Killing Assay

For the BiTE killing assays the HEK-293 target cells were co-cultured together with primary human effector T cells and 300 ng/mL CD3/CSL365 BiTE at an effector to target ratio (E:T ratio) of 10:1 for 72 h.

Before initiation of the co-culture HEK, HEK-CD123 and the HEK stably expressing the CD123 variants were stained with CellTraceViolet (CTV) according to the manufacturer's protocol and cultured in 96-well culture plates in complete human medium overnight at 37° C., 5% CO2. The following day either thawed T cells, that were kept in supplemented medium overnight or freshly isolated T cells were added with the BiTE at a concentration of 300 ng/ml to the HEK-293 cells and kept for 72 h at 37° C. T-cell activation was assessed by quantifying the proportion of CD69% positive T-cells by flow cytometry. Cytotoxic activity and activation of T cells were analysed by flow-cytometry. Specific killing was calculated as follows: (1-Nr. alive target cells with BiTE/Nr. alive target cells without BiTE)*100. Cell morphology was assessed with the light microscope Axio Vert.A1 (Zeiss) at 20× magnification.

### 7.2 Results

### T Cell Engager-Mediated T Cell Activation

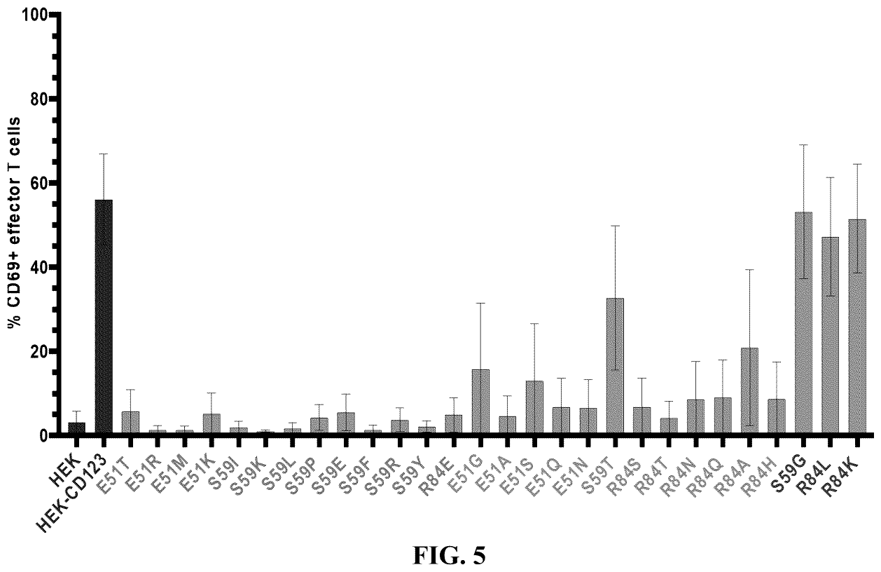

Stable target cell lines HEK, HEK-CD123 and CD123 variants were co-cultured with human primary pan T cells at an E:T ratio of 10:1 in the presence of 300 ng/mL CD3/CSL362 BiTE. FIG. 5 shows a summary of % CD69⁺ T cells measured after co-culture with HEK, HEK-CD123 or all CD123 variants. The data are normalized to % CD69⁺ cells in the presence of HEK target cells. Error bars show mean±SD. Data represent 5 independent blood donors and experiments with 2 technical replicates per group.

### T Cell Engager-Mediated Cell Killing

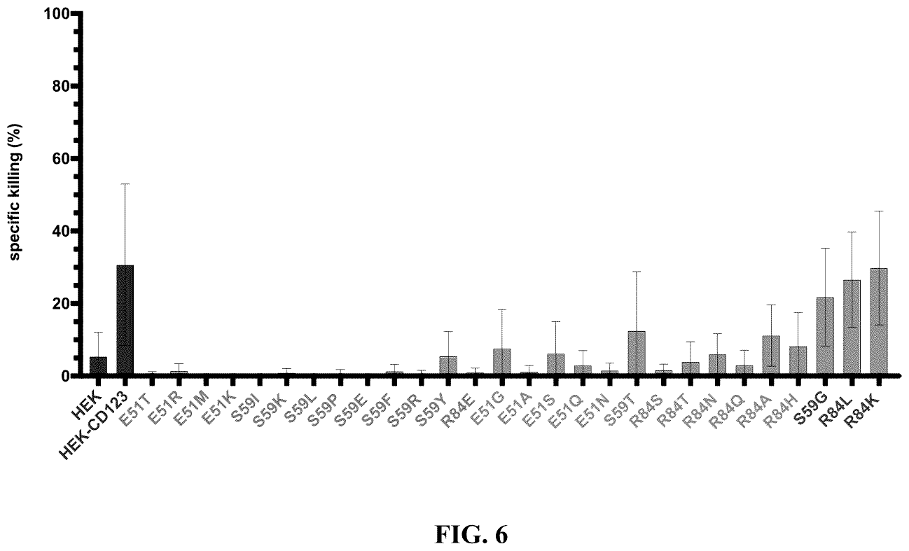

Stable target cell lines HEK, HEK-CD123 and CD123 variants were co-cultured with human primary pan T cells at an E:T ratio of 10:1 in the presence of 300 ng/mL CD3/CSL362 BiTE. FIG. 6 shows the specific BiTE-mediated killing of HEK, HEK-CD123 and all CD123 variants in % after 72 h co-culture. Error bars show mean±SD. Data represent 5 independent blood donors and experiments with 2 technical replicates per group.

### 8. CAR T Killing

### 8.1 Material and Methods

### Flow Cytometry and Cell Sorting

Flow cytometry was performed on BD LSRFortessa with the BD FACSDiva Software, and the data was analysed with FlowJo Software (FlowJo version 10.7.1).

The primary T cells were washed in ice-cold PBS followed by staining with a fixable viability dye for 20 min at 4° C. in the dark. Thereafter, cell surface staining was performed in 50 μl FACS Buffer (PBS+2% FCS+0.1% NaN3) with the fluorescently labelled antibodies for 20 min at room temperature in the dark. In the case of biotin-labelled antibodies, the secondary antibody with streptavidin was subsequently stained with the same protocol. The stained cells were washed once in FACS Buffer and then acquired immediately.

For cell sorting the cells were pelleted and resuspended in FACS Buffer (PBS+2% FCS) supplemented with 1 mM EDTA and kept on ice until analysis. FACS was performed either on BD FACSAria or BD FACSMelody Cell Sorter. The control cells were also subjected to the sorting process.

### Design and Production of the CD123-CAR HDRT

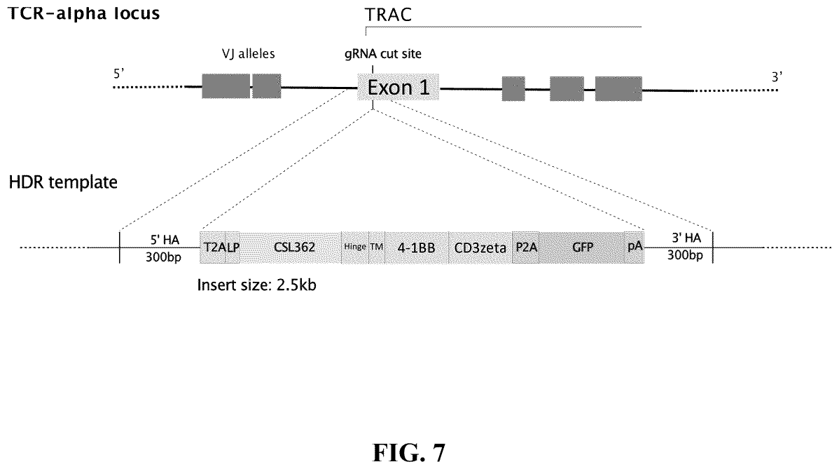

The CAR T cells were generated by co-electroporation of CRISPR-Cas9 Ribonucleoproteins (RNPs) specific for the TRAC locus (SEQ ID NO: 24) and a double-stranded DNA HDR template (HDRT). The HDRT encodes a second-generation CD123−specific CAR (SEQ ID NO: 15) with the single-chain variable fragment (scFv) of clone CSL362 (kind gift from D. Neri and published previously (HUTMACHER et al. 2019), the CD8alpha hinge (SEQ ID NO: 25) and transmembrane domain (SEQ ID NO: 26) (Gen CD8A ENSG00000153563), the intracellular signaling moieties 4-1BB (SEQ ID NO: 27) (Gen TNFRSF9 ENSG00000049249) and CD3zeta (SEQ ID NO: 28) (Gen CD247 ENSG00000198821), as well as the fluorescent reporter protein GFP. It is flanked by symmetric arms of homology (300 bp) complementary to the TRAC locus Exon 1 (FIG. 7). The construct was synthesized and cloned into a cloning vector (pUC57 backbone) by GenScript® and the HDRT was PCR-amplified from the plasmid using Kapa high-fidelity polymerase (Kapa Hifi Hotstart Ready Mix, Roche). The PCR amplicon was purified with NucleoSpin Gel and PCR clean-up kit (Macherey-Nagel) according to the manufacturer's instruction and the correct size was verified by gel electrophoresis on a 1% agarose gel. The HDRT was then condensed to a final concentration of 1 ug/μl using vacuum concentration, and stored at −20° C. until usage.

Genomic DNA Extraction and Sequencing from Human T Cells

Genomic DNA was extracted from genetically modified human T cells by resuspension in 100 μl Tail Lysis Buffer (100 mM Tris [pH 8.5], 5 mM Na-EDTA, 0.2% SDS, 200 mM NaCl) containing Proteinase K (0.1 μg; Sigma-Aldrich) and incubation at 56° C. on an Eppendorf Thermomix Comfort shaker at 1000 rpm. After 1 h the enzyme was heat-inactivated at 95° C. for 15 min. Following centrifugation (14000 rpm, 10 min), the DNA was precipitated by mixing the samples in a 1:1 volume ratio with isopropanol. The DNA was pelleted, washed in 70% ethanol, air dried and resuspended in Milli-Q water. Finally, the DNA concentration was measured with a NanoDrop™ device (Thermo Fisher). In order to verify the correct insertion of the CAR-transgene into the TRAC locus the primers were designed outside the 5′ and 3′ arms of homology. Following PCR with Phusion High-Fidelity DNA Polymerase (Thermo Scientific) the PCR products were size-separated by gel electrophoresis on a 0.8% agarose gel and the correct amplicon length was purified using the Nucleospin PCR and Gel Clean-up kit (Macherey-Nagel; according to the manufacturer's protocol). 100 ng of the eluted DNA was utilized for an additional PCR amplification with the same primers in order to increase the DNA quantity. The PCR amplicons were cleaned using the Nucleospin PCR and Gel Clean-up kit (Macherey-Nagel) and ligated into the pJET 1.2/blunt cloning vector for 2 h at 22° C. using the ConeJET PCR Cloning Kit (Thermo Scientific). Following bacterial transformation into competent Bacteria (JM109) and overnight incubation at 37° C., 32 colonies were picked and screened by PCR. The colonies with the correctly integrated transgene were inoculated in 5 ml LB-Medium supplemented with 50 ug/ml ampicillin and grown overnight at 37° C. Plasmid DNA was isolated from the cultured bacteria using the GenElute Plasmid Miniprep Kit (Sigma-Aldrich) following the manufacturer's protocol. Sanger Sequencing was performed at Microsynth AG Switzerland. Sequences were analysed using MegAlign Pro (DNASTAR, Version 17.0.1.183).

### Generation of Human CD123-CAR T Cells by Non-Viral CRISPR/Cas9-Based Editing

Cas9 Ribonucleoproteins (RNPs) were freshly generated prior to each electroporation. Thawed crRNA (specific for the TRAC locus and previously published ROTH et al. 2018) and tracrRNA (both purchased from IDT Technologies, resuspended at 200 μM) were mixed in a 1:1 molar ratio (120 pmol each), denatured at 95° C. for 5 min, and annealed at room temperature for 10 to 20 min in order to complex an 80 μM single guide RNA (sgRNA) solution. Poly-Glutamic Acid (PGA; 15-50 kDa at 100 mg/ml; Sigma-Aldrich) was added to the sgRNA in a 0.8:1 volume ratio. Lastly, to complex ribonucleoproteins (RNPs) 60 pmol recombinant Cas9 (University of California Berkeley at 40 μM) was mixed with the freshly prepared sgRNA (molar ratio Cas9:sgRNA=1:2) and incubated for 20 min at RT in the dark.

Prior to electroporation isolated human T cells were activated for 48 h with CD3/CD28 Dynabeads (Thermofisher) at a cell to beads ratio of 1:1 together with the recombinant human cytokines IL-2 (150 U/ml; Proleukin purchased at the University Hospital Basel), TL-7 (5 ng/ml; R&D Systems) and IL-15 (5 ng/ml; R&D systems) at 37° C. with 5% CO2 at a cell density of 1.5-2×10⁶ cells/ml.

Electroporation was performed with the 4D-Nucleofector™ system (Lonza) with Program EH-115. Following activation, the CD3/CD28-Dynabeads were removed by putting the resuspended T cells in an EasySep™ magnet for 2 min. For each electroporation 1×10⁶ activated T cells were used, washed once in PBS and then resuspended in 20 μl Lonza supplemented P3 electroporation buffer. In a separate 96-well culture plate the HDRT (3-4 ug) and RNPs (60 pmol) were thoroughly mixed and incubated for 5 min. The cells were then added, mixed and the whole volume was transferred into the 16-well Nucleocuvette™ Strips. Immediately following electroporation 80 μl of prewarmed supplemented medium was added to each cuvette and incubated at 37° C. After 20 min the cells were transferred into 48-well culture plates at 1×10⁶ cells/ml and replenished with IL-2 500 U/ml. The medium and IL-2 was replenished every 2 days, and the cells were kept at a cell density of 1×10⁶ cells/ml. Following flow-sorting at day 3-5 post-electroporation the supplemented medium was complemented with 1% Penicillin-Streptomycin (10′000 U/ml) and IL-2 (50 U/ml) and the cells were expanded for 5-6 days until used for the subsequent experiments. Control T cells were electroporated with an incomplete RNP (missing the specific crRNA), otherwise processed exactly identical to the CAR T cells.

### In Vitro Human CD123-CAR Killing Assay

The day before the co-culture HEK-293 target cells (HEK, HEK-CD123, CD-123 variants) were stained with CTV according to the manufacturer's instruction and kept in supplemented human Medium overnight at 37° C. and 5% CO2. The flow-sorted, expanded GFP+CAR T cells and control cells were added to the target cells in an effector to target ratio 10:1 and co-cultured for 24 h at 37° C., 5% CO2. Specific killing and T cell activation was measured by flow cytometry. Specific killing was calculated according to the indicated formula: (1-Nr. alive target cells in co-culture with CAR T cells/Nr. alive target cells in co-culture with control cells)*100. Using the microscope Axio Vert.A1 (Zeiss) cell morphology was recorded.

### Human Cytokine Measurement (ELISA)

The supernatant of the co-culture experiments (BiTE and CAR) was harvested and stored at −20° C. for human cytokine measurements. IFNγ was measured using the colorimetric ELISA MAX Standard Set Human IFNγ kit (BioLegend) according to the manufacturer's instruction. In short, the human IFNγ specific capture antibody was coated on a 96-well plate and incubated overnight at 4° C. The following day the samples (dilution 1:10) and standards were added and incubated with a biotinylated anti-human IFNγ detection antibody. Subsequently, Avidin-horseradisch peroxidase solution was added and color development was induced using the colorimetric substrate TMB, that was terminated with the stop solution. The optical density was read at 450 nm with the microplate reader A standard curve calculated from standard dilutions was run in duplicates with every experiment. The data is depicted in pg/ml.

### Statistical Analysis

Statistical Analysis was performed on Prism 9.1.2 software (GraphPad). N values are found within each figure legend.

### 8.2 Results

### Car T-Cell Mediated T Cell Activation

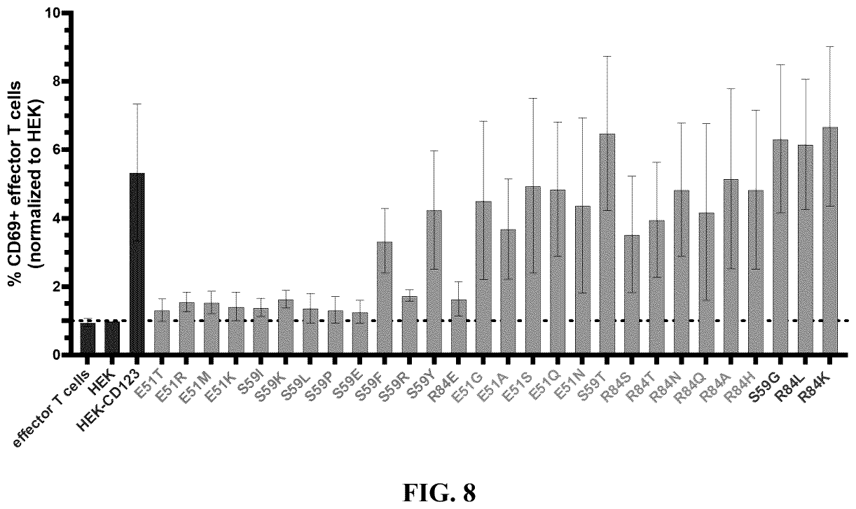

FIG. 8 shows FACS plots representing effector T cell activation (CD69) after 1 day co-culture of target cells HEK, HEK-CD123, E51K and either control cells or CD123−specific CAR T cells. Summary of CD69⁺ CAR T cells either alone (effector T cells) or in the presence of HEK, HEK-CD123 or all CD123 variants after 24 h co-culture. The data are normalized to % CD69⁺ cells in the presence of HEK target cells.

### Car T-Cell Mediated Cell Killing

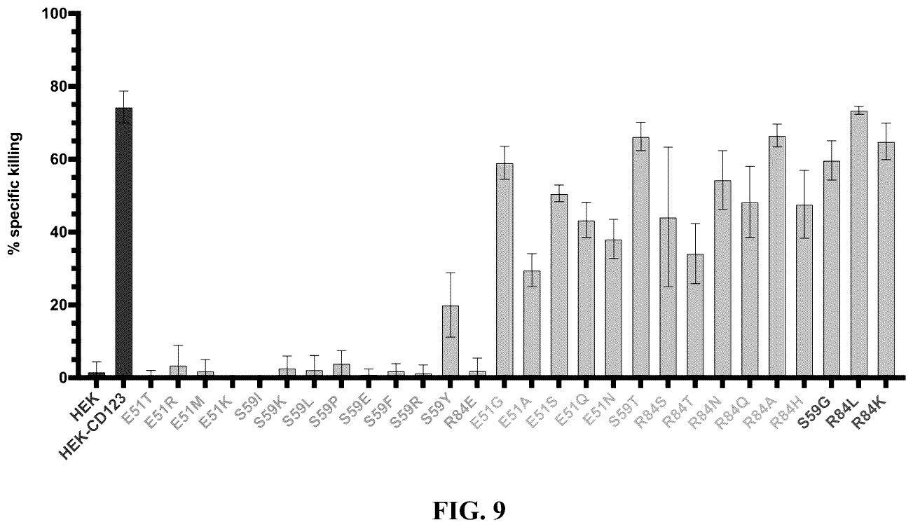

FIG. 9 shows the quantification of specific killing measured by flow cytometry of HEK, HEK-CD123 and its variants by CD123-specific CAR T cells at day 1 of co-culture. Error bars show mean±SD. Data from 3 independent blood donors and experiments with 2 technical replicates per group.

### 9. Affinity Measurements for Selected Variants

### 9.1 Material and Methods

All BLI experiments were performed on an Octet RED96e or Octet R8 at 25° C. with shaking at 1000 rpm using 1× Kinetic Buffer (Sartorius, PN: 18-1105).

CSL362 hIgG1 Binding to CD123 ECD Wt and Variants

Binding of antibody CSL362 hIgG1 to CD123 ECD wt. and variants (analytes) was performed at low (50 nM) and high (300 nM) concentration of analyte.

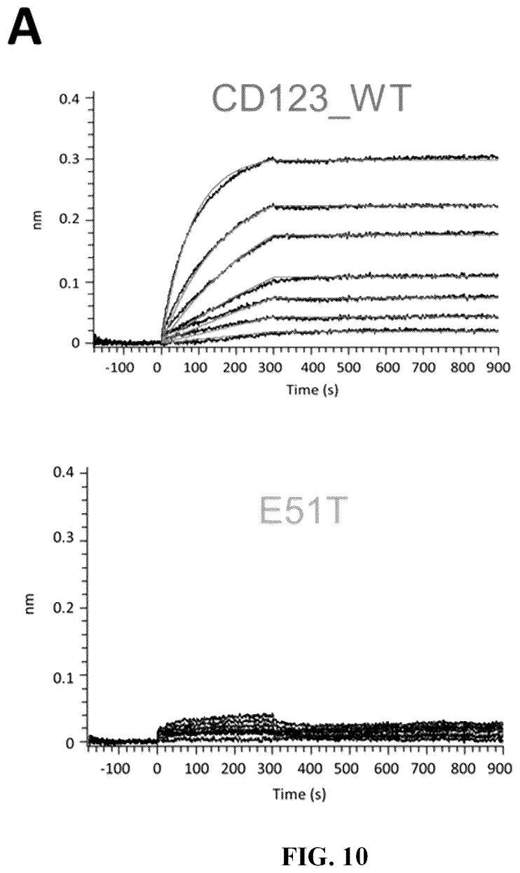

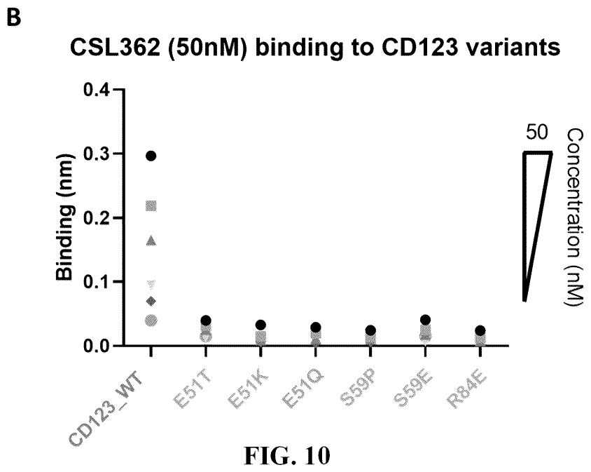

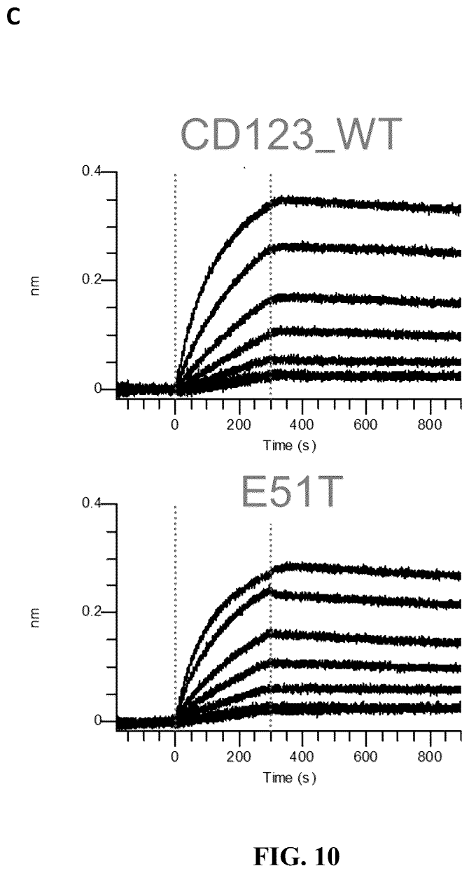

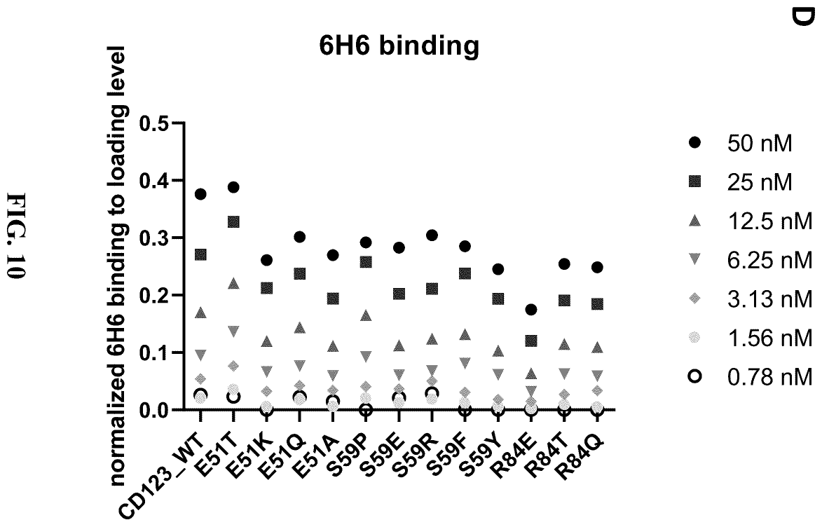

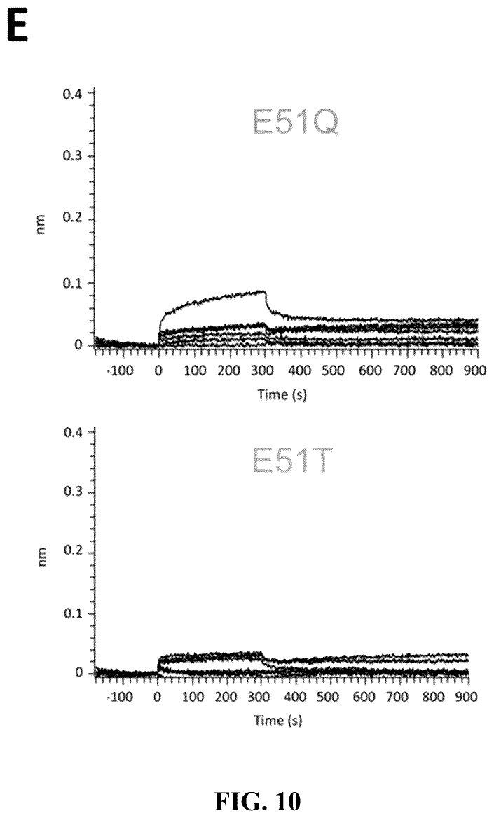

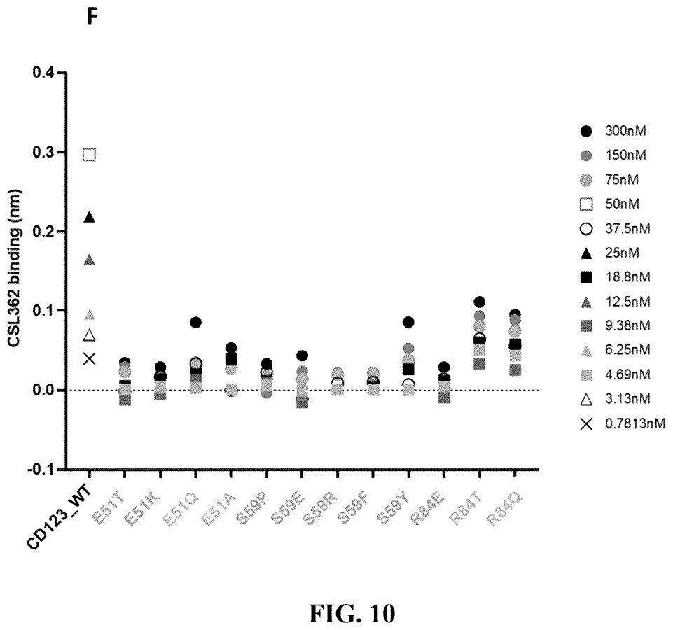

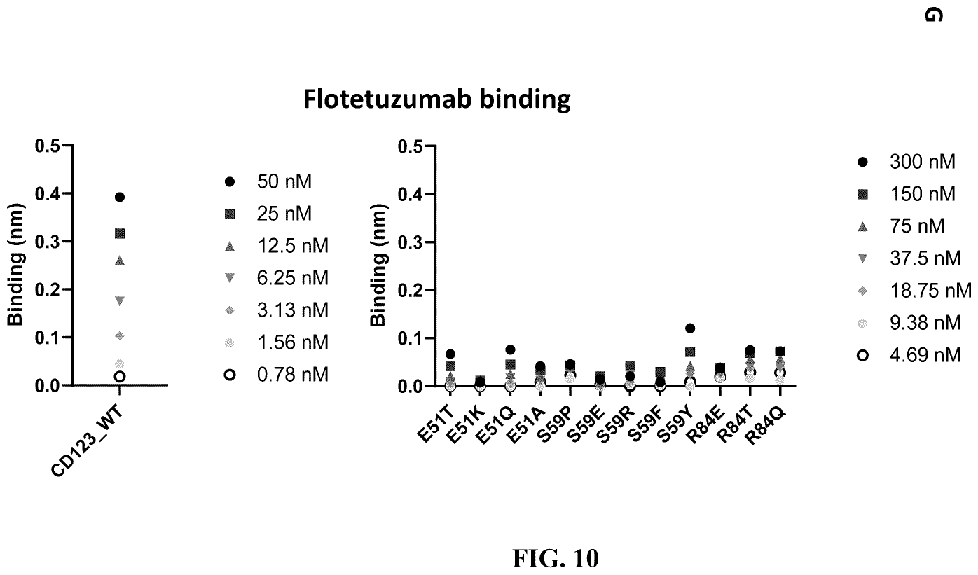

Antibody CSL362 hIgG1 was captured by Anti-Human Fc capture biosensor (AHC) (Sartorius, PN: 18-5060) for 300 s at 0.5 μg/mL. Analytes CD123 ECD wt. and variants (only CD123 ECD variants at high analyte concentrations) were titrated at 7 concentrations from 50 nM to 0.78 nM (300 nM to 4.7 nM). Association to analyte was monitored for 300 s and dissociation for 600 s (900 s). Double reference subtraction was performed against buffer only well and biosensor loaded with a negative hIgG1 control. Regeneration was performed in 10 mM Gly-HCl ph1.7. Data were analyzed using the Octet Data Analysis software HT 12.0. Data were fitted (when possible) to a 1:1 binding model. Kinetic rates ka and kd were globally fitted. In qualitative illustration in FIGS. 10A, B, E and F binding level were taken at 280 s association for all concentrations.

6H6 mTgG1 Binding to CD123 ECD Wt. And Variants

Binding of antibody 6H6 mIgG1 (Biolegend, PN: 306002) to CD123 ECD wt and variants (analytes) was performed using Streptavidin capture biosensor (SA) (Sartorius, PN: 18-5019). CaptureSelect™ Biotin Anti-LC-kappa (Murine) (Thermo Fischer, PN: 7103152100) was captured for 600 s at 1 μg/mL on SA tips. Those biosensors were then used to capture antibody 6H6 mIgG1 for 300 s at 2.5 μg/mL. Analytes CD123 ECD wt. and variants were titrated at 7 concentrations from 50 to 0.78 nM. Association to analyte was monitored for 300 s and dissociation for 600 s. Buffer only well was used as reference. Regeneration was performed in 10 mM Gly-HCl pH 1.7. Data were analyzed using the Octet Data Analysis software HT 12.0. Data were fitted (when possible) to a 1:1 binding model. Kinetic rates ka and kd were globally fitted. In qualitative illustration in FIGS. 10C and D binding level were taken at 250 s association for all concentrations.

Flotetuzumab hIgG1 Binding to CD123 ECD Wt and Variants

Binding of antibody flotetuzumab hIgG1 to CD123 ECD wt was performed at low (50 nM) concentration of analyte and to CD123 variants (analytes) high (300 nM) concentration of analyte.

Antibody flotetuzumab hIgG1 was captured by Anti-Human Fc capture biosensor (AHC) (Sartorius, PN: 18-5060) for 300 s at 0.5 μg/mL. Analytes CD123 ECD wt and variants (only CD123 ECD variants at high analyte concentrations) were titrated at 7 concentrations from 50 nM to 0.78 nM or 300 nM to 4.7 nM, respectively. Association to analyte was monitored for 300 s and dissociation for 600 s (CD123 wt) and 900 s (CD123 variants). Double reference subtraction was performed against buffer only well and biosensor loaded with a negative hIgG1 control. Regeneration was performed in 10 mM Gly-HCl pH 1.7. Data were analyzed using the Octet Data Analysis software HT

12.0. Data were fitted (when possible) to a 1:1 binding model. Kinetic rates ka and kd were globally fitted. In qualitative illustration in FIG. 10G binding level were taken at 250 s association for all concentrations.

### 9.2 Results

CSL362 antibody was immobilized and subsequently binding to recombinant CD123 wt or the indicated variants was measured at various concentrations as a function of time. While wildtype CD123 was bound in a dose-dependent manner (FIG. 10 A, B), the variants E51T, E51K, E51Q, S59P, S59E, and R84E did not bind CSL362 up to 50 nM (FIG. 10 B). Due to the absence of binding the inventors were unable to determine an affinity. In contrast, wildtype CD123 and all tested variants bound the control antibody 6H6 which binds an epitope that is not overlapping with any of the variants. The only variant with reduced binding was R84E which could indicate that R84E leads to reduced protein stability (FIG. 10 D). Since the inventors could not determine an affinity to CSL362 at 50 nM maximum analyte concentration, the experiment was repeated with up to 300 nM analyte. At these concentrations the wildtype CD123 could no longer be measured. Even at this very high protein concentration none of the tested variants characterized as “non-binder” (E51T, E51K, E51A, S59P, S59E, S59R; S59F; R84E) resulted in detectable binding (e.g. FIG. 10 E, F). Variants that resulted in very weak binding at 300 nM (E51Q, S59Y, R84T and R84Q) were characterized as “weak binder”. Thus, for all non-binders tested no interaction could be detected between the variants and CSL362 while binding to 6H6 was preserved, except for variant R84E.

### 10. CD123 Isoforms Preserve Interleukin-3 Binding

### 10.1 Material and Methods

hIL3 Binding to CD123 ECD Wt. And Variants

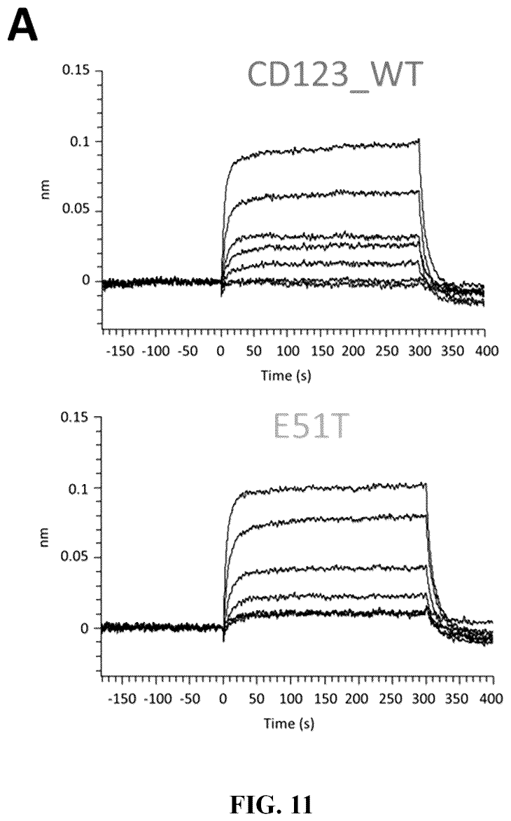

Binding of hIL3 (SinoBiological, PN: 11858-H08H) (analyte) to CD123 ECD wt. and variants (ligands) was performed using Streptavidin capture biosensor (SA) (Sartorius, PN: 18-5019). CD123 ECD wt. and variants were biotinylated using Biotinylation kit Type B (Abeam, PN: ab201796) following manufacturer instructions. Biotinylated CD123 ECD wt. and variants (ligands) were captured on SA tips for 1000 s to achieve a nm shift of 1.5 to 2 nm. Analyte hIL3 was titrated at 7 concentrations from 500 to 7.8 nM. Association to analyte was monitored for 300 s and dissociation for 120 s. Buffer only well was used as reference. No regeneration was performed, and a new set of tips was used for each biotinylated captured ligand. Data were analyzed using the Octet Data Analysis software HT 12.0. Due to the fast on/off nature of the interaction data were analyzed using Steady state analysis. Qualitative representation in FIG. 11 binding level were taken at 250 s association for all concentrations.

### 10.2 Results

Recombinant wildtype CD123 or the indicated variants were biotinylated and captured. Interleukin-3 (IL-3) binding was determined as a function of time.

Recombinant wildtype CD123 and the indicated variants bound IL-3 in a dose-dependent manner. Thus, the non-binding variants do not bind CSL362 but the protein is normally bound by a control antibody 6H6 and IL-3 (FIG. 11).

The variants S59P and R84E show a slightly decreased binding to IL3 compared to the WT.

### 11. Thermal Unfolding

### 11.1 Material and Methods

DSF analysis were performed on a Bio-Rad CFX96 Touch Deep Well RT PCR Detection System. Sypro Orange 5000X in DMSO (Sigma, PN: S5692) was used at a final concentration of 5×. Temperature gradient from 25 to 95° C. in increment of 1.5° C. in a reaction volume of 20 uL. “FRET” scan mode was used to monitor fluorescence. All samples were analyzed at a final concentration of 0.25 mg/mL in triplicate. The temperature of protein unfolding transition Tm was calculated using the 1st derivative method.

### 11.2 Results

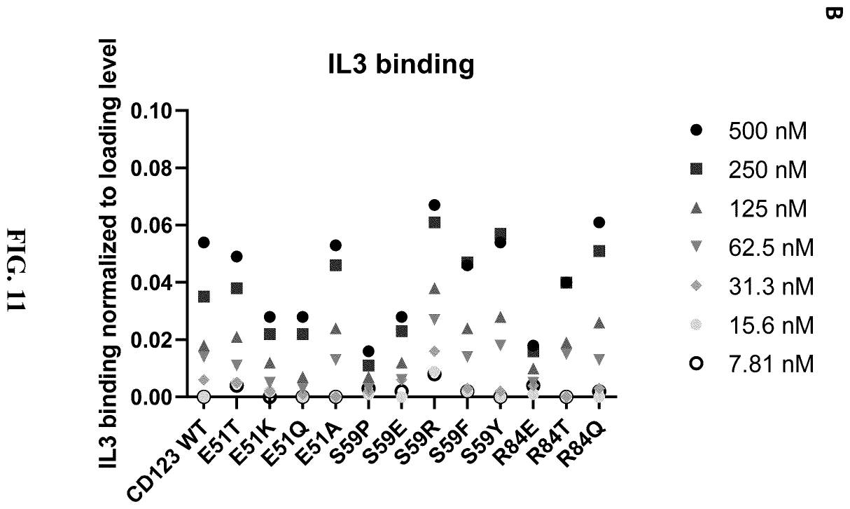

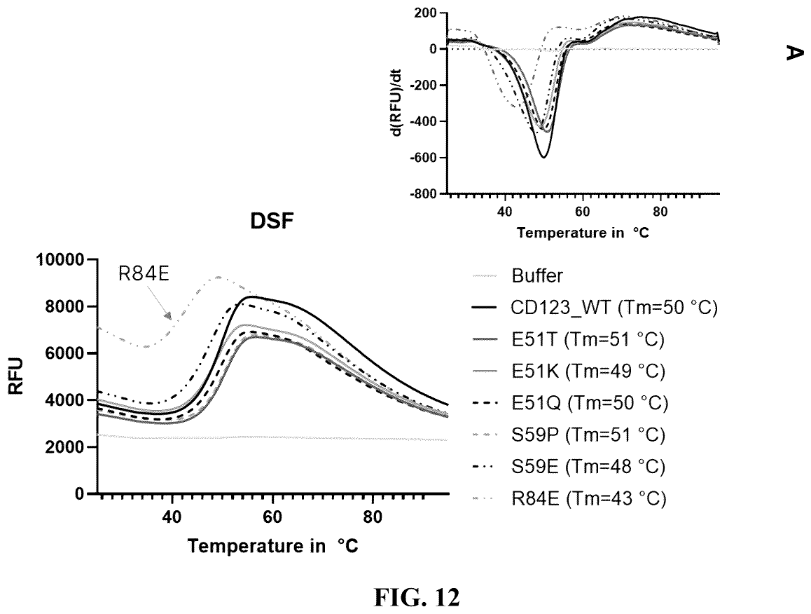

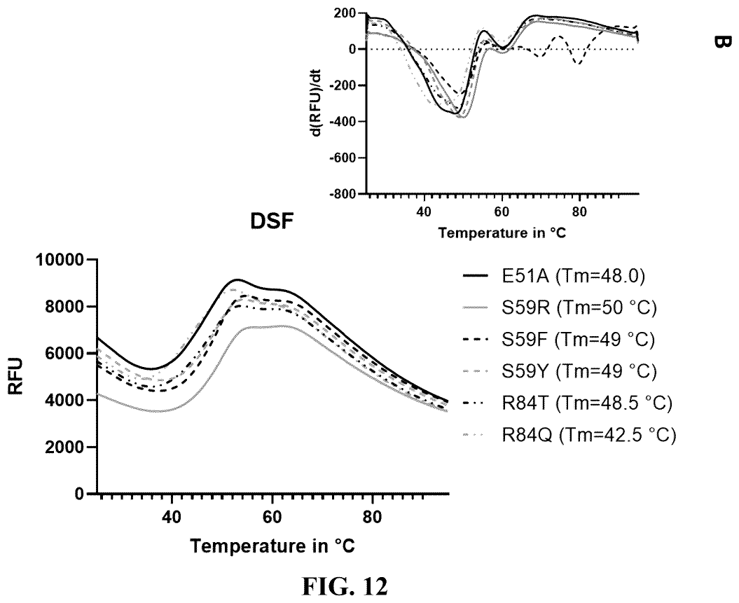

Thermal unfolding measurements demonstrated that most variants except had a thermostability comparable to wildtype CD123 (FIG. 12). R84E had reduced thermostability. This result might explain why R84E demonstrated reduced binding to the control antibody 6H6 that binds an epitope not affected by the R84E mutation itself. In addition, variant R84Q has a lower Tm.

### 12. Genetically Engineered TF-1 Cells Expressing CD123 Variants

### 12.1 Material and Methods

The erythroleukemic human cell line TF-1 (DSMZ No. ACC 334) was used as a model for hematopoietic stem cells (HSCs). TF-1 cells were electroporated with CRISPR-Cas9 and HDR template in order to knock-in E51K and E51T variants into endogenous DNA of CD123.

Cas9 Ribonucleoproteins (RNPs) were freshly generated prior to each electroporation. Thawed crRNA and tracrRNA (both purchased from IDT Technologies, resuspended at 200 μM) were mixed in a 1:1 molar ratio (120 pmol each), denatured at 95° C. for 5 min, and annealed at room temperature for 10 to 20 min in order to complex an 80 μM single guide RNA (sgRNA) solution. Poly-Glutamic Acid (PGA; 15-50 kDa at 100 mg/ml; Sigma-Aldrich) was added to the sgRNA in a 0.8:1 volume ratio. Lastly, to complex ribonucleoproteins (RNPs) 60 pmol recombinant Cas9 (University of California Berkeley at 40 μM) was mixed with the freshly prepared sgRNA (molar ratio Cas9:sgRNA=1:2) and incubated for 20 min at RT in the dark. Five minutes before electroporation, 50 pmols of the HDR templates were added to the complexed RNP (60 pmols).

Electroporation was performed using the Neon™ transfection system (Thermo Fischer). For each electroporation 0.2×10⁶ TF-1 cells were used, washed twice in PBS and then resuspended in 10 μl R buffer. Cells in R buffer were mixed with the complex RNP/HDR template and electroporated with the conditions 1200V, 40 ms, 1 pulse. After electroporation the cells were transferred into 48-well culture plates at 0.4×10⁶ cells/ml in fresh medium with GM-CSF (RPMI-1640, 10% FCS, 1% Glutamax, 2 ng/ml GM-CSF) and divided every 2 days.

Twelve days after electroporation engineered cells were bulk sorted by flow cytometry according to their CD123 variants expression. Cells were stained with MIRG123− biotin/Strep-PE and 6H6-Bv650. E51K and E51T knock-in (MIRG123−, 6H6⁺), CD123 knock-out (MIRG123−, 6H6−) and WT cells (MIRG123⁺, 6H6⁺) were sorted and cultured for 14 days before testing the functionality of the variants.

sgRNA used for CD123 KO:
(SEQ ID NO: 29)
GTCTTTAACACACTCGATAT

E51K HDR template:

(SEQ ID NO: 30)
TTTTAGATCCAAACCCACCAATCACGAACCTAAGGATGAAAGCAAAGGC
TCAGCAGTTGACCTGGGACCTTAACAGAAATGTGACaGAcATtaagTGT
GTTAAAGACGCCGACTATTCTATGCCGGTAAATCATACTCTCTATTGTT
TTTTTATTTT TATTTTATTTATTTATGTATTTA

E51T HDR template:

(SEQ ID NO: 31)
TTTTAGATCCAAACCCACCAATCACGAACCTAAGGATGAAAGCAAAGGC
TCAGCAGTTGACCTGGGACCTTAACAGAAATGTGACaGAcATtaccTGT
GTTAAAGACGCCGACTATTCTATGCCGGTAAATCATACTCTCTATTGTT
TTTTTATTTT TATTTTATTTATTTATGTATTTA

### 12.2 Results

TF-1 cells with a E51K and a E51T knock-in could be successfully generated. Cells were sorted and it could be confirmed that the E51K and E51T variant show a loss of binding to antibody MIRG123 (data not shown). Cells are sorted and analyzed for subsequent experiments.

### 13. Non-Binding Variants can be Enriched by Culture with IL3

### 13.1 Material and Methods

Twelve days after electroporation cells were washed twice in PBS before they were re-suspended at a concentration of 0.3 million cells/ml in 1 ml of medium with 2 ng/ml GM-CSF (RPMI 1640, 10% FCS, 1% Glutamax) or 10 ng/ml hIL3 (RPMI 1640, 10% FCS, 1% Glutamax). Cells were cultured for 6 days and were analysed by Flow Cytometry at days 0, 2, 4 and 6. The day of the analysis 200 μl of cells were taken from the culture and washed in PBS. They were then re-suspended in 200 μl of medium with 2 ng/ml GM-CSF in 96-well plate and incubated for 7 h at 37° C. and 5% CO2 before Flow Cytometry staining with MIRG123− biot/Strep-PE and 6H6-Bv650. Genomic DNA was extracted at days 0, 2, 4 and 6 from 200 μl of culture. On days 2, 4 and 6 400 μl of medium was added to the culture.

### 13.2 Results

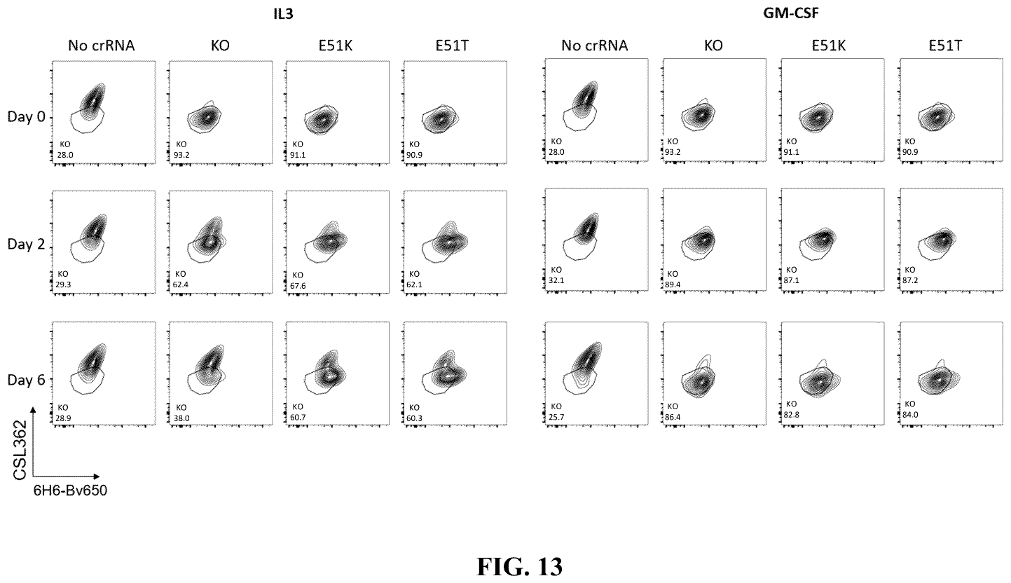

Bulk TF-1 cells were cultured as control (no crRNA), as KO (crRNA but no HDRT) or KI (crRNA plus KI HDRT, E51K or E51T). Control cells cultured with IL-3 or GM-CSF remained MIRG123+, 6H6+. KO cells cultured with GM-CSF largely remained MIRG123−, 6H6+. In contrast, in cultures containing KO cells cultured with IL-3 gradually, a MIRG123+, 6H6+ cell population became detectable. On day 6 the MIRG123+, 6H6+population dominated, demonstrating that cells expressing the CD123 receptor had a competitive advantage in presence of IL3. In KI cells (E51K or E51T) the population of MIRG123− 6H6+ but also a population of MIRG123+ 6H6+ cells gradually increased with IL3. This was much less pronounced in cells cultured with GM-CSF. Thus, KI cells (MIRG123− 6H6+) have a functional receptor. See FIG. 13.

### 14. Gene Edited Cells Remain Responsive to Stimulation with IL3

### 14.1 Material and Methods

TF-1 cell are known to be responsive to stimulation by GM-CSF, IL3, and SCF. TF-1 cells (wild type, knock-out, as well as E51K and a E51T knock-in cells were tested for their capacity of being stimulated with IL3. TF-1 (ACC 334, DSMZ) cells were maintained in RPMI-1640 media supplemented with 10% heat-inactivated FCS, 2 mM GlutaMAX™ (Gibco) and 2 ng/ml hGM-CSF (215-GM, biot-techne). For Cas9 hybridisation with tracRNA/crRNA mix (SEQ ID No.s 29-31) and PGA were added (see the method described in Example 8.1 “_Generation of human CD123-CAR T cells by non-viral CRISPR/Cas9-based editing”). Just before electroporation, 50 pmols of 180 bp length ssDNA HDR template (Ultramer DNA Oligonucleotides, IDT) were added to the RNPs. For each electroporation, 200,000 TF-1 cells were washed two times with PBS and re-suspended in 10p R buffer (Neon™ Transfection System 10 uL). The mix of RNPs/HDR template was gently added and the cells were electroporated using the Neon Transfection System/Thermo Fisher) with the settings 1200V, 40 ms, 1 pulse. Electroporated cells were expanded every 2 to 3 days for 11 days for enrichment assays, and for 12 days before flow cytometry sorting for functional assays.

Sorted cells were washed once in PBS before they were distributed in 96-well white microplate clear flat bottom (Greiner bio-one). In each well 10,800 cells were cultured with increasing concentrations of hIL3 (0.2 ng/ml, 0.8 ng/ml, 3.13 ng/ml, 12.5 ng/ml, 50 ng/ml) in 150 μl of medium without GM-CSF (RPMI-1640, 10% FCS, 1% Glutamax). Cell proliferation was measured after 72 h at 37 C 5% CO2 using 15 μl of CellTiter-Glo® (Promega). Luminescence was read using the Synergy H1(BioTek) with integration time of Is.

### 14.2 Results

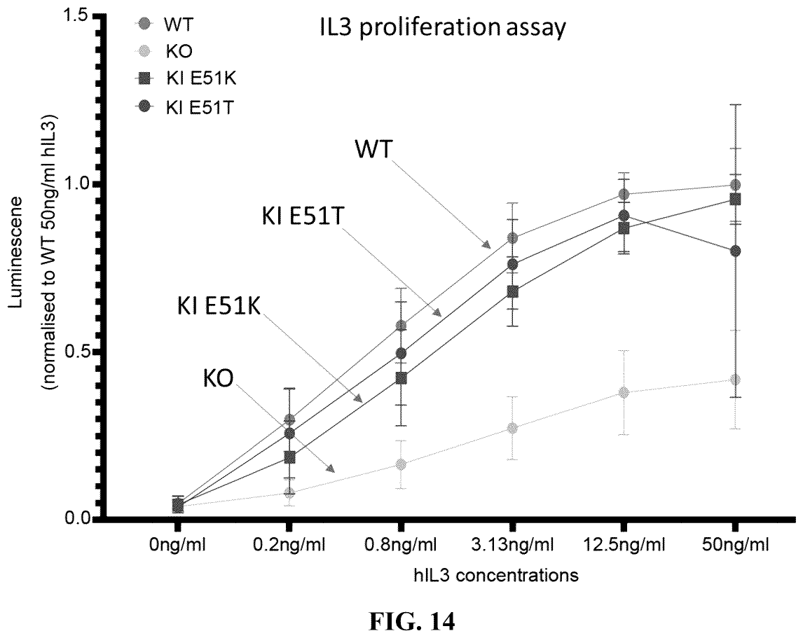

Results are shown in FIG. 14. TF-1 knock-in cells expressing the E51K and E51T variants of CD123 can proliferate upon add of hIL3 to a similar degree as TF-1 cells expressing wild-type CD123. Knock-out cells only show a minimal response to hIL3.

### 15. Gene Edited Cells are Protected from Blocking with MIRG123

### 15.1 Material and Methods

Antibody MIRG123 was tested for its capability to bind to TF-1 cell (wild type, knock-out, as well as E51K and a E51T knock-in cells).

Sorted cells were washed once in PBS before they were distributed in 96-well white microplate clear flat bottom (Greiner bio-one). In each well 10,800 cells were cultured with a fixed concentration of hIL3 (2.5 ng/ml) but different concentrations of MIRG123 (0.0013 nM, 0.004 nM, 0.012 nM, 0.036 nM, 0.11 nM, 0.33 nM, 1 nM) in 150 μl of medium
without GM-CSF (RPMI-1640, 10% FCS, 1% Glutamax). Cell proliferation was measured after 72 h at 37 C 5% CO2 using 15 μl of CellTiter-Glo® (Promega). Luminescence was read using the Synergy H1(BioTek) with integration time of Is.

### 15.2 Results

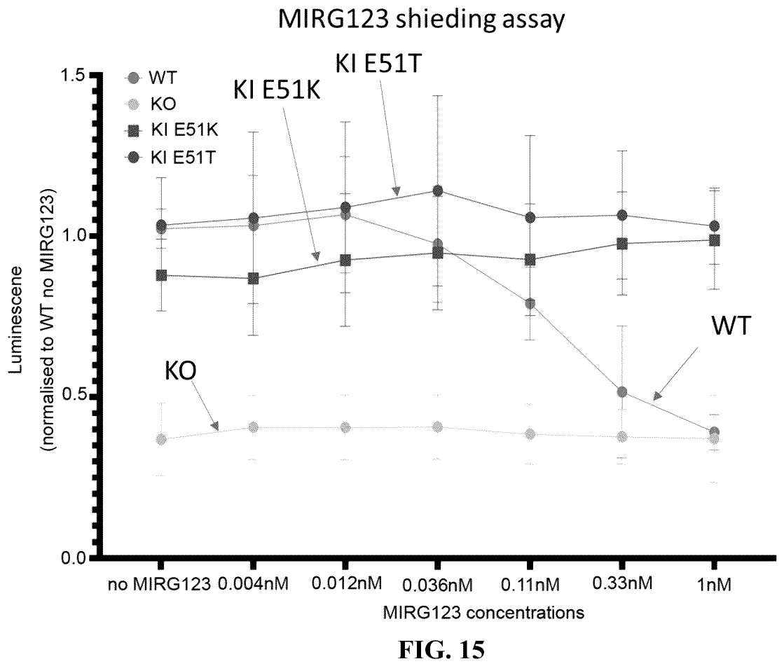

Results are shown in FIG. 15. MIRG123 leads to a dose-dependent proliferation blocking and apoptosis of IL3-stimulated wild type TF-1 cells. In contrast, TF-1 knock-in cells expressing the E51K and E51T variants of CD123 are efficiently protected from the blocking effects of MIRG123.

### 16. Editing of HSPCs

### 16.1 Material and methods

HSPCs were thawed in HSC-Brew GMP Basal Medium (Miltenyi) supplemented with HSC-Brew GMP Supplement, 2% human serum albumin, 100 ng/mL SCF, 100 ng/mL TPO, 100 ng/mL Flt3L and 60 ng/mL IL3 (Miltenyi) at a density of 0.5×10⁶ cells/ml. Cells were electroporated 2 days later. Thawed crRNA and tracrRNA (both purchased from IDT Technologies, resuspended at 200 μM) were mixed in a 1:1 molar ratio (120 pmol each), denatured at 95° C. for 5 min, and annealed at room temperature (RT) for 5 min in order to complex an 80P M guide RNA (gRNA) solution. Lastly, to complex ribonucleoproteins (RNPs) 1 μM Spyfi Cas9 (Aldevron at 61.889 μM) was mixed with the freshly prepared gRNA (molar ratio Cas9:gRNA=1:2) and incubated for 20 min at RT. During the RNP complexing, HSPC cells were collected, washed twice with electroporation buffer (Miltenyi) and resuspend in electroporation buffer at 1×10⁶ cells/50 μl. Cells were then mixed with the RNP (5 μl) and the HDRT (5 μl corresponding to 500 pmol) and the whole volume was transferred into the electroporation nucleocuvette. Electroporation was performed with a Miltenyi CliniMACS Prodigy using as settings 600V 100 s burst/400V 750 s square. Immediately after electroporation the cells were transferred to a 6 well plate and rested for 20 minutes at RT. After 20 min, 2 mL of pre-warmed HSPC medium supplemented with 100 ng/mL SCF, 100 ng/mL TPO and 100 ng/mL Flt3L was added into each well and the plate was incubated at 37° C. At different time points cells were collected and stained for Flow Cytometry with the antibodies 6H6-BV650 and MIRG123− biot/Strep-PE. Genomic DNA (gDNA) was extracted using DNA Quick extract (Lucigen) for sequencing analysis. The sgRNA used for the CD123 KO and the HDR templates for E51K and E51T are shown in Example 12 as SEQ ID NO:'s 29-31, respectively.

### 16.2 Results

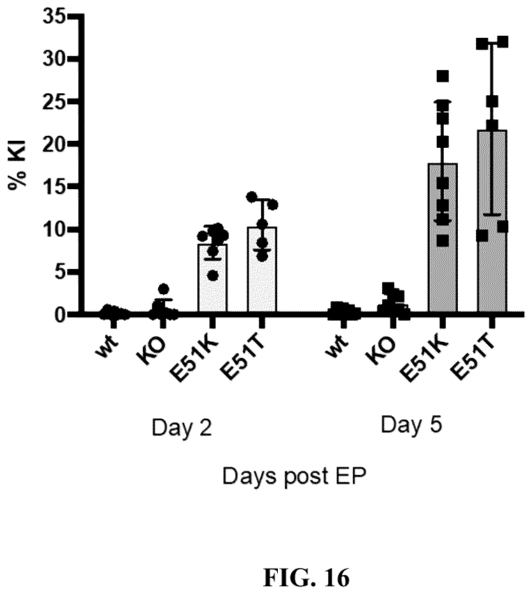

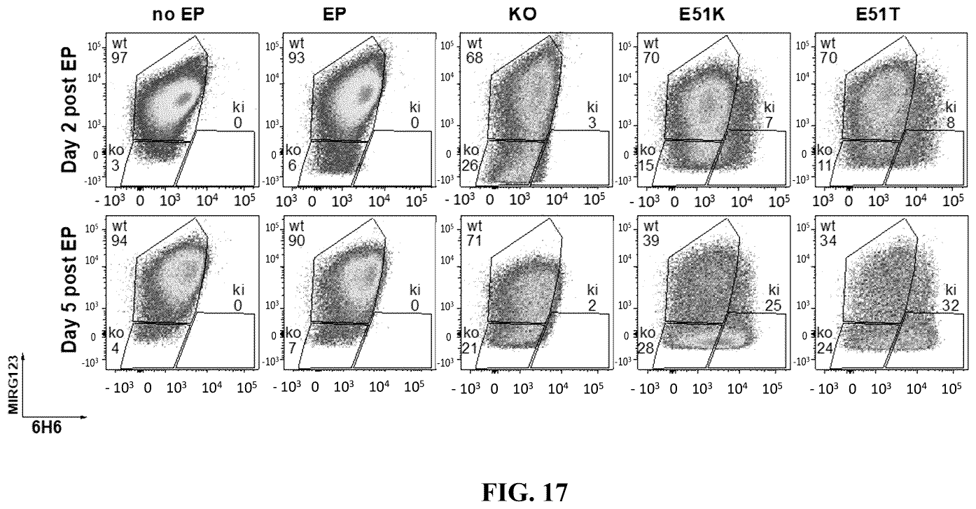

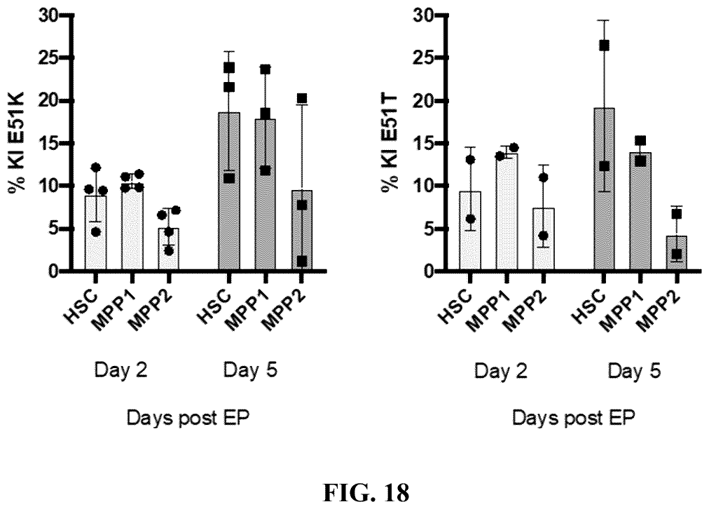

Mobilized, peripheral blood CD34+ HSPCs harboring a E51K and a E51T knock-in could be successfully generated (FIG. 16). Cells were analyzed by FACS and successful knock-in was validated by Sanger sequencing. HSPCs electroporated with RNP alone (KO) showed an increased fraction of CD123 negative cells. In contrast, E51K and E51T variants showed a loss of binding to antibody MIRG123 but preserved CD123 expression as assessed by the control antibody 6H6 (FIG. 17). Using CD90 and CD45 RA, we showed that also LT-HSCs (long term repopulating hematopoietic stem cells), as well as MPP1 cells (CD34⁺ CD38-CD90− CD45RA−) and MPP2 cells (CD34⁺ CD38− CD90− CD45RA+) were edited (FIG. 18).

### 17. In Vitro HSPC Killing Assay with BiTE

### 17.1 Material and Methods

For the BiTE killing assays the HSPCs were edited as described above. Human T cells of the same HSPC donor were isolated from PBMCs by magnetic negative selection using an EasySep Human T Cell Isolation Kit (Stemcell Technologies) according to the manufacturer's instruction. The isolated T cells were cultured in supplemented media without stimulation overnight. Two days after electroporation edited HSPCs were co-cultured in a 96 U-bottom plate together with human effector T cells at an effector to target ratio of 3:1 and the CD3/CSL362 BiTE at 100 ng/ml for 72 h at 37° C. Cytotoxic activity (specific killing and elimination of HSPCs) were analysed by flow-cytometry. Specific killing was calculated as follows: (number of alive target cells with BiTE/number of alive target cells without BiTE)*100.

### 17.2 Results

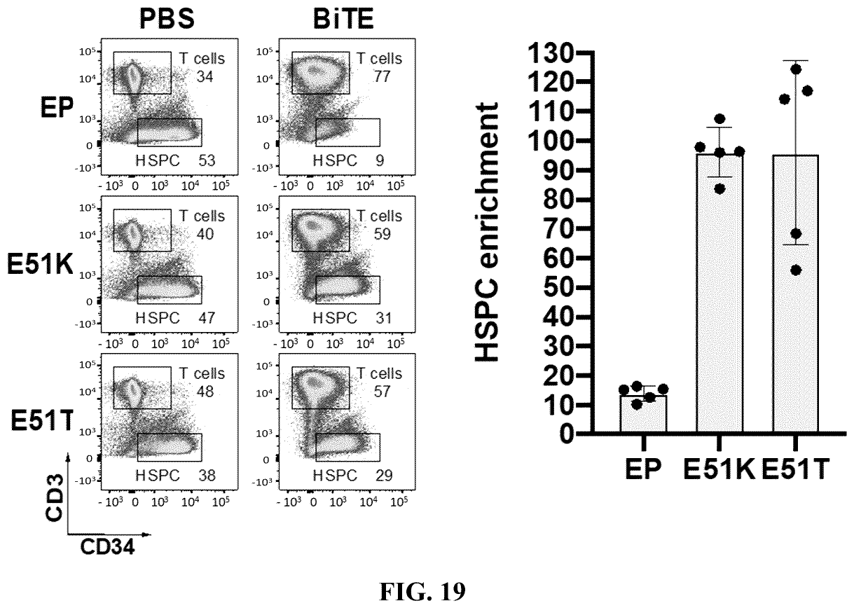

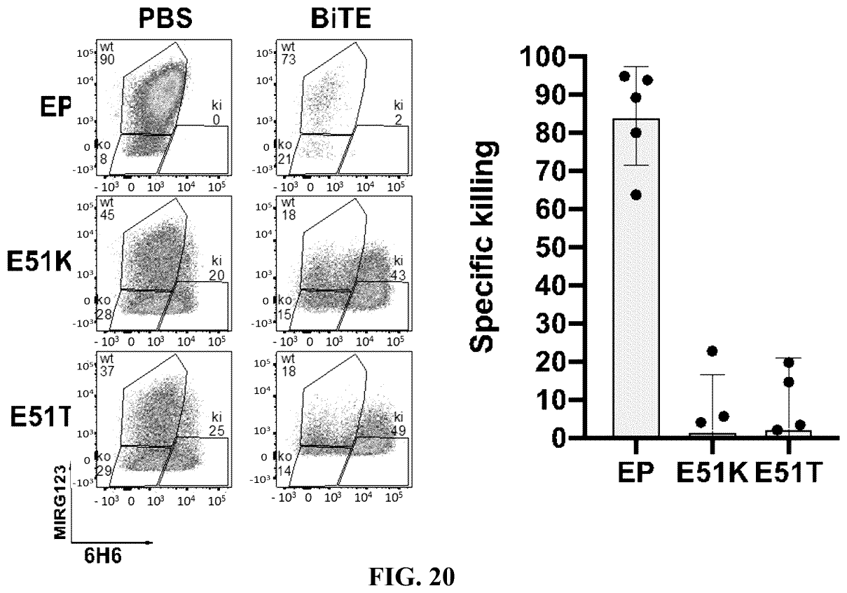

Co-culture of human effector T cells with control or edited HSPC cells (E:T=3:1) in the presence of the CD3/CSL362 BiTE at a concentration of 100 ng/ml led to a reduction of wild-type HSPCs upon treatment with the BiTE, as measured by quantification of the flow cytometry plots. In contrast, HSCs expressing the CD123 E51K or E51T variants were protected and enriched as evidenced by an increased cell population of MIRG123− 6H6+ cells. See FIGS. 19 and 20. In addition, after 72 hours of co-culture HSC cells were sorted using flow cytometry into CD123 antibody clone 6H6+ and 6H6− cells.

Enrichment of knock-in cells was confirmed by Sanger sequencing of the respective cell populations.

### 18. In Vivo Engraftment of Edited HSPCs

### 18.1 Material and Methods

HSC cells were edited as described above. One day after electroporation cells were collected, washed once with PBS and resuspended in PBS at a concentration of 10×10⁶ live cells/ml. Cells were injected i.v. in NSG-SGM3 female mice (3 weeks old) irradiated the day before with 200 cGy. Engraftment was monitored by FACS by analysing peripheral blood after 6 and 10 weeks staining for mouse and human CD45. Mice were euthanized 13 weeks after humanization for analysis of blood, spleen and bone marrow.

### 18.2 Results

Engraftment of HSPCs was quantified by measuring the percentage of human CD45 (human chimerism) in the spleen of mice 13 weeks after HSPC injection. Mice that received E51K or E51T knock-in HSPCs successfully engrafted and showed evidence of development of human CD45+ immune cells. In addition, the presence of B cells (measured by CD19), T cells (measured by CD3) and CD33+ myeloid cells among the human CD45 cells in the spleen and bone marrow demonstrate successful multilineage differentiation potential of engineered E51K or E51T knock-in HSPCs. Human HSPCs were detected in bone marrow.

### Sequences for Use in Practicing the Invention

Human CD 123 (SEQ ID NO: 1)
MVLLWLTLLLIALPCLLQTKEDPNPPITNLRMKAKAQQLTWDLNRNVTDIEC
VKDADYSMPAVNNSYCQFGAISLCEVTNYTVRVANPPFSTWILFPENSGKP
WAGAENLTCWIHDVDFLSCSWAVGPGAPADVQYDLYLNVANRRQQYECLH
YKTDAQGTRIGCRFDDISRLSSGSQSSHILVRGRSAAFGIPCTDKFVVFSQIEIL
TPPNMTAKCNKTHSFMHWKMRSHFNRKFRYELQIQKRMQPVITEQVRDRTS
FQLLNPGTYTVQIRARERVYEFLSAWSTPQRFECDQEEGANTRAWRTSLLIAL
GTLLALVCVFVICRRYLVMQRLFPRIPHMKDPIGDSFQNDKLVVWEAGKAG
LEECLVTEVQV VQKT
Anti-CD123 antibody (Flotetuzumab) VH (SEQ ID NO: 11)
EVQLVQSGAELKKPGASVKVSCKASGYTFTDYYMKWVRQAPGQGLEWIGD
IIPSNGATFYNQKFKGRVTITVDKSTSTAYMELSSLRSEDTAVYYCARSHLLR
ASWFAYWGQGTLVTVSS
Anti-CD123 antibody (Flotetuzumab) VL (SEQ ID NO: 12)
DFVMTQSPDSLAVSLGERVTMSCKSSQSLLNSGNQKNYLTWYQQKPGQPPK
LLIYWASTRESGVPDRFSGSGSGTDFTLTISSLQAEDVAVYYCQNDYSYPYTF
GQGTKLEIK
Anti-CD123 antibody (Vibecotabam) VH CDR1 (SEQ ID NO: 13)
QVQLQQSGAEVKKPGASVKVSCKASGYTFTDYYMKWVKQSHGKSLEW
MGDIIPSNGATFYNQKFKGKATLTVDRSTSTAYMELSSLRSEDTAVYYCA
RSHLLRASWFAYWGQGTLVTVSS
Anti-CD123 antibody (Vibecotabam) VL CDR1 (SEQ ID NO: 14)
DFVMTQSPDSLAVSLGERATINCKSSQSLLNTGNQKNYLTWYQQKPGQPP
KLLIYWASTRESGVPDRFTGSGSGTDFTLTISSLQAEDVAVYYCQNDYSYP
YTFGGGTKLEIK
Anti-CD123 CAR (SEQ ID NO: 15)
MALPVTALLLPLALLLHAARPDIVMTQSPDSLAVSLGERATINCESSQSLLNS
GNQKNYLTWYQQKPGQPPKPLIYWASTRESGVPDRFSGSGSGTDFTLTISSLQ
AEDVAVYYCQNDYSYPYTFGQGTKLEIKGGGGSGGGGSGGGGSEVQLVQSG
AEVKKPGESLKISCKGSGYSFTDYYMKWARQMPGKGLEWMGDIIPSNGATF
YNQKFKGQVTISADKSISTTYLQWSSLKASDTAMYYCARSHLLRASWFAYW
GQGTMVTVSSTTTPAPRPPTPAPTIASQPLSLRPEACRPAAGGAVHTRGLDFA
CDIYIWAPLAGTCGVLLLSLVITLYCKRGRKKLLYIFKQPFMRPVQTTQEEDG
CSCRFPEEEEGGCELRVKFSRSADAPAYQQGQNQLYNELNLGRREEYDVLD
KRRGRDPEMGGKPQRRKNPQEGLYNELQKDKMAEAYSEIGMKGERRRGKG
HDGLYQGLSTATKDTYDALHMQALPPR
gRNA TRAC (SEQ ID NO: 24)
AGAGTCTCTCAGCTGGTACA
CD8-Hinge domain (SEQ ID NO: 25)
TTTPAPRPPTPAPTIASQPLSLRPEACRPAAGGAVHTRGLDFACD
CD8-transmembrane domain (SEQ ID NO: 26)
IYIWAPLAGTCGVLLLSLVIT
4-1BB cytoplasmic domain (SEQ ID NO: 27)
KRGRKKLLYIFKQPFMRPVQTTQEEDGCSCRFPEEEEGGCEL
CD3-zeta domain (SEQ ID NO: 28)
RVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTAT
KDTYDALHMQALPPR

1. A mammalian cell or a population of cells expressing a first isoform of a CD123 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein, and wherein said first and second isoform are functional.

2. The mammalian cell or population of cells for use according to claim 1 wherein said first and said second isoform of CD123 are functional with respect to IL-3 binding, IL-3 dependent proliferation, expression on the cell surface or intracellular signaling capacity.

3. The mammalian cell or population of cells for use according to claim 1, wherein said polymorphic or genetically engineered allele is characterized by at least one substitution of an amino acid in position E51 or S59 of SEQ ID NO: 1.

4. The mammalian cell or population of cells for use according to claim 3 wherein said residue E51 is substituted by an amino acid selected from the group consisting of: K, N, T, R, M, G and A, and/or said residue S59 is substituted by an amino acid selected from the group consisting of: I, P, E, L, K, F, R and Y.

5. The mammalian cell or population of cells for use according to claim 1, wherein said cell expressing said first isoform has been selected from a subject comprising native genomic DNA with at least one natural polymorphism allele in nucleic acid encoding said first isoform.

6. The mammalian cell or population of cells for use according to claim 1, wherein said first isoform is obtained by ex vivo modifying the nucleic acid sequence encoding said surface protein by gene editing, preferably by introducing into a cell a gene editing enzyme capable of inducing site-specific mutations(s) within a target sequence encoding surface protein region involved in the binding of agent comprising at least a first antigen-binding region.

7. The mammalian cell or population of cells, according to claim 1, wherein said medical treatment comprises:
administering a therapeutically efficient amount of said cell or population of cells expressing said first isoform to said patient in need thereof, in combination with a therapeutically efficient amount of a depleting agent comprising at least a first antigen-binding region that binds specifically to said second isoform to specifically deplete patient cells expressing second isoform.

8. The mammalian cell or population of cells for use according to claim 7 wherein said depleting agent is an antibody, antibody-drug conjugate or an immune cell.

9. The mammalian cell or population of cells for use according to claim 8 wherein said surface protein is CD123 and wherein said first antigen-binding region of said depleting agent binds specifically to an epitope including the amino acids T48, D49, E51, A56, D57, Y58, 559, M60, P61, A62, V63, N64, T82, R84, V85, A86, N87, P89, F90, S91 of SEQ ID NO: 1.

10. The mammalian cell or population of cells according to claim 1, wherein said medical treatment comprises:
administering a therapeutically efficient amount of said cell or population of cells expressing said first isoform to said patient in need thereof in combination with a therapeutically efficient amount of a depleting agent comprising at least a second antigen-binding region that binds specifically to said first isoform to specifically deplete transferred cells expressing first isoform.

11. The mammalian cell or population of cells for use according to claim 10 wherein said cell or population of cells expressing said first isoform is an immune cell.

12. The mammalian cell or population of cells for use according to claim 11 wherein said CAR comprises an antigen-binding region which binds specifically to an epitope of CD123 located within the third extracellular loop, or within the polypeptide including the amino acids T48, D49, E51, A56, D57, Y58, S59, M60, P61, A62, V63, N64, T82, R84, V85, A86, N87, P89, F90, S91 of SEQ ID NO: 1.

13. A pharmaceutical composition comprising a mammalian cell as defined in claim 1 and a pharmaceutically acceptable carrier.

14. A depleting agent for use in preventing or reducing the risk of severe side effects in a patient having received a cell expressing a first isoform of a surface protein, wherein said patient's native cells express a second isoform of surface protein, and wherein said depleting agent comprises at least a second antigen-binding region which binds specifically to said first isoform and does not bind to said second isoform.

15. A depleting agent for use in selectively depleting the host cells in a patient in need thereof wherein said patient's native cells express a second isoform of a surface protein and wherein said depleting agent comprises at least a first antigen-binding region which binds specifically to said second isoform.
